SEQUENCE LISTING (24 sequences)

SEQ ID NO: 1 - DNA, 22 bp, Artificial sequence
  primer
atcaacgaccccttcattgacc

SEQ ID NO: 2 - DNA, 26 bp, Artificial sequence
  primer
ccagtagactccacgacatactcagc

SEQ ID NO: 3 - DNA, 20 bp, Artificial sequence
  primer
tcaacccccagctagttgtc

SEQ ID NO: 4 - DNA, 20 bp, Artificial sequence
  primer
tgttcttcgttgctgtgagg

SEQ ID NO: 5 - DNA, 20 bp, Artificial sequence
  primer
tgggaaatcgtggaaatgag

SEQ ID NO: 6 - DNA, 21 bp, Artificial sequence
  primer
ctctgaaggactctggctttg

SEQ ID NO: 7 - DNA, 21 bp, Artificial sequence
  primer
atgcaggtccctgtcatgctt

SEQ ID NO: 8 - DNA, 21 bp, Artificial sequence
  primer
cattgggatcatcttgctggt

SEQ ID NO: 9 - DNA, 20 bp, Artificial sequence
  primer
caggctgctgtaacgatgaa

SEQ ID NO: 10 - DNA, 20 bp, Artificial sequence
  primer
gcattcacatctgctgtgct

SEQ ID NO: 11 - DNA, 21 bp, Artificial sequence
  primer
ttggagcgagttgtggattgt

SEQ ID NO: 12 - DNA, 20 bp, Artificial sequence
  primer
gtaggtgagggcttggctga

SEQ ID NO: 13 - DNA, 20 bp, Artificial sequence
  Primer
tgactggaagagcggagagt

SEQ ID NO: 14 - DNA, 20 bp, Artificial sequence
  primer
agacggctgagtagggaaca

SEQ ID NO: 15 - DNA, 21 bp, Artificial sequence
  primer
caccccagcgaagaactcata

SEQ ID NO: 16 - DNA, 21 bp, Artificial sequence
  primer
gccaccattgatagtctctcc

SEQ ID NO: 17 - DNA, 23 bp, Artificial sequence
  primer
ttggaaaccttctccatgtcaga

SEQ ID NO: 18 - DNA, 21 bp, Artificial sequence
  primer
tccccagtgggtgttatagga

SEQ ID NO: 19 - DNA, 21 bp, Artificial sequence
  primer
gtcccagacatcagggagtaa

SEQ ID NO: 20 - DNA, 21 bp, Artificial sequence
  primer
tcggatacttcagcgtcagga

SEQ ID NO: 21 - DNA, 25 bp, Artificial sequence
  primer
ggataccaactattgcttcagctcc

SEQ ID NO: 22 - DNA, 25 bp, Artificial sequence
  primer
aggctccaaatataggggcagggtc

SEQ ID NO: 23 - DNA, 440 bp, Artificial sequence
  guide RNA targeting sequence
acaaagaacacaactgtttcccttactattaaaaggcttacttaatatctttgttttgaaacaataatgagtaaataaatatggcttttcaaattttcatttcagttgcttttgccaagagtcagctatttgacgttggtaactgacaaagtgaaaaagcactttcagaaggttatgagacaagaagatgttagtgagatatggtttgaatatgaaggcacacccctgaaatggtgagtttttcttctttgttaagcactaagtatatgctagctgtaagaaggtatagtgtcctaaaaccattgtgaaaagttctacaaagaaagaaggccaagcatggtacctgtaatcccagcattcaagaggctgaagcagaaaattgccagtttgtggctgccctggtcaatatatcatatcaaataccaggaaagctagcacca

SEQ ID NO: 24 - DNA, 20 bp, Artificial sequence
  single guide RNA targeting sequence
atgaaggcacacccctgaaa

(19) United States
(12) Patent Application Publication
(10) Pub. No.: US 2020/0165608 A1
(43) Pub. Date: May 28, 2020

(54) INHIBITION OF VASCULAR ENDOTHELIAL CELL-MEDIATED PHAGOCYTIC PROCESSES FOR TREATMENT OF DEMYELINATING CONDITIONS

(71) Applicant: FLORIDA STATE UNIVERSITY RESEARCH FOUNDATION, INC., TALLAHASSEE, FL (US)

(72) Inventor: YI REN, TALLAHASSEE, FL (US)

(21) Appl. No.: 16/579,195

(22) Filed: Sep. 23, 2019

Related U.S. Application Data
(60) Provisional application No. 62/770,968, filed on Nov. 23, 2018.

(51) Int. Cl. C12N 15/113; A61P 25/28; A61K 9/00; A61K 38/47

(52) U.S. Cl. CPC C12N 15/113; A61P 25/28; A61K 9/0085; A61K 38/47; C12N 2320/32; C12N 2310/11; C12N 2310/14; C12N 2310/141; C12Y 302/01096

(57) ABSTRACT

The present invention concerns a method for treating a demyelinating condition in a subject, by administering an agent to the subject that inhibits vascular endothelial cell phagocytosis. The method of the invention is useful in treating, for example, a demyelinating condition associated with an injury, such as a spinal cord injury or traumatic brain injury, as well as other demyelinating conditions, such as multiple sclerosis.

### CROSS-REFERENCE TO RELATED APPLICATION

The present application claims the benefit of U.S. Provisional Application Ser. No. 62/770,968, filed Nov. 23, 2018, which is hereby incorporated by reference herein in its entirety, including any figures, tables, nucleic acid sequences, amino acid sequences, or drawings.

### GOVERNMENT SUPPORT

This invention was made with government support under Grant No. DMS-0714589 and Grant No. DMS-1661727 awarded by the National Science Foundation. The government has certain rights in the invention.

### SEQUENCE LISTING

The Sequence Listing for this application is labeled “2OT1984.TXT” which was created on Sep. 20, 2019 and is 8 KB. The entire contents of the sequence listing is incorporated herein by reference in its entirety.

### BACKGROUND OF THE INVENTION

Fibrosis involves the overgrowth, hardening, and/or scarring of various tissues and is attributed to excess deposition of extracellular matrix (ECM) components, including collagen. Fibrosis can be the end result of chronic inflammatory reactions induced by a variety of stimuli including persistent infections, autoimmune reactions, allergic responses, chemical insults, radiation, and tissue injury. The repair of wounds, for example, is a highly complex biological process. After an injury, multiple biological pathways are activated and normally synchronized to respond. The wound repair process commonly leads to production of fibrotic tissue known as a scar. Although tissue fibrosis, or scar formation, is a common response to damage in most organs of the body, and may be required for regaining tissue integrity, it is undesirable in many contexts. For example, fibrosis resulting from nervous tissue is generally viewed as deleterious, as the fibrotic scar is inhibitory to regeneration and recovery of function.

A contusive spinal cord injury (SCI) induces acute mechanical compression of myelin sheath and causes prominent demyelination, a characteristic that is also well documented in multiple sclerosis (MS) and other demyelinating diseases in the central nervous system. The myelin loss, neuronal damage, and spinal microvasculature disruption following SCI trigger a cascade of secondary pathological processes including ischemic injury, inflammation, glial and fibrotic scar formation that prevent tissue regeneration¹. Functional recovery of SCI is ineffective largely due to the failed or incomplete axon and myelin regeneration following SCI^{2, 3}. Myelin debris, which is generated from the breakdown of myelin sheaths immediately after SCI, persists in the injury site and contributes to regeneration failure because myelin debris contains molecules that potently inhibit axon regeneration^{4, 5} and remyelination^{6, 7}. Moreover, myelin debris acts as inflammatory stimuli that exacerbate secondary injury by activating astrocytes, microglia, and macrophages, which are actively involved in inflammatory responses during SCI progression 8-10. Therefore, clearance of myelin debris from the injury site is critical for axon regeneration, remyelination and resolution of inflammation.

Myelin debris is generated within minutes following mechanical trauma and is thought to be cleared mainly by “professional” phagocytes such as bone marrow-derived macrophages (BMDMΦ) and resident microglia¹⁰⁻¹² However, BMDMΦ are not significantly recruited to the injury site until one week after SCI¹⁰ and microglia are generally absent from the lesion epicenter^{10, 13} These observations led to the hypothesis that an alternative phagocytic process performed by “amateur” phagocytes present in the injury core may complement macrophages/microglia for myelin debris clearance, at least in the early stages. Indeed, a recent report shows that astrocytes act as amateur phagocytes to participate in myelin debris clearance in multiple sclerosis (MS)¹⁴. However, this cannot be the case for SCI because astrocytes are absent from the epicenter of injured spinal cords.

Microvessels are present in the injury core as early as 3 days post-injury and their density increases up to 540% of that of normal conditions during the chronic phase of SCI^{15, 16}. Following acute injury, the newly formed microvessels arise by angiogenesis, or proliferation of microvascular endothelial cells (ECs). Microvascular ECs, the lining of microvessels, are generally viewed as a physical barrier to the neurovasculature that restricts the entry of blood-borne toxins and pathogens into underlying tissues, thereby protecting tissues from injury and disease. However, it is known that ECs could act as amateur phagocytes to engulf large particles such as bacteria¹⁷, apoptotic cell bodies¹⁸ and latex particles¹⁹.

### BRIEF SUMMARY OF THE INVENTION

It has been determined that microvessels and lining microvascular endovascular cells (ECs) act as amateur phagocytes to engulf myelin debris generated by disorders associated with demyelination. Mechanistically, the inventor determined that immunoglobulin G (IgG) opsonization of myelin debris is required for efficient uptake by microvascular ECs. The engulfed myelin debris is then delivered through the autophagy-lysosome pathway for intracellular degradation. Functionally, engulfment and autophagy-dependent processing of myelin debris by microvascular ECs contribute to three critical processes that are closely associated with demyelinating disorders: robust angiogenesis that results in excessive and abnormal microvessels, chronic inflammation, and endothelial-mediated fibrosis that most likely takes place through endothelial-to-mesenchymal transition (endoMT). Therefore, the inventor proposes that a benefit to patients can be obtained by intervening and interfering with the effects of myelin-ECs by targeting these particular processes (e.g., myelin debris uptake, autophagy and endoMT).

The invention concerns a method for treating a demyelinating condition in a human or animal subject, wherein the method comprises administering an agent or treatment to the subject that inhibits vascular endothelial cell phagocytosis. In some embodiments, the demyelinating condition is associated with a wound or injury, such as a neural injury (an injury to nervous tissue). For example, the neural injury may be a neuropraxia, axonotmesis, or neurotmesis. The neural injury may be an injury nervous tissue of the peripheral nervous system (PNS), central nervous system (CNS), or both. For example, the injury may be a spinal cord injury (SCI).

In some embodiments, the subject has a demyelinating condition at the time the agent is administered, and the agent is administered as therapy for the demyelinating condition. In other embodiments, the subject does not have a demyelinating condition at the time the agent is administered, and the agent is administered as prophylaxis to prevent or delay the onset of the demyelinating condition, including recurrence of a previous demyelinating condition. By treatment of a demyelinating condition in a subject, it is possible to reduce, prevent, or delay the onset of fibrosis, or a fibrotic condition, associated with vascular endothelial cell phagocytosis.

The method of the invention involves administering an agent to the subject that inhibits vascular endothelial cell phagocytosis in the subject. The agent may be administered locally or systemically. In some embodiments, the agent is administered locally at a desired anatomical site, such as a site of demyelination or a site at risk of demyelination. For example, local administration to the desired anatomical may be by direction injection or topical application.

In some embodiments, the agent or treatment inhibits the autophagy-lysosome pathway in vascular endothelial cells of the subject. In some embodiments, the agent that inhibits the autophagy-lysosome pathway is an agent that inhibits ATG5 in vascular endothelial cells.

In some embodiments, the agent that inhibits vascular endothelial cell phagocytosis in the subject is an agent that depletes or inactivates IgG. For example, an agent that inactivates IgG may be administered, such as an enzyme that hydrolyzes IgG (for example, the endoglycosidase EndoS or the protease IdeSt). The agent that depletes IgG may be, for example, a B-cell-attenuating agent, such as bortezomib or rituximab.

### BRIEF DESCRIPTION OF THE DRAWINGS

The patent or application file contains at least one drawing executed in color. Copies of this patent or patent application publication with color drawing(s) will be provided by the Patent and Trademark Office upon request and payment of the necessary fee.

FIGS. 1A, 1A-1, 1B, 1B-1, 1C, 1C-1, 1D, 1E, 1E-1, 1F, and 1F-1. Engulfment of myelin debris by spinal microvessels in SCI and EAE mouse models and in vitro in endothelial cells-induced microvessels-like structures. (FIGS. 1A, 1B, 1C) Internalization of myelin debris (MBP staining, green) by microvessels (CD31 staining, red) in normal spinal cords from uninjured mice (FIG. 1A) and spinal cords from injured mice after 1 week of SCI (FIG. 1B) or 1 week of EAE (FIG. 1C). The x-y, x-z and y-z views (FIGS. 1A-1, 1B-1, 1C-1) show myelin debris was internalized by microvessels in SCI and EAE spinal cords. (FIG. 1D) Quantification of myelin-containing microvessels in normal, SCI and EAE spinal cords. Myelin uninjured region and myelin injured region were classified in SCI and classification details are in materials and methods. Data are shown as means±s.e.m. n=4 in SCI, n=3 in EAE. 1-d SCI, p=0.779 (ns, not significant); 3-d SCI, p=0.0111 (*); 5-d SCI, p=0.0088 (**); 7-d SCI, p=0.0121 (*); 7-d EAE, p=0.0076 (**). p<0.05 (*), p<0.01 (**) by paired Student's t-test. (FIGS. 1E, 1E-1) Detection of neutral lipids (ORO, red) in spinal microvessels from mice after 2 weeks of SCI. (FIG. 1E-1) 3D reconstruction of microvessel (CD31, green) shows accumulation of ORO⁺ lipids inside microvessels. (FIGS. 1F, 1F-1) Distribution of CFSE-labeled myelin debris (green) in primary BMECs-assembled microvessels-like structures (CD31, red) on Matrigel that were incubated with myelin debris for 72 hr. The x-y and y-z views of one region of interest show myelin debris approaching to but not contacting microvessels-like structures (FIG. 1F-1, number 1), or starting to touch (FIG. 1F-1, number 2) or entering (FIG. 1F-1, number 3) microvessels-like structures. Scale bar, 50 μm (FIGS. 1A, 1B, 1C), 20 μm (FIGS. 1A-1, 1B-1, 1C-1), 10 μm (FIG. 1E), 50 μm (FIG. 1F), 5 μm (FIG. 1F-1).

FIGS. 2A-2I. In vitro engulfment of myelin debris by brain microvascular endothelial cells (BMECs). (FIG. 2A) Representative confocal images showing engulfment of CFSE-labeled myelin debris (green) by primary BMECs (CD31, red) after exposure to myelin debris for the indicated time points. Scale bar, 20 μm. (FIG. 2B) Flow cytometry detection of the percentage of myelin-laden primary BMECs with or without CFSE-myelin debris treatment for 72 hr. Data are shown as means±s.e.m. (n=3). p<0.0001 (****) by unpaired Student's t-test. (FIG. 2C) ELISA detection of intracellular MBP in primary BMECs treated with or without myelin debris for 72 hr. Data are shown as means±s.e.m. (n=3). p=0.0144 (*) by unpaired Student's t-test. (FIG. 2D) Representative confocal images showing engulfment of CFSE-myelin debris (green) by bEnd.3 cell line (CD31, red) after myelin debris treatment for the indicated time points. Scale bar, 20 μm. (FIG. 2E) FACS detection of the percentage of myelin-laden BMECs in bEnd.3 cell line at the indicated time points. Data are shown as means±s.e.m. (n=3). Unpaired Student's t-test. 24 hr vs 0 hr, p<0.0001 (****); 48 hr vs 0 hr, p=0.0004 (***); 72 hr vs 0 hr, p<0.0001 (****); 96 hr vs 0 hr, p<0.0001 (****); 96 hr vs 72 hr, p=0.1308 (ns). (FIG. 2F) ELISA detection of intracellular MBP in bEnd.3 cell line at the indicated time points. Data are shown as means±s.e.m. (n=3). Unpaired Student's t-test. 24 hr vs 0 hr, p=0.1939 (ns); 48 hr vs 0 hr, p=0.0173 (*); 72 hr vs 0 hr, p=0.0006 (***); 96 hr vs 0 hr, p=0.0008 (***); 96 hr vs 72 hr, p=0.1884 (ns). (FIG. 2G) Representative confocal images showing CFSE-myelin debris uptake by BMECs pretreated with control IgG, CR3 or Mac-2 neutralizing antibodies. Scale bar, 20 μm. (FIG. 2H) Representative images showing BMECs engulfment of CFSE-myelin debris in serum heated at 56° C. for 20 min (to inactivate C3, left), at 70° C. for 20 min (to inactivate IgG, middle) and engulfment of IgG-opsonized myelin debris in IgG inactivated serum (right). Scale bar, 20 μm. (FIG. 2I) Corresponding quantification was analyzed by ELISA detection of intracellular MBP in BMECs after treatment with myelin debris, IgG-opsonized myelin debris for 72 hr in the medium containing normal serum, C3-inactivated serum, no serum, IgG-inactivated serum or IgG-supplemented serum. Data are shown as means±s.e.m. (n=3). Unpaired Student's t-test. p=0.0005 (***), p=0.0011 (**) were calculated in comparison to normal serum group by unpaired Student's t-test. Among no serum groups, IgG-opsonized myelin vs control, p=0.0001 (^{###}); IgG supplementation vs control, p<0.0001 (^{####}). Among IgG inactivated groups, IgG-opsonized myelin vs control, p=0.0013 (^{##}); IgG supplementation vs control, p=0.0029 (^{##}).

FIGS. 3A-3E. Transcriptome comparison of naïve-BMECs and myelin-BMECs. (FIG. 3A) Heatmap comparing naïve-BMECs and myelin-BMECs for the top 50 most changed genes. Two biological replicates were used for RNA-seq analysis. (FIGS. 3B, 3C) The upregulated and downregulated genes enriched in different groups in myelin-BMECs. Values show log₂-fold changes. The differentially expressed genes are statistically significant. P-value and adjusted p-value are in Table 1. (FIGS. 3D, 3E) Quantitative RT-PCR analysis of gene expression related to pro-fibrotic (FIG. 3D) and inflammatory responses (FIG. 3E) in naïve-BMECs and myelin-BMECs. Data are shown as means±s.e.m. (n=3). Collagen 1α2, p=0.0132 (*); Collagen 1α1, p=0.0327 (*); Collagen 5α2, p=0.0057 (**); MCP-1, p<0.0001 (****); IL-6, p<0.001 (***); IL-4, p=0.0176 (*); iNOS, p=0.0004 (***) by unpaired Student's t-test.

FIGS. 4A, 4A-1, 4B, 4C, 4D, 4D-1, 4E, 4F, 4F-1, 4F-2, 4G, 4H, 4I, 4J, and 4K.

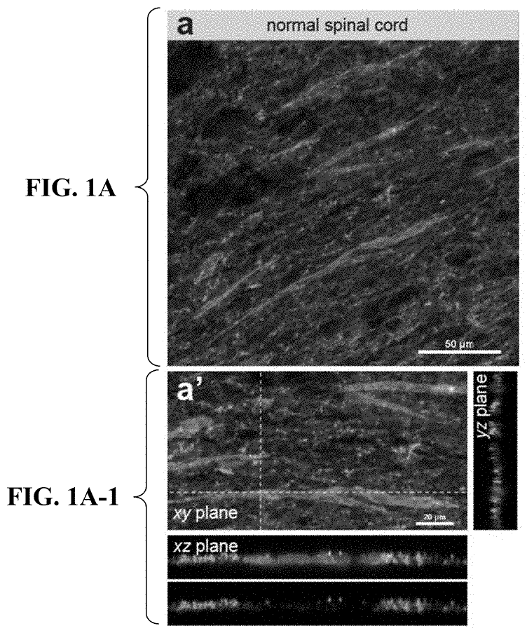

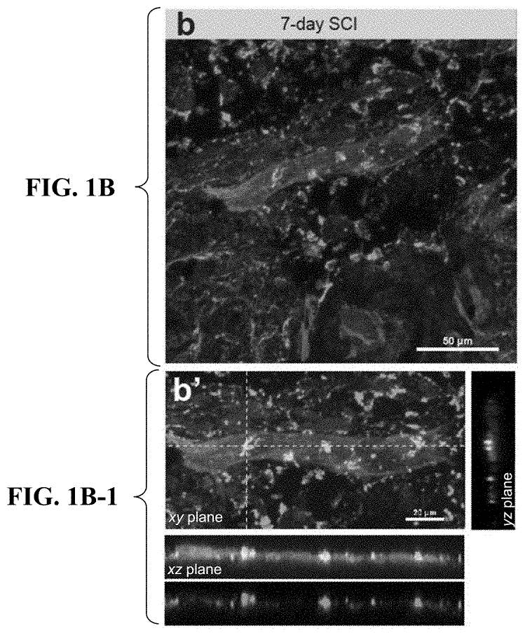

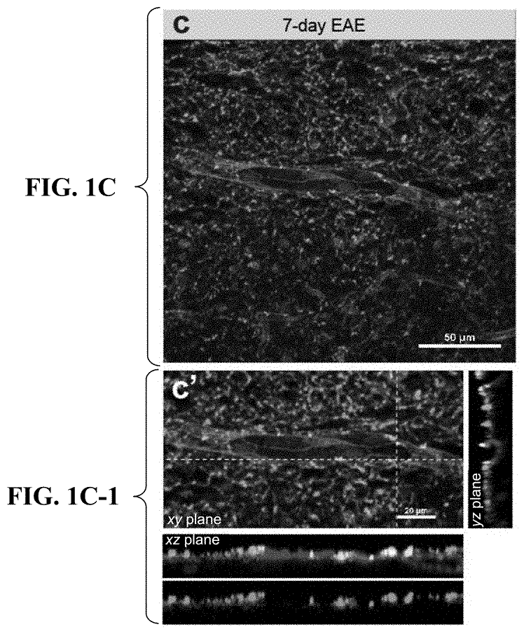

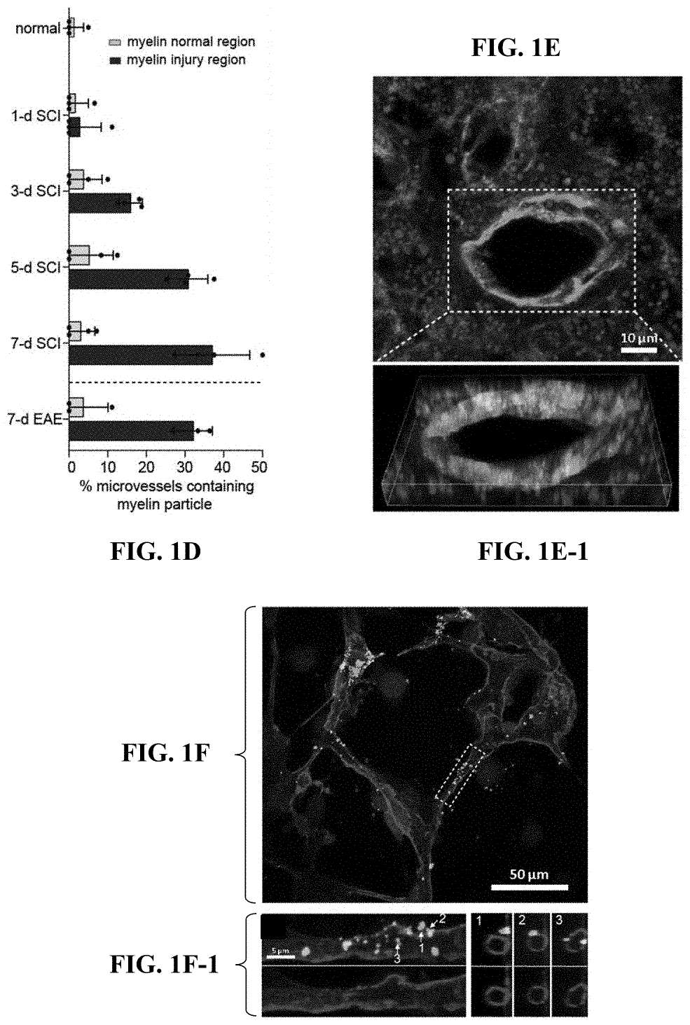

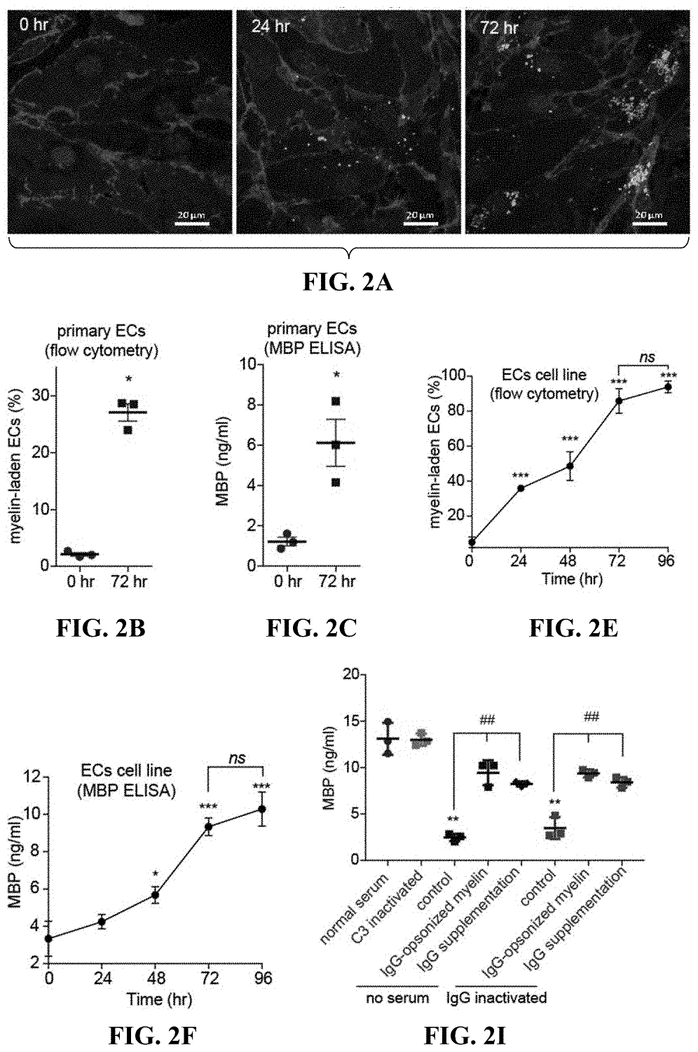

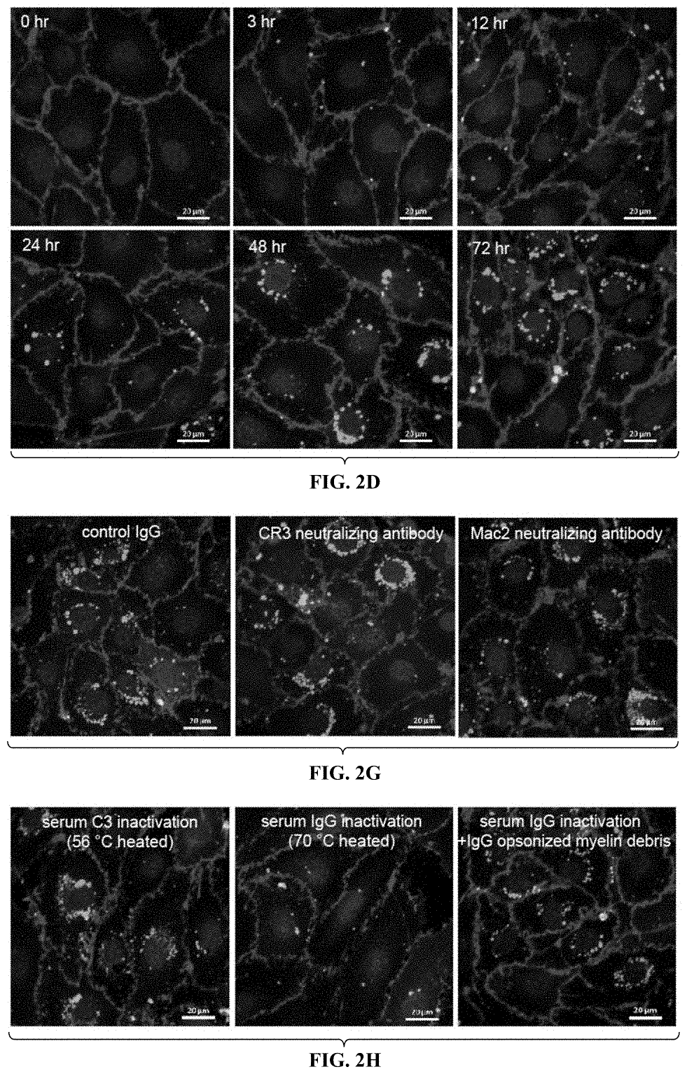

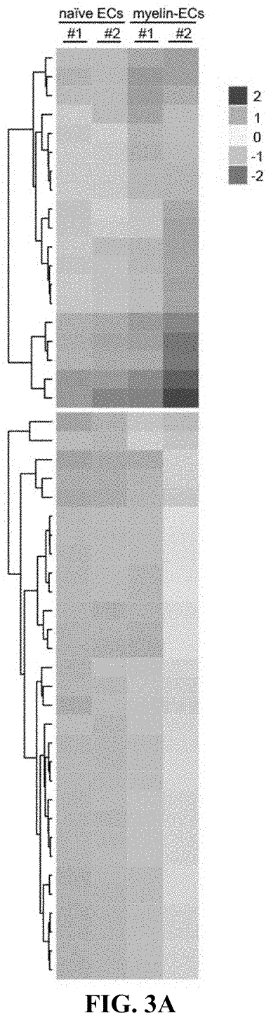

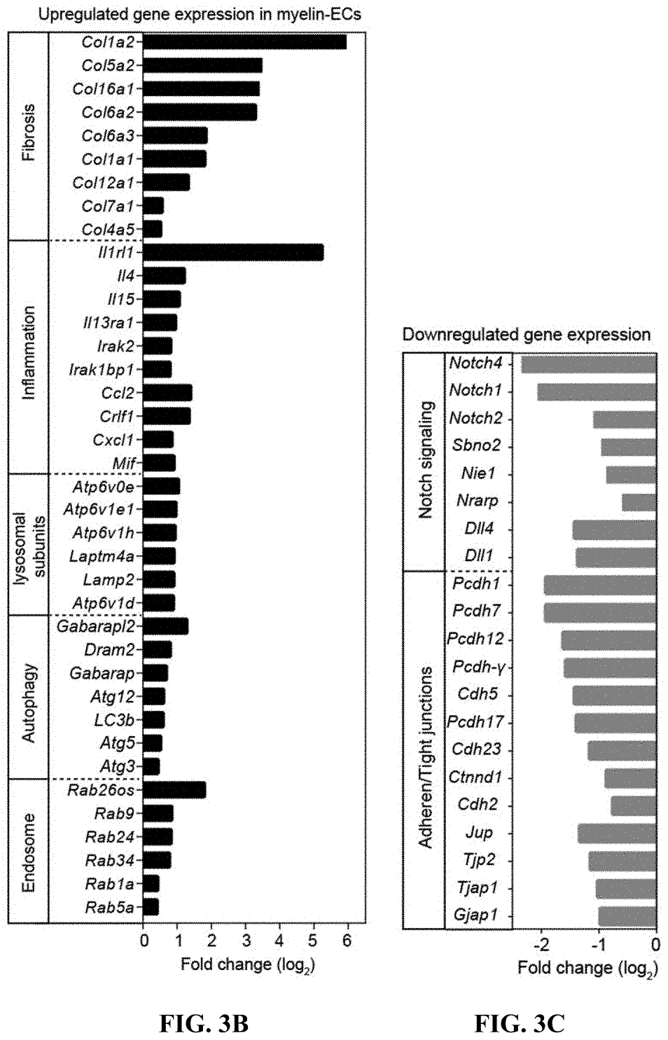

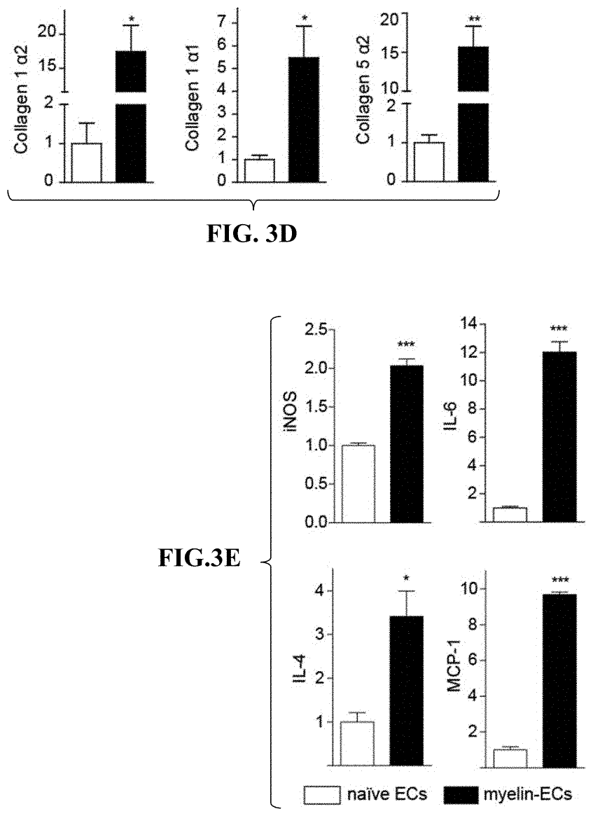

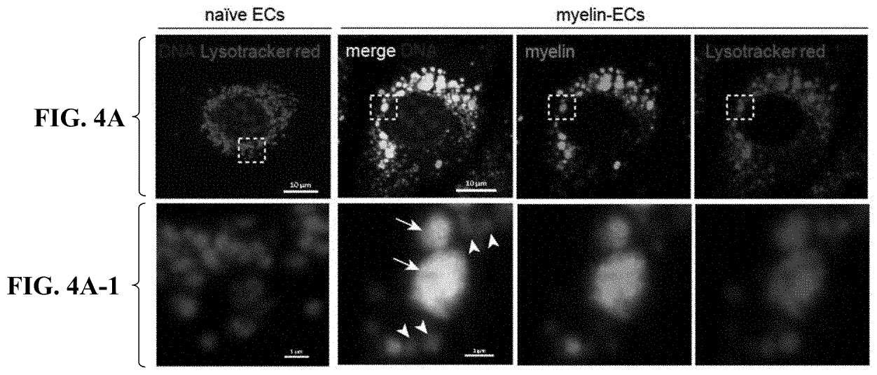

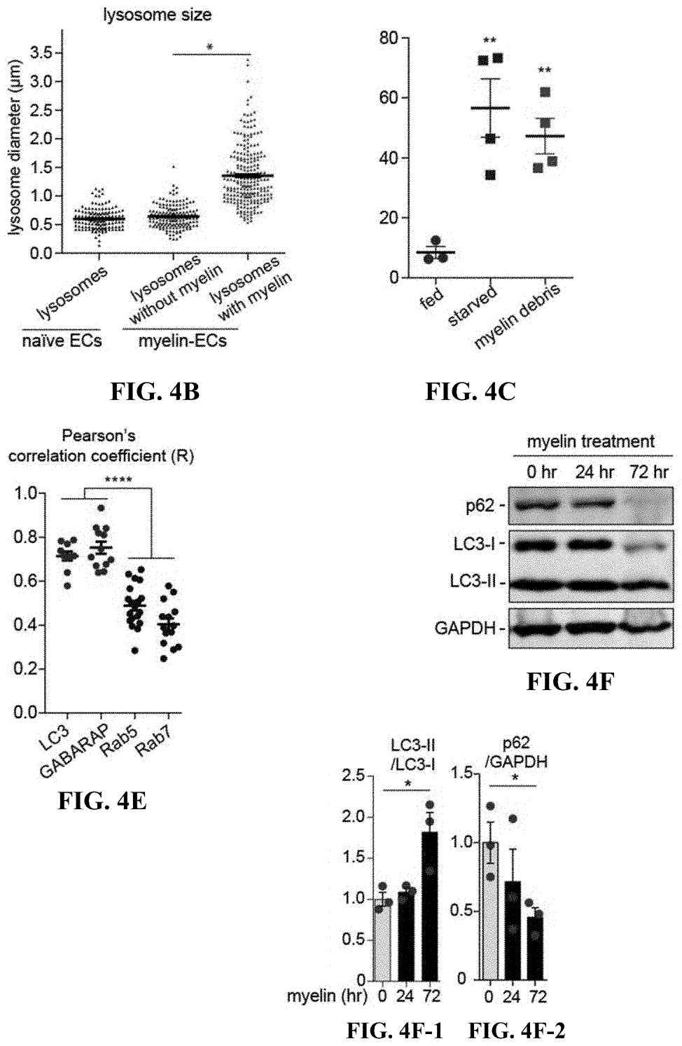

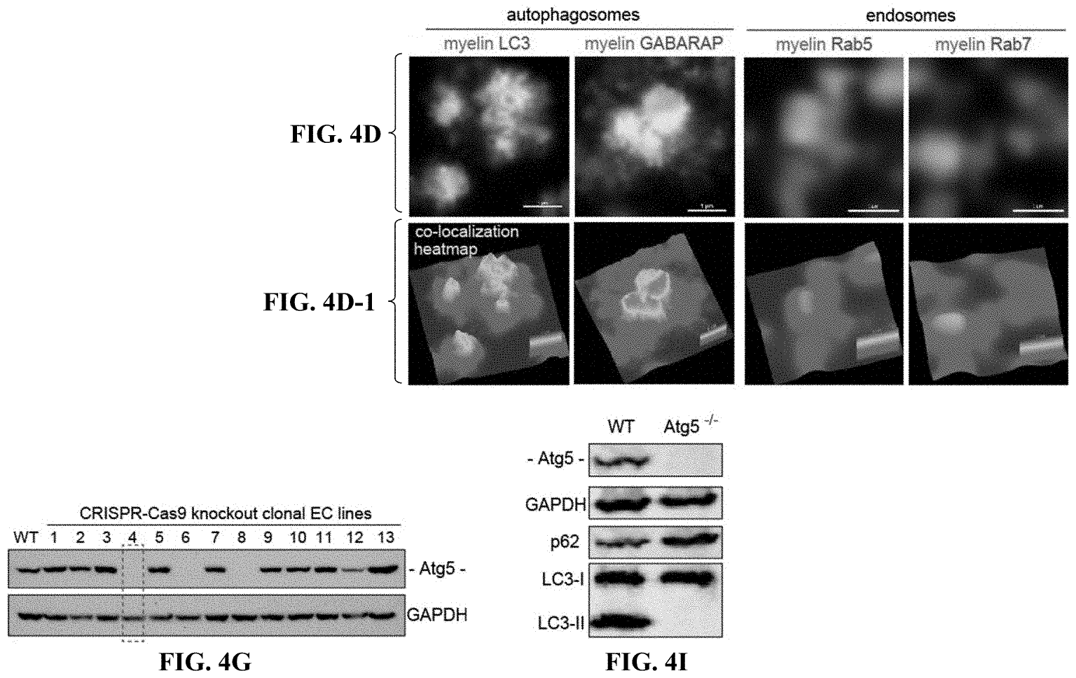

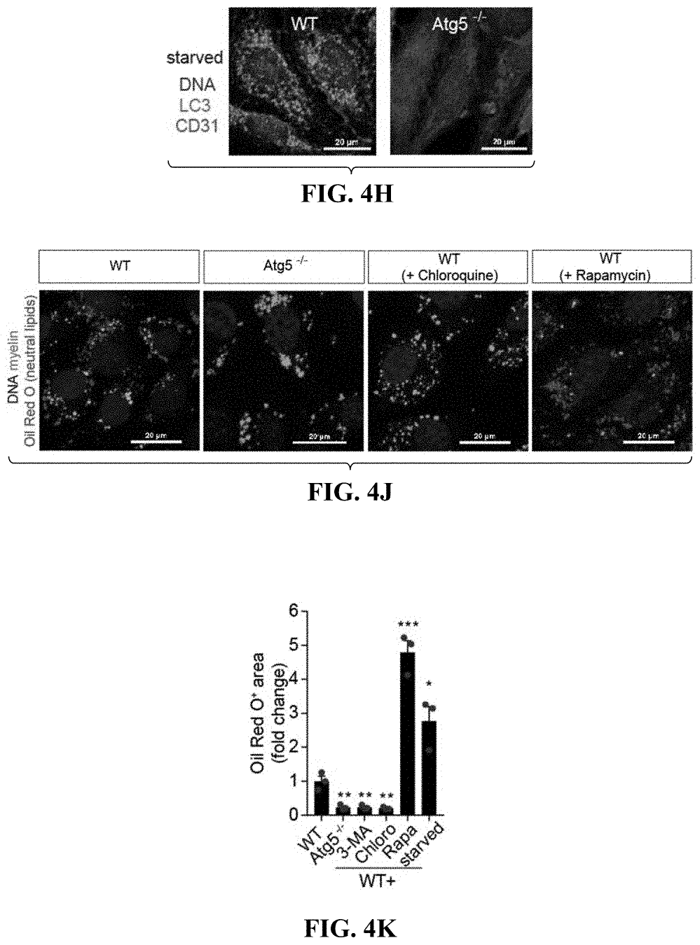

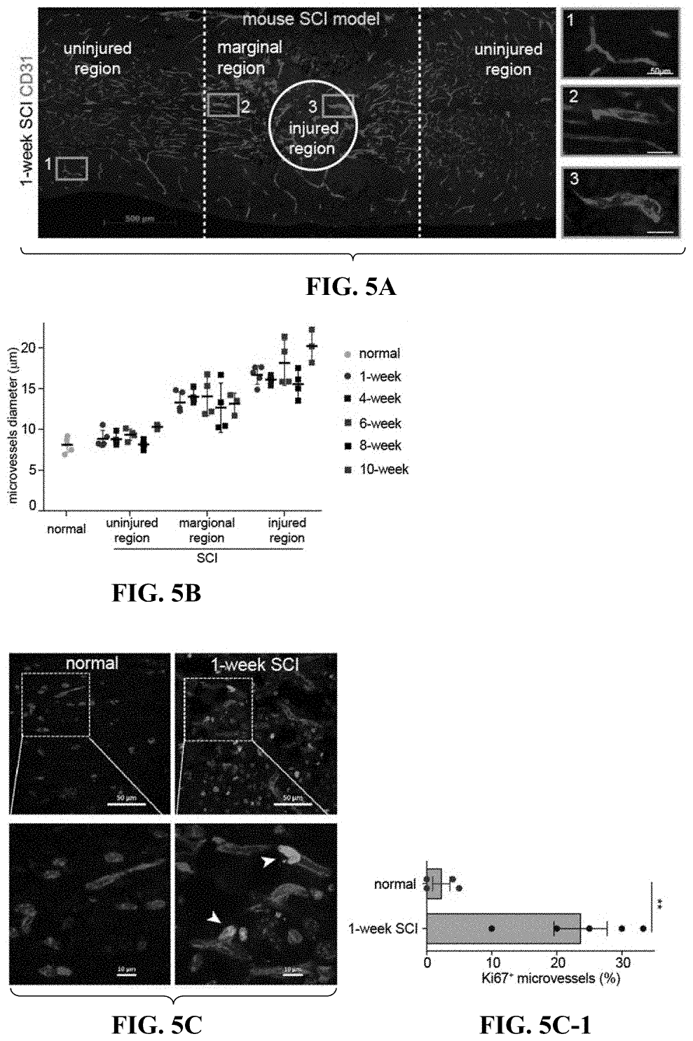

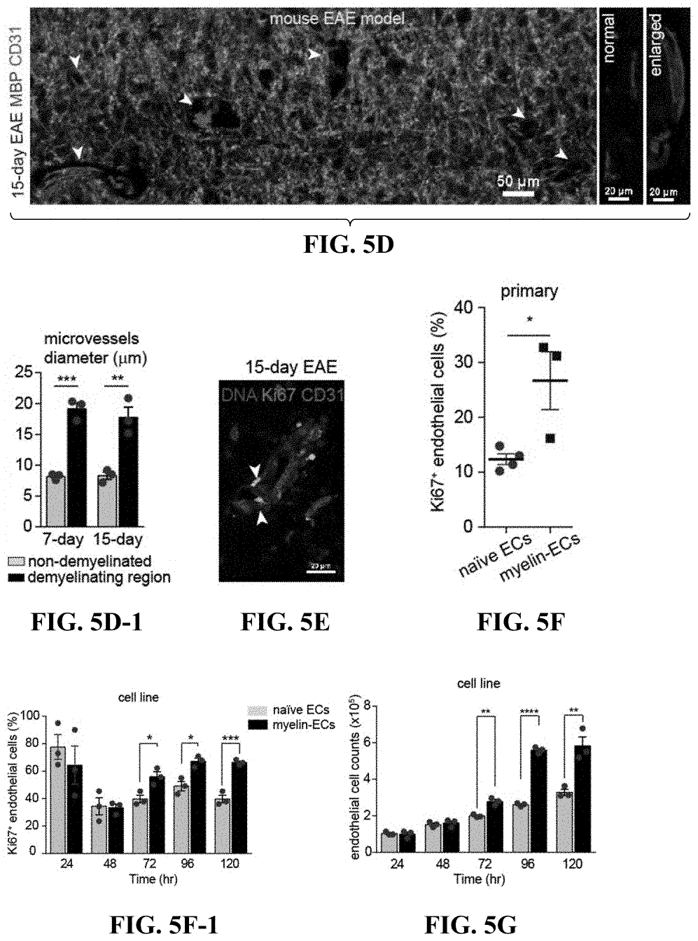

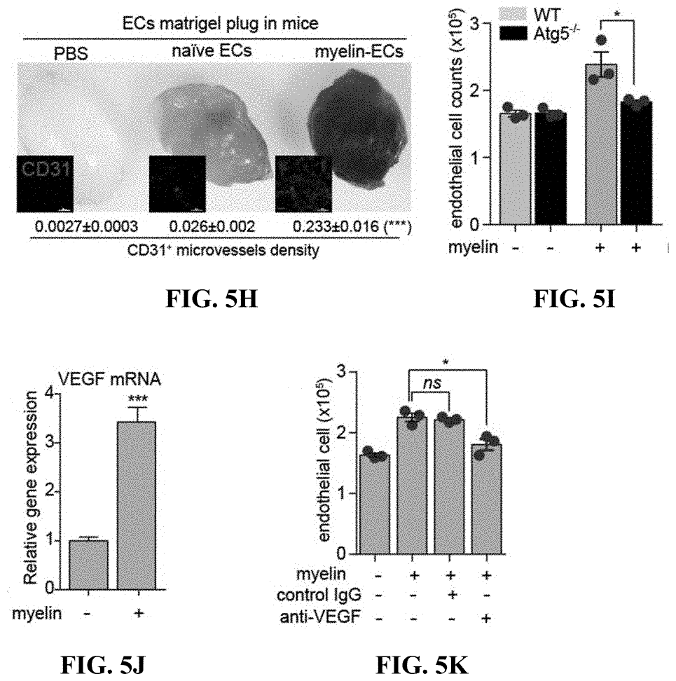

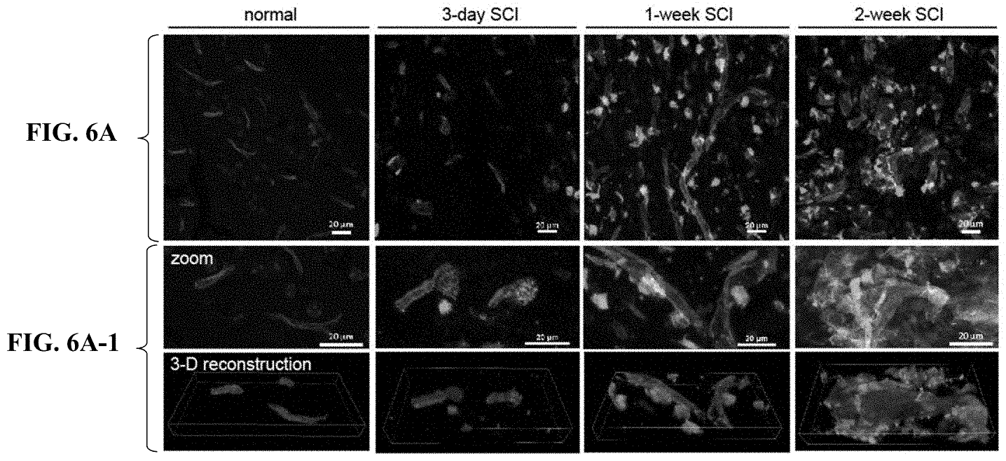

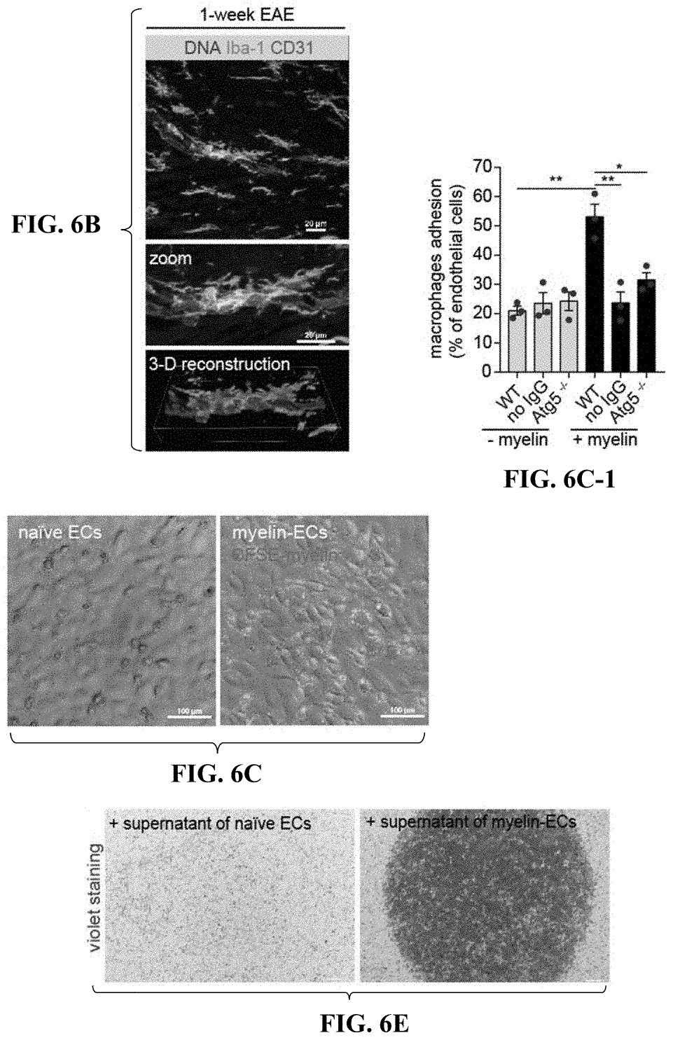

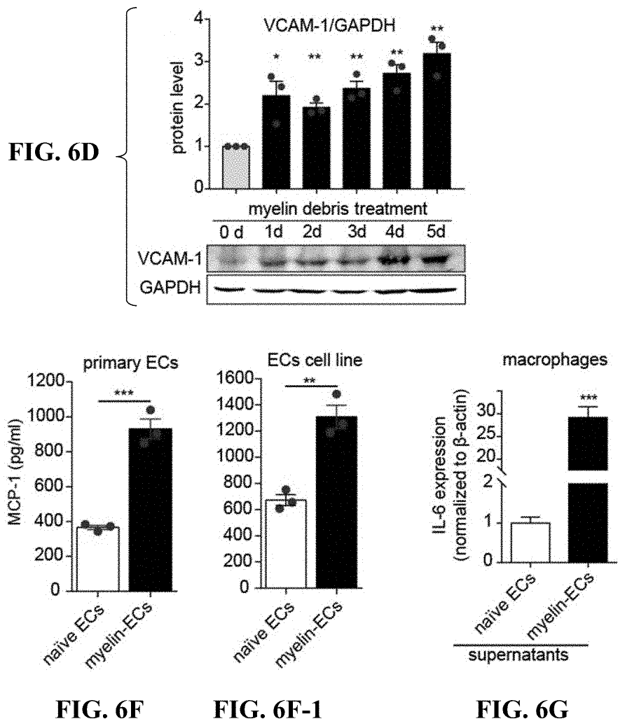

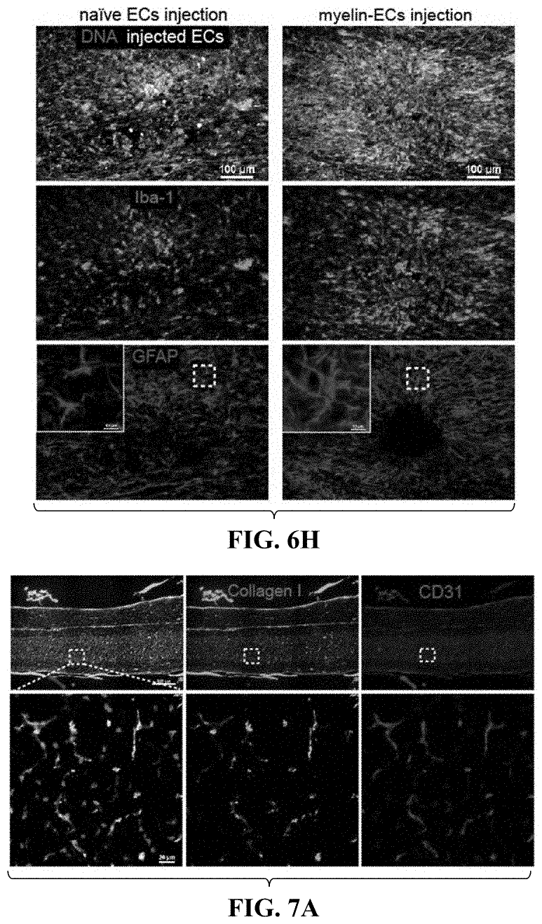

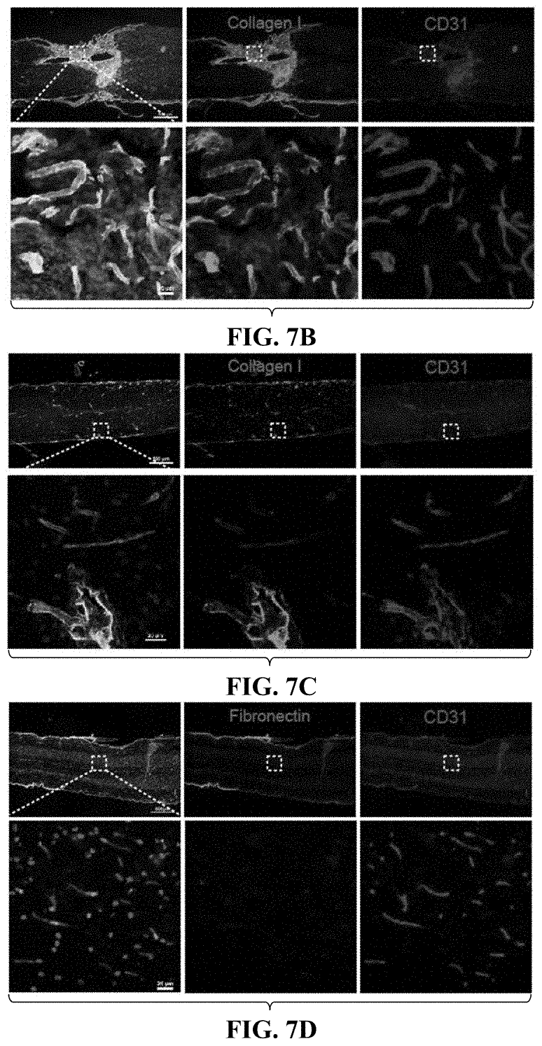

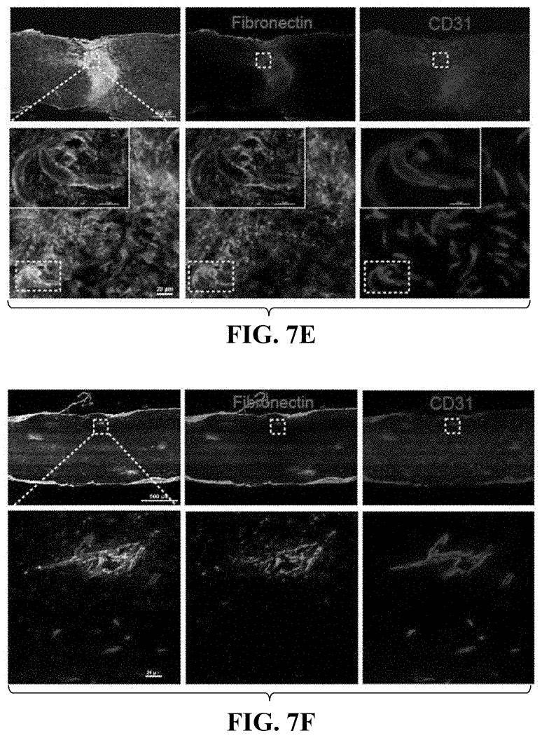

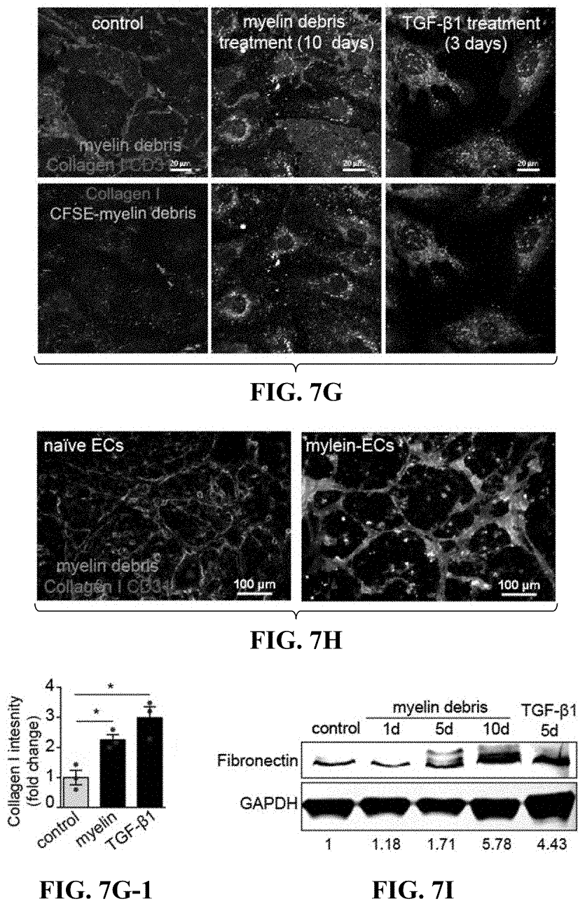

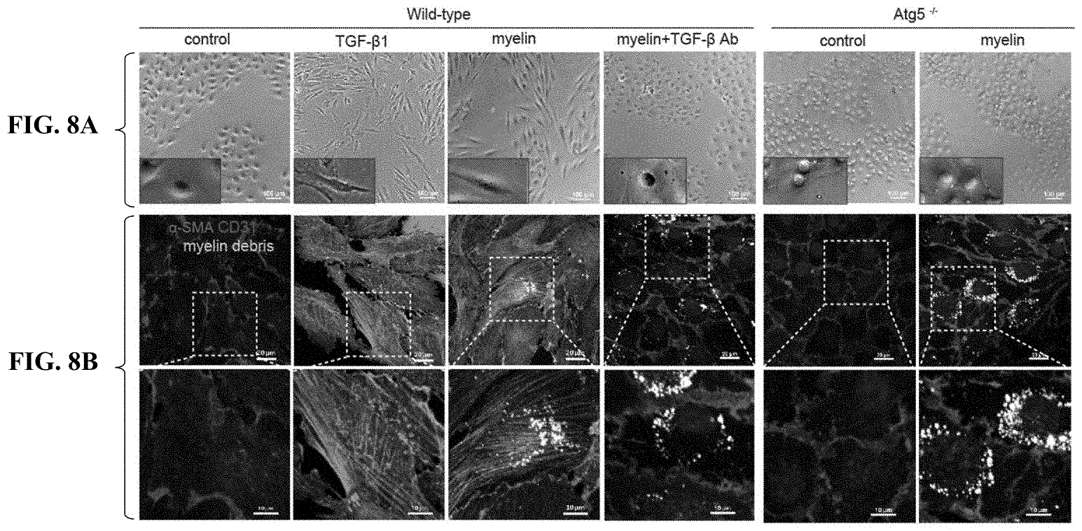

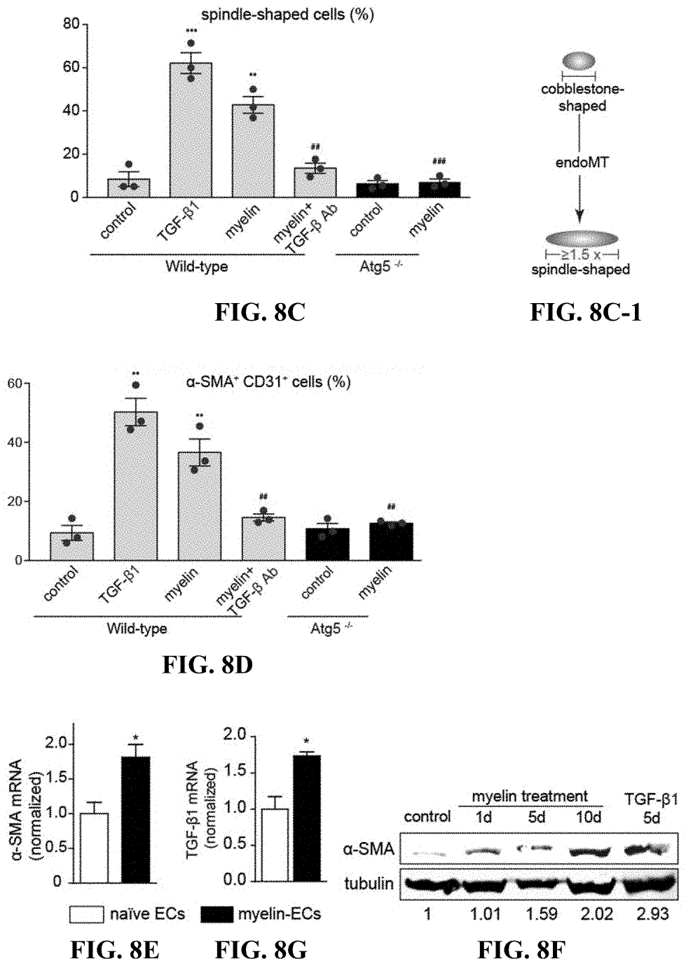

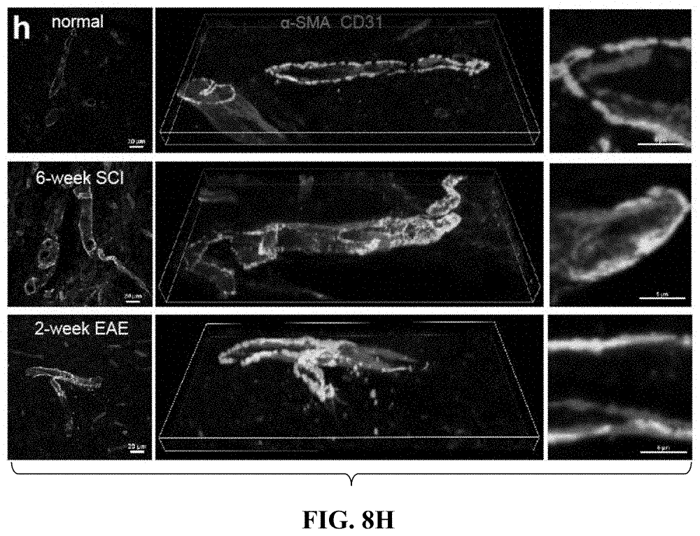

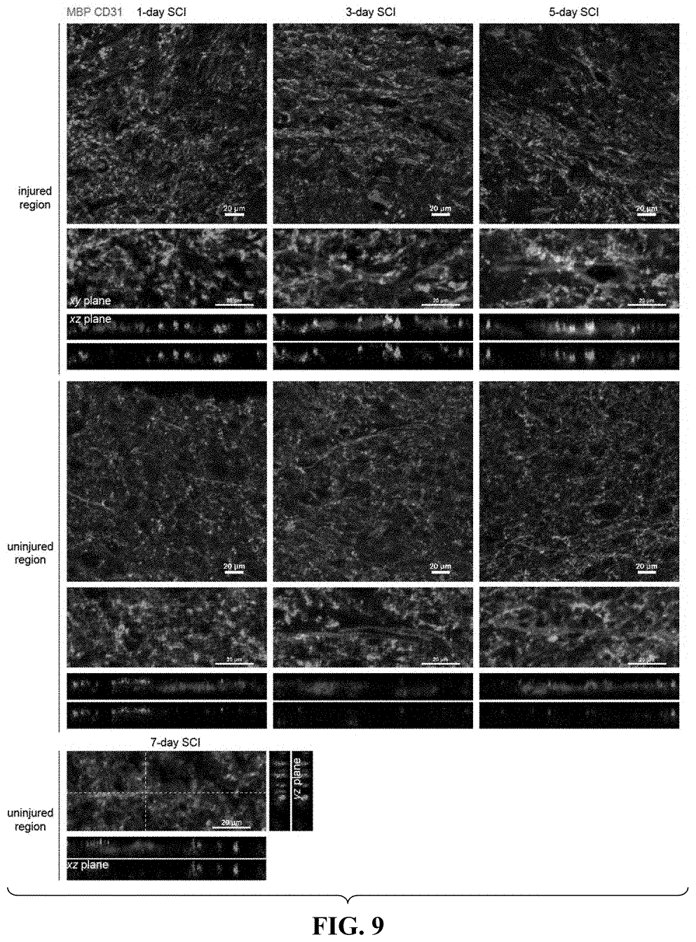

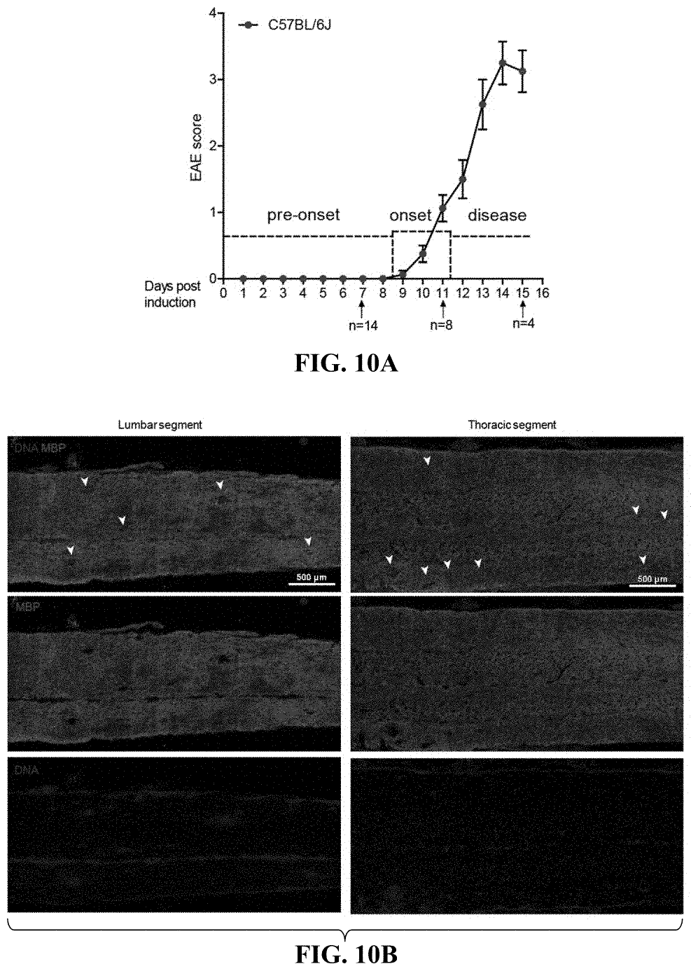

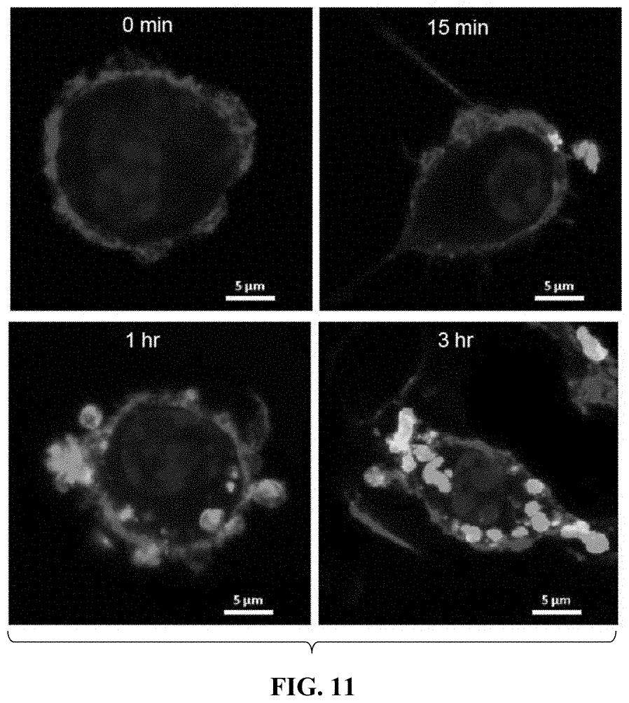

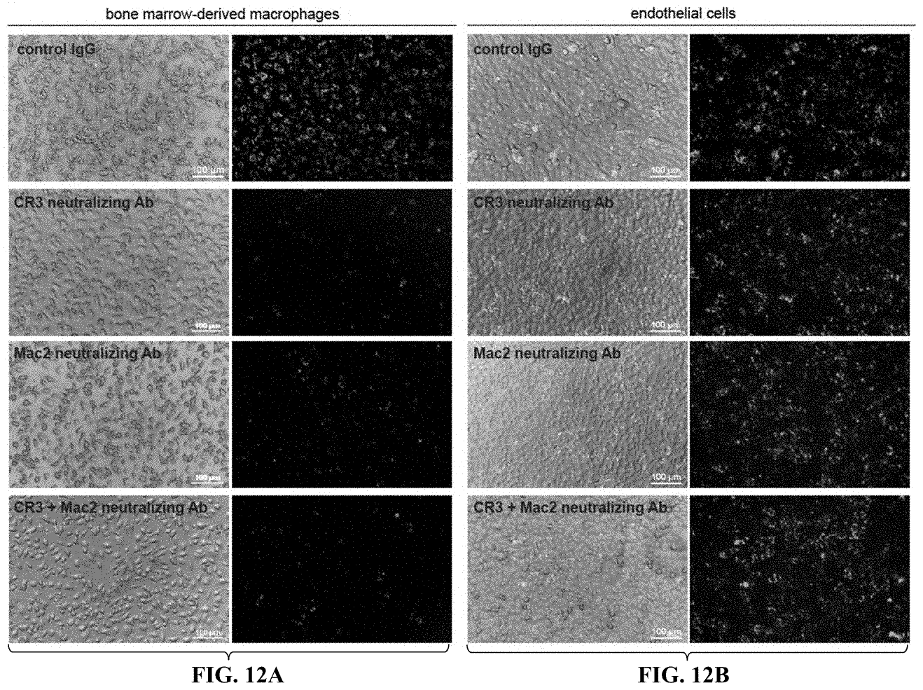

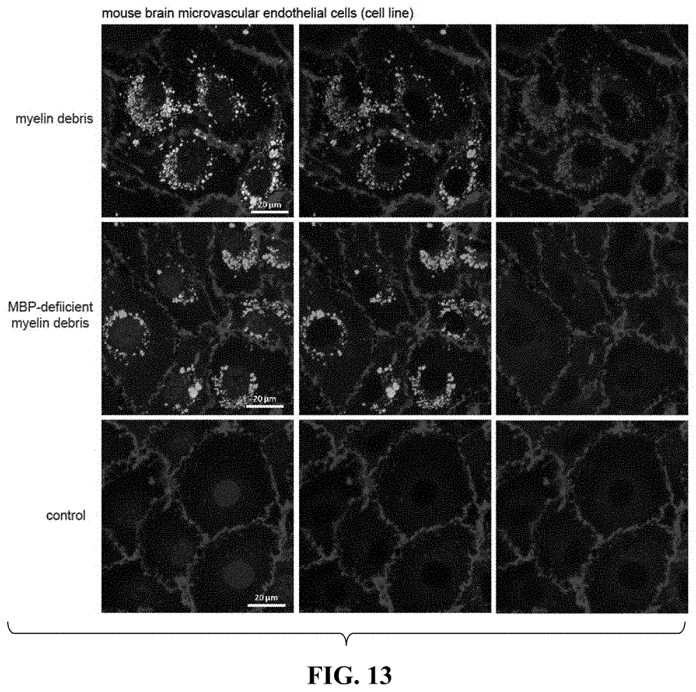

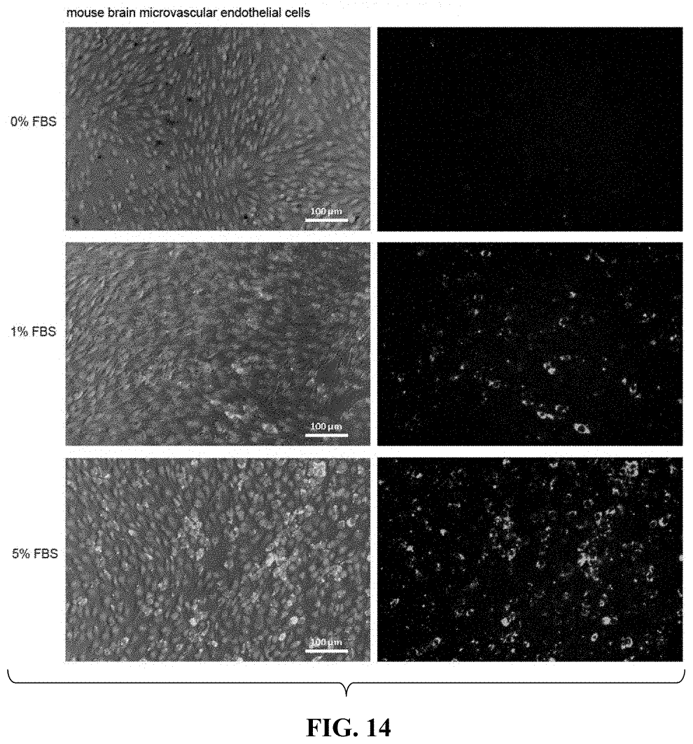

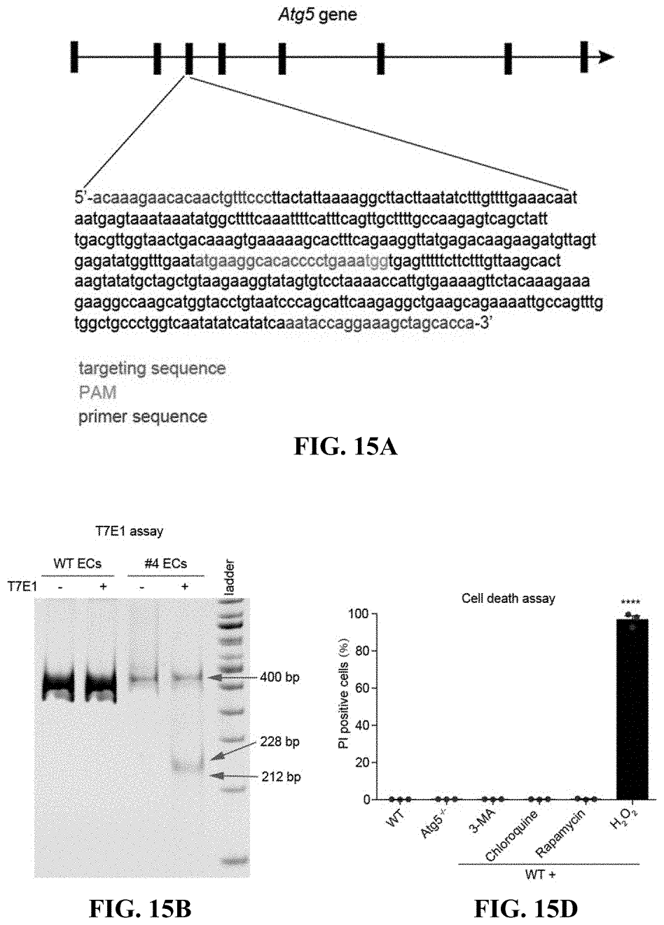

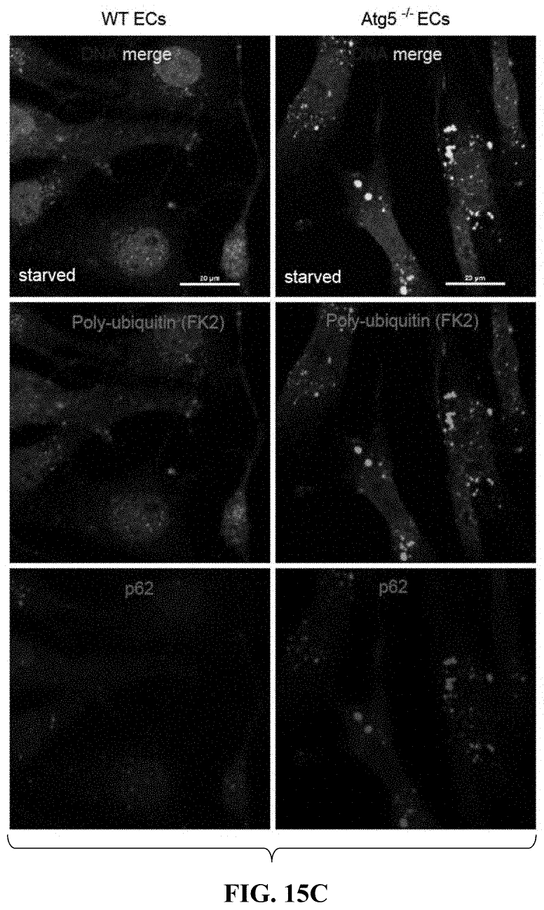

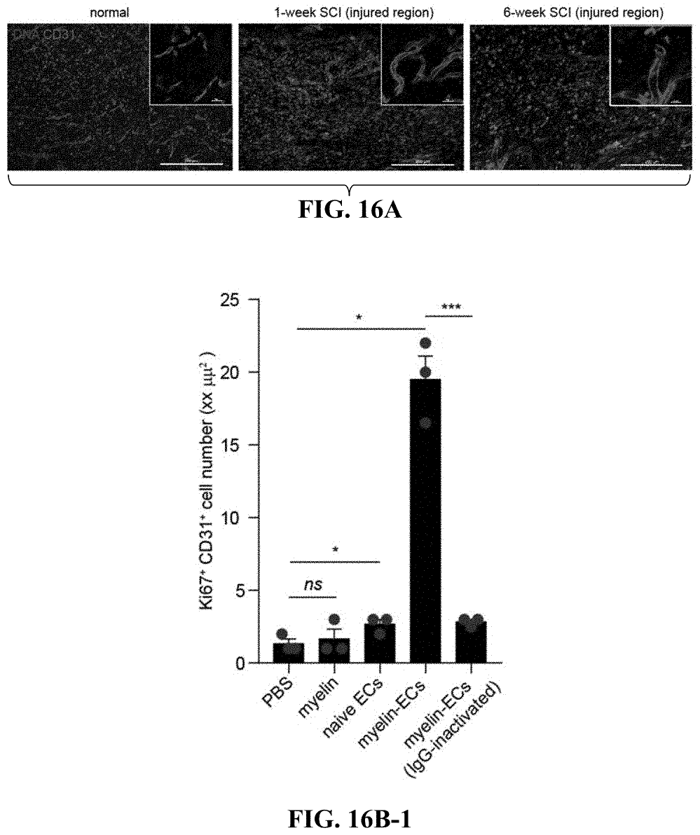

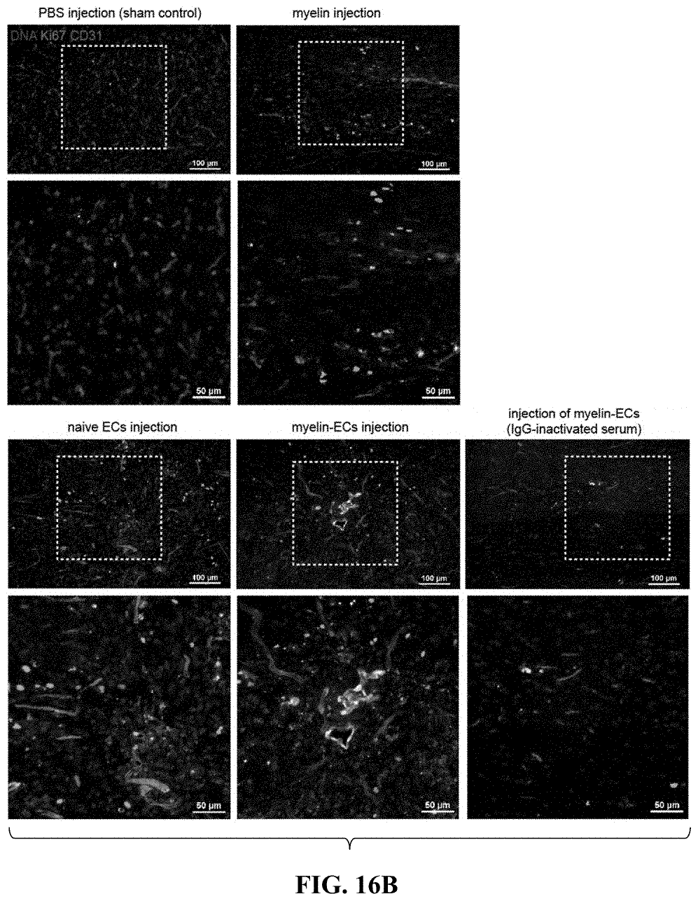

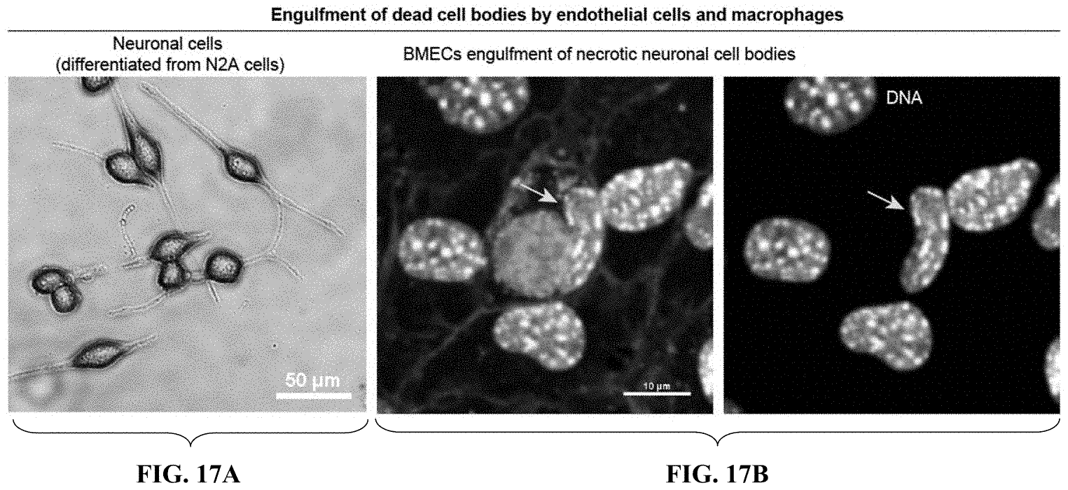

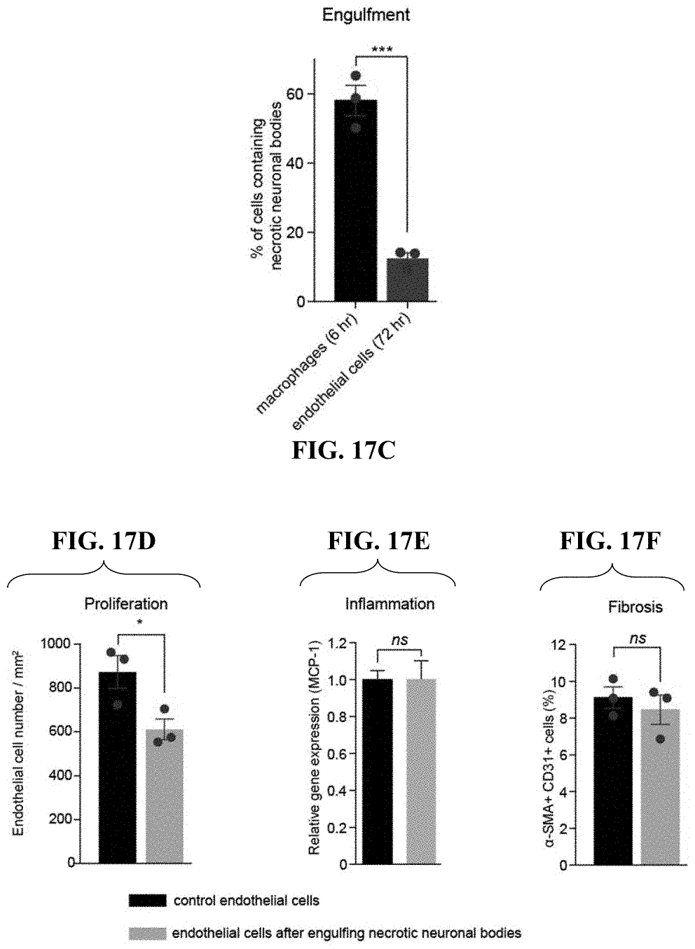

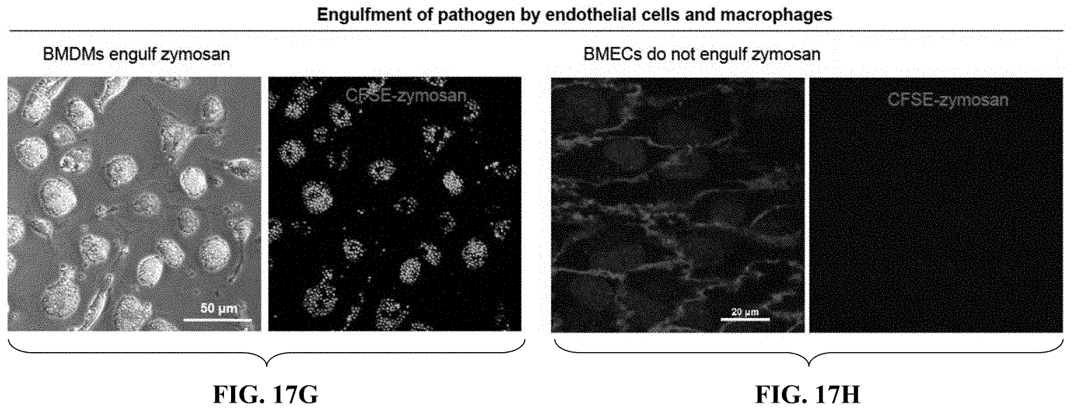

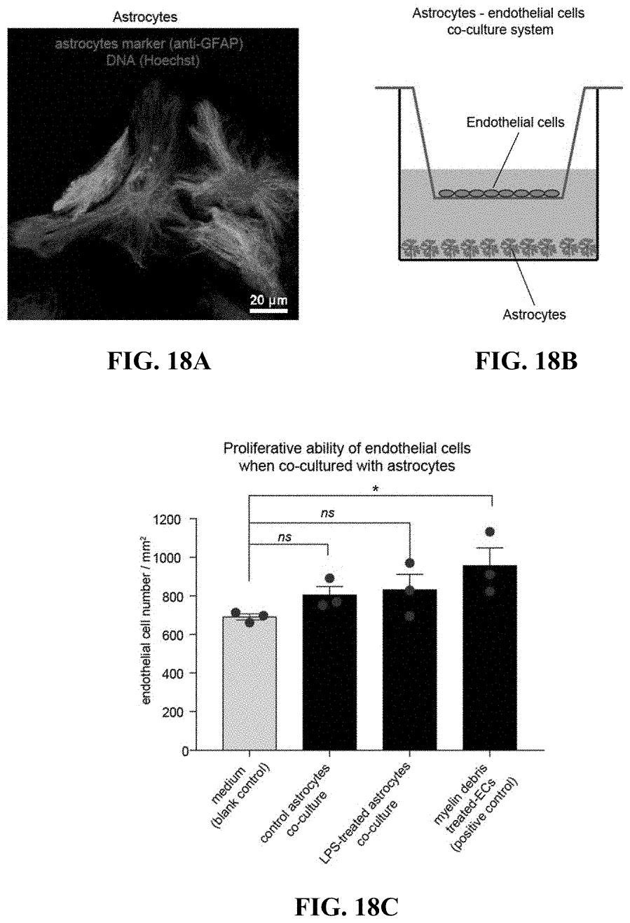

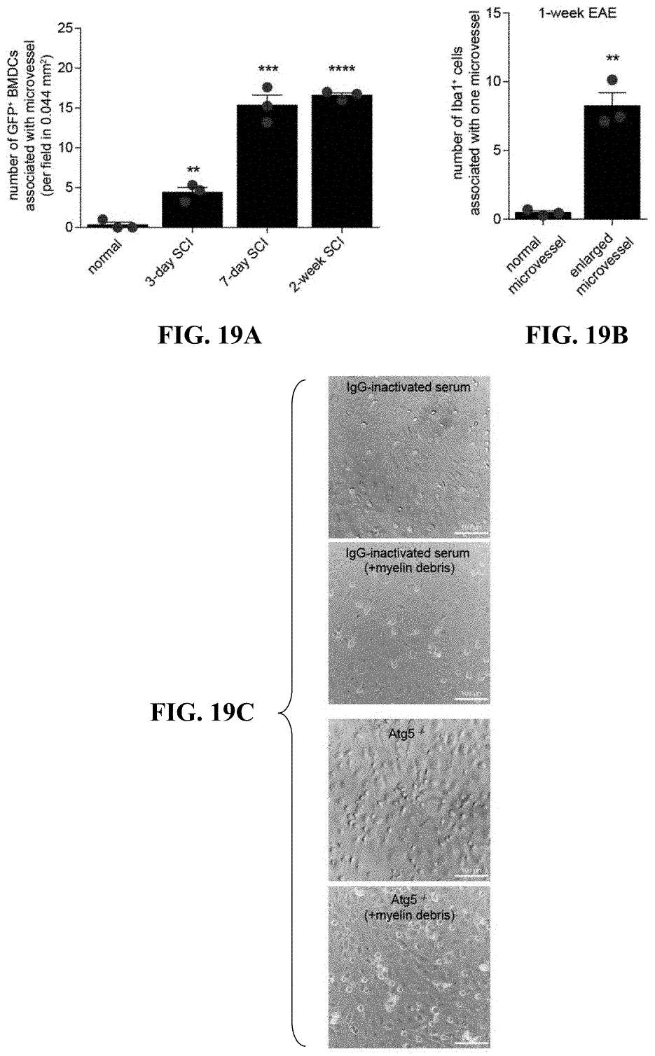

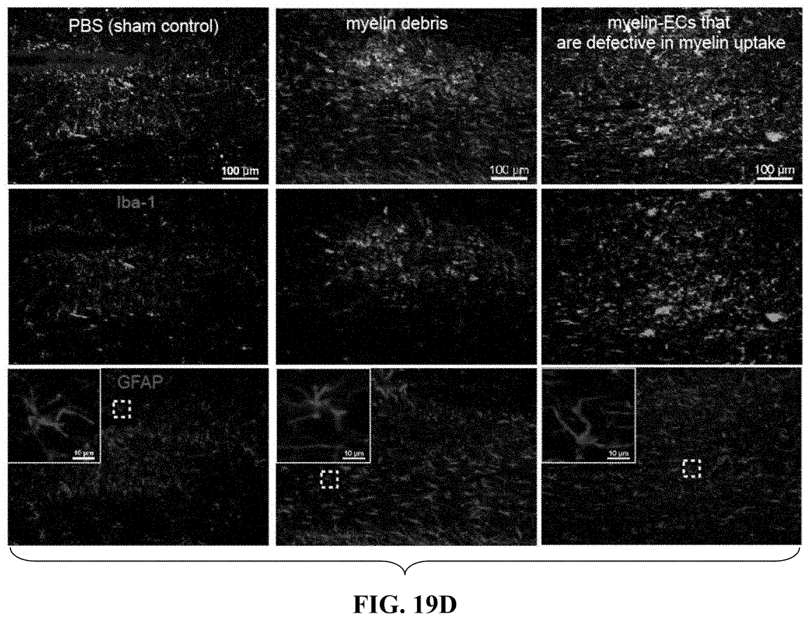

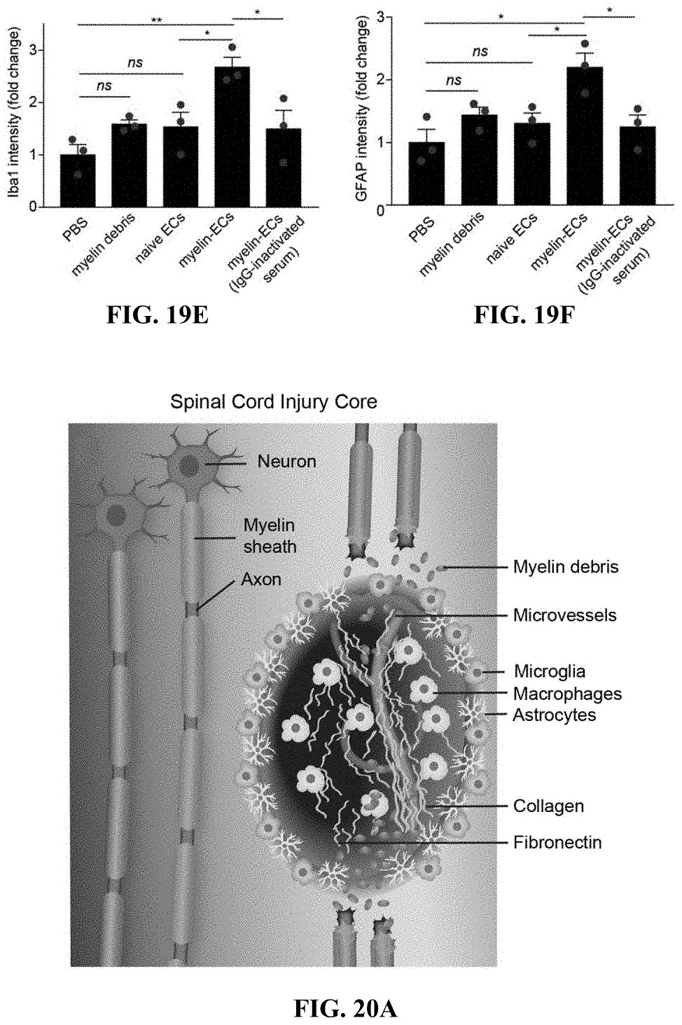

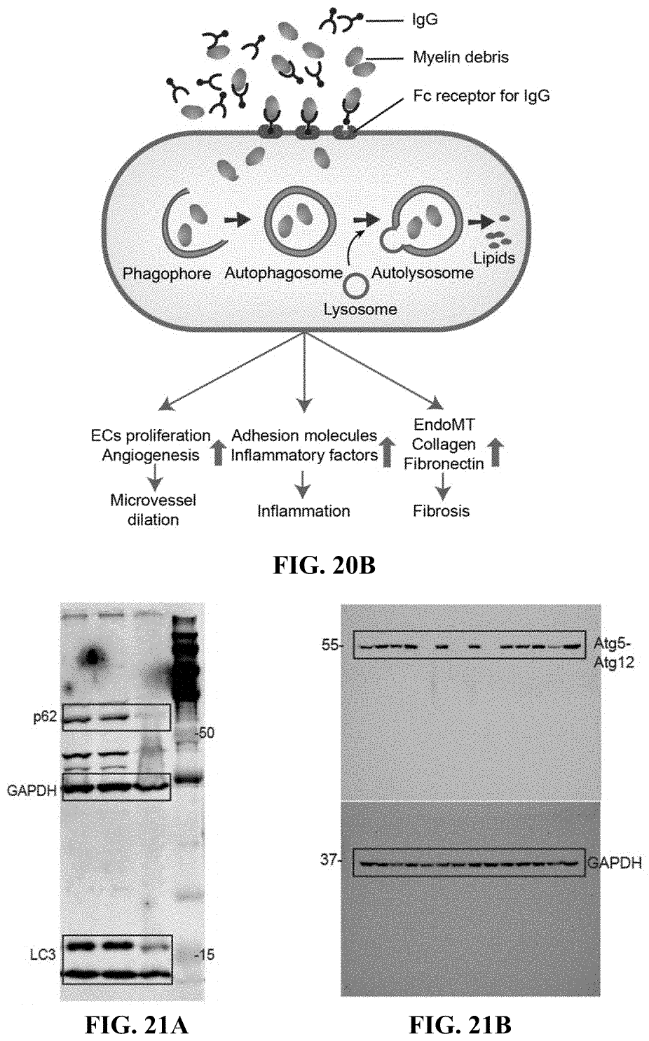

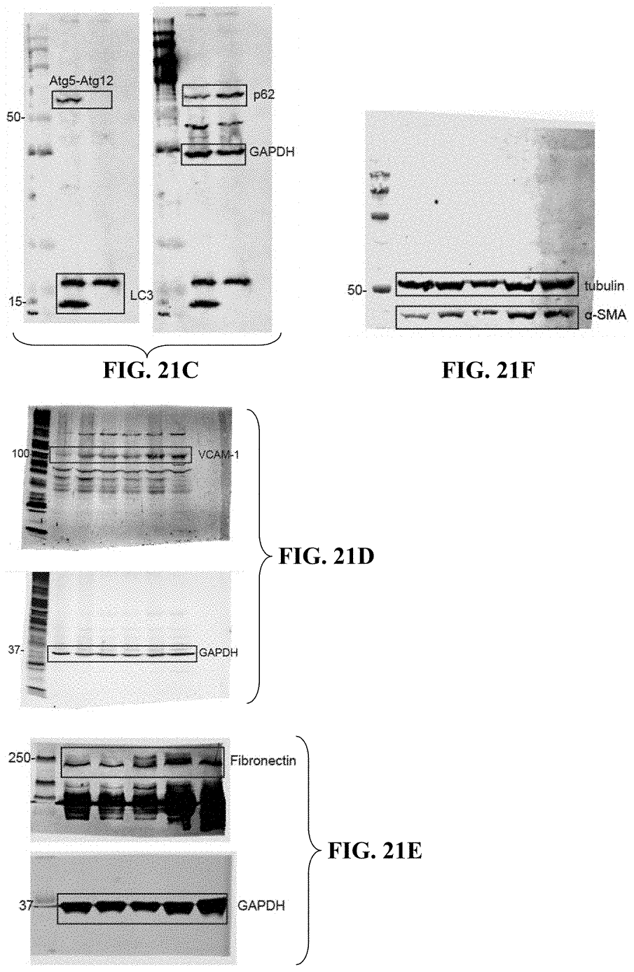

Engulfed myelin debris is delivered through autophagosomes to lysosomes for myelin degradation to lipids in BMECs. (FIGS. 4A, 4A-1) Lysosomes stained with Lysotracker Red dye (red) in BMECs treated with or without CFSE-myelin debris (green) for 72 hr. The zoomed images (FIG. 4A-1) show the detailed size and spatial relationship of lysosomes and engulfed myelin debris in naïve-BMECs and myelin-BMECs. Arrowheads indicate lysosomes containing no myelin debris, arrows indicate lysosomes containing engulfed myelin debris. Scale bar, 10 μm (FIG. 4A), 1 μm (FIG. 4A-1). (FIG. 4B) Quantification of the size of lysosomes in naïve BMECs and myelin-BMECs. Data are shown as means±s.e.m. p<0.0001 (****) by unpaired Student's t-test. (FIG. 4C) Quantification of LC3⁺ puncta per cell in BMECs cultured with 5% serum (fed), after starvation for 6 hr (starved), after treatment with myelin debris for 72 hr (myelin-ECs). (FIG. 4D, 4D-1) Representative confocal images (FIG. 4D) and 3D view (FIG. 4D-1) of co-localization between CFSE-myelin puncta (green) and LC3⁺ or GABARAP⁺ autophagosomes (red), between myelin puncta (green) and Rab5⁺ early endosomes (red), or Rab7⁺ late endosomes (red). Scale bar, 1 μm. Immunoblot for LC3 and p62 in BMECs treated with myelin debris for 0, 24 hr and 72 hr in the presence or absence of 10 nM Bafilomycin A1 for 24 hr. Myelin-ECs induced a significant increase of autophagosome formation (LC3b-puncta), while BMECs fed with sufficient nutrient showed very few LC3b-puncta (negative control), and nutrient withdrawal by starvation induced robust formation of LC3b-puncta (positive control) (FIG. 4E). Quantification of protein level of LC3-II (FIG. 4F-1) and p62 (FIG. 4F-2) determined by densitometry analysis relative to tubulin. Data are shown as means±s.e.m. from 3 independent immunoblots. p<0.05 (* or ^{#}), p<0.01 (**) by ANOVA analysis of assays with or without Bafilomycin A1 treatment, followed by Student's t-test. The Atg5 knockout BMECs failed to generate LC3⁺ puncta (FIG. 4H) and induced LC3 conversion (FIG. 4I), as well as accumulated p62 and ubiquitin (FIGS. 4I, 15C), verifying the knockout is functional. (FIG. 4J) Myelin degradation into neutral lipids (stained by ORO) in myelin-BMECs followed by treatment with 10 nM Bafilomycin A1, 1 mM 3-MA, 1 μM rapamycin for the indicated time points. Scale bar, 20 (FIG. 4K) Quantification of ORO⁺ lipids in (FIG. 4J) as well as in myelin-BMECs after 24 hr culture in 0% FBS medium (starvation). Data are shown as means±s.e.m. from 3 independent experiments. p<0.05 (*), p<0.0001 (***) by Student's t-test.

FIGS. 5A, 5B, 5C, 5C-1, 5D, 5D-1, 5E, 5F, 5F-1, 5G, 5H, 5I, 5J, and 5K. Myelin debris uptake contributes to angiogenesis in SCI and EAE. (FIG. 5A) Microvessels (CD31, red) in three different regions, classified as uninjured, marginal and injured regions (see methods for detailed classification) in an injured spinal cord from 1-week SCI mouse. Scale bar, 500 μm; 50 μm in zoomed images. (FIG. 5B) Corresponding quantification of microvessel diameter in the normal spinal cord, uninjured region, marginal regions and injured regions from mice after 1-, 4-, 6-, 8- and 10-week of SCI. The number of mice analyzed is: normal (n=5); 1-week (n=5); 4-week (n=4); 6-week (n=4); 8-week (n=4); 10-week (n=3). (FIG. 5C) Immunostaining for Ki-67 (green) and CD31 (red) in normal spinal cord from uninjured mice and injured spinal cord from 7 daySCI mice. The zoomed images showed the Ki-67+ microvessels. Arrowheads indicate Ki-67+ ECs. Scale bar, 50 μm (upper images), 10 μm (lower images). (FIG. 5C-1) Corresponding quantification of Ki-67+ ECs in normal spinal cords from uninjured mice and injured spinal cords from SCI mice. Data are shown as means±s.e.m. Normal mice (n=4) and SCI mice (n=5). p=0.0029 (**) by unpaired Student's t-test. (FIG. 5D) Microvessels (CD31, red) and myelin (MBP, green) staining in 15-day EAE spinal cord. Arrowheads indicate enlarged microvessels in demyelinated regions (MBP negative). Scale bar, 50 μm. 20 μm in zoomed images to the right. (FIG. 5D-1) Quantification of the diameter of microvessels in non-demyelinated and demyelinated regions from 7-day and 15-day EAE spinal cords. Data are shown as means±s.e.m. from 3 mice. 7-day, p=0.0107 (*); 15-day, p=0.0139 (*) by paired Student's t-test. (FIG. 5E) Immunostaining for Ki-67 (green) and CD31 (red) in spinal cord from 15 day EAE mouse. Arrowheads indicate Ki-67+ ECs on microvessels. Scale bar, 20 μm. (FIGS. 5F, 5F-1) Quantification of Ki-67+ cells in primary BMECs and bEnd.3. cell line treated with myelin debris for 72 hr or the indicated time points. Data are shown as means±s.e.m. naïve ECs (n=4), myelin-ECs (n=3). Primary ECs, p=0.026 (*). Cell line, 72 hr, p=0.0226 (*); 96 hr, p=0.0118 (*); 120 hr, p=0.001 (***) by unpaired Student's t-test. (FIG. 5G) Number of BMECs treated with myelin debris for the indicated time points. Data are shown as means±s.e.m. from 3 independent assays. 72 hr, p=0.0026 (**), 96 hr, p<0.0001 (****); 120 hr, p=0.0079 (**) by unpaired Student's t-test. (FIG. 5H) Gross images of Matrigel plugs injected with PBS, naïve ECs and myelin-ECs in normal mice. Insets are CD31 immunostaining of matrigel slices. Scale bar in insets, 20 μm. The bottom showed the corresponding quantification of CD31+ microvessels in each Matrigel plug. Data are shown as means±s.e.m. from 3 independent Matrigel plugs. Myelin-ECs vs naïve ECs, p=0.0002 (***) by unpaired Student's t-test. (FIG. 5I) Cell number of wild-type or Atg5 knockout BMECs with or without myelin debris treatment for 72 hr. Data are shown as means±s.e.m. from 3 assays. p=0.0401 (*) by unpaired Student's t-test. (FIG. 5J) qRT-PCR analysis of gene expression of VEGF in naïve BMECs and myelin-ECs. Data are shown as means±s.e.m. from 3 biologically independent replicates. p<0.0015 (**) by unpaired Student's t-test. (FIG. 5K) Cell number of BMECs treated with myelin debris in the presence of control IgG or neutralizing VEGF antibody (20 μg/ml, 72 hr). Data are shown as means±s.e.m. (n=3). p=0.61 (ns), p=0.0184 (*) by unpaired Student's t-test.

FIGS. 6A, 6A-1, 6B, 6C, 6C-1, 6D, 6E, 6F, 6F-1, 6G, and 6H. Myelin debris uptake induces endothelial inflammation leading to BMDMΦ infiltration across microvessels. (FIGS. 6A, 6A-1) Representative confocal images (FIG. 6A) and 3-D reconstructed images (FIG. 6A-1) showing the spatiotemporal distribution of bone marrow-derived GFP+ cells (GFP+ BMDCs) and CD31+ microvessels (red) in normal spinal cord and injured spinal cord from GFP+ bone marrow chimeric mice after 3 days, 1 week and 2 weeks of SCI. Scale bar, 20 μm. (FIG. 6B) Representative confocal images and 3-D reconstructed images showing the spatial distribution of Iba-1+ cells relative to microvessels (red) in 1-week mouse EAE spinal cord. Scale bar, 20 μm. (FIG. 6C) Representative images showing adhered BMDMΦ (Mac-2, red) on the monolayer of naïve-ECs or myelin-ECs (DIC imaging, grey). Scale bar, 100 μm. (FIG. 6C-1) Corresponding quantification of adhered BMDMΦ on BMECs in (FIG. 6C) and Atg5 knockout BMECs as well as wild-type BMECs cultured with 70° C. heated serum (that is, no IgG or IgG-inactivated serum), the value was shown as the percentage of adhered BMDMΦ relative to the underlying BMECs. Data were presented as means±s.e.m. (n=3). p=0.0023 (**); p=0.0072 (##); p=0.0129 (#) by unpaired Student's t-test. (FIG. 6D) Immunoblot and quantification of VCAM-1 expression in BMECs treated with myelin debris for the indicated time points. Quantification of VCAM-1 expression level was determined by densitometry analysis relative to GAPDH. Data were presented as means±s.e.m. (n=3). 1d, p=0.024 (*); 2d, p=0.0008 (***); 3d, p=0.0013 (**); 4d, p=0.0011 (**); 5d, p=0.0012 (**) by unpaired Student's t-test. Uncropped blots are in FIG. 21D. (FIG. 6E) Images of migrated BMDMΦ (violet crystal staining) towards conditioned media from ECs. BMDMΦ were added to the upper chamber and allowed to migrate through the membrane into the lower chamber containing conditioned media from naïve-ECs or myelin-ECs. (FIGS. 6F, 6F-1) ELISA detection for chemokine MCP-1 secreted by naïve-ECs or myelin-ECs in primary cultures (FIG. 6F) or cell line (FIG. 6F-1). Data were showed as means±s.e.m. n=3. p=0.0006 (***), p=0.0029 (**) by unpaired Student's t-test. (FIG. 6G) Quantitative RT-PCR analysis of IL-6 in BMDMΦ treated with conditioned media from naïve-ECs or myelin-ECs. Data were presented as means±s.e.m. (n=3). p=0.0003 (***) by unpaired Student's t-test. (FIG. 6H) Immunostaining of macrophages/microglia (Iba-1, green) and astrocytes (GFAP, red) in normal spinal cords injected with CFSE-labeled naïve ECs or myelin-ECs (white). Scale bar, 100 μm, 10 μm (inset images).

FIGS. 7A, 7B, 7C, 7D, 7E, 7F, 7G, 7G-1, 7H, and 7I. Myelin debris engulfment promotes endothelial deposition of pro-fibrotic components. (FIGS. 7A, 7B, 7C) Immunostaining for collagen I (green) and CD31 (red) in normal spinal cords (FIG. 7A) and in the lesion cores from mice after 6 weeks of SCI (FIG. 7B) and in 15-day mouse EAE spinal cord (FIG. 7C). (FIGS. 7D, 7E, 7F) Immunostaining for fibronectin (green) and CD31 (red) in normal spinal cords (FIG. 7D) and in the lesion cores from mice after 6 weeks of SCI (FIG. 7E) and in 15-day mouse EAE spinal cord (FIG. 7F). Scale bar, 500 μm (upper images), 20 μm (lower zoomed images). (FIG. 7G) Immunostaining of collagen I (green) and CD31 (red) in BMECs treated with or without CFSE-myelin debris (pseudo white) for 10 days or treatment with 10 ng/ml of TGF-β1 for 3 days. Scale bar, 20 μm. (FIG. 7G-1) Quantification of collagen I fluorescent intensity in (FIG. 7G), Data are shown as means±s.e.m. (n=3). Myelin vs control, p=0.0301 (*); TGF-β1 vs control, p=0.0159 (*) by ratio Student's t-test. (FIG. 7H) Immunostaining of collagen I (green) in BMECs-induced microvessels-like structures (CD31, red) treated with or without CF SE-myelin debris (pseudo white) for 72 hr. Scale bar, 100 μm. (FIG. 7I) Immunoblot for fibronectin in BMECs at baseline or after treatment with myelin debris for the indicated time points. Treatment with 10 ng/ml of recombinant TGF-β1 for 5 days was used as positive control. Corresponding quantification of protein level was determined by densitometry analysis relative to GAPDH or tubulin, as shown at the bottom of each blot. The immunoblots are representative of two independent experiments. Uncropped blots are shown in FIG. 21E.

FIGS. 8A, 8B, 8C, 8C-1, 8D, 8E, 8F, 8G, and 8H. Myelin debris engulfment induces endothelial-to-mesenchymal transition (endoMT). (FIG. 8A) Phase contrast images of wild-type BMECs and Atg5 knockout BMECs with the indicated treatment: TGF-β1 (10 ng/ml, 3 days), myelin debris (1 mg/ml, 10 days), myelin+ pan-TGF-β neutralizing antibody (20 μg/ml, 10 days). The insets showed the magnified views of cell morphology. Scale bar, 100 μm. (FIG. 8B) Immunostaining of α-SMA (green) and CD31 (red) of wild-type BMECs and Atg5 knockout BMECs with the indicated treatment as above, myelin debris is shown in pseudo white. The zoomed images below showed the detailed immunostainings of a-SMA, CD31 and myelin debris. Scale bar, 20 μm (upper images), 10 μm (lower images). (FIG. 8C) Quantification of spindle-shaped BMECs. Data are shown as means±s.e.m. (n=3). p=0.0008 (***), p=0.0027 (**) vs control, p=0.0029 (##), p=0.001 (###) vs wild-type myelin group, analysis by unpaired Student's t-test. FIG. 8C-1 shows the criteria for spindle-shape cells. (FIG. 8D) Quantification of α-SMA+CD31+ BMECs. Data are shown as means±s.e.m. (n=3). TGF-β1 vs control, p=0.0015 (**); myelin vs control, p=0.0063 (**); myelin+TGF-β Ab vs myelin, p=0.0094 (##); myelin+Atg5 KO Ab vs myelin+Wild-type, p=0.0063 (##), analysis by unpaired Student's t-test. (FIG. 8E) Quantitative RT-PCR analysis of gene expression of a-SMA in naïve ECs and myelin-ECs. Data are shown as means±s.e.m. from 3 biologically independent replicates. p=0.03 (*) by unpaired Student's t-test. (FIG. 8F) Immunoblot for a-SMA in BMECs at baseline or after treatment with myelin debris for the indicated time points. Treatment with 10 ng/ml of recombinant TGF-β1 for 5 days was used as positive control. Corresponding quantification of protein level was determined by densitometry analysis relative to GAPDH or tubulin, as shown at the bottom of each blot. The immunoblots are representatives of two independent experiments. Uncropped blot is in FIG. 21F. (FIG. 8G) Quantitative RT-PCR analysis of gene expression of TGF-β1 in naïve ECs and myelin-ECs. Data are shown as means±s.e.m. from 3 biologically independent replicates. p=0.015 (*) by unpaired Student's t-test. (FIG. 8H) Immunostaining for a-SMA (green) and CD31 (red) in normal spinal cords and in the lesion cores from mice after 6 weeks of SCI and 15 days of EAE. Scale bar, 20 μm (images to the left), 5 μm (zoomed images to the right).

FIG. 9. Internalization of myelin debris (MBP, green) by microvessels (CD31, red) in injured and uninjured regions of spinal cords from different time points of SCI. The x-y and x-views show myelin debris was internalized by microvessels in injured regions of spinal cords from 1-day, 3-day and 5-day SCI mice. Scale bar, 20 μm.

FIGS. 10A and 10B. MOG-induced EAE disease phases and behavioral assessment. (FIG. 10A) The graph shows disease progression through the 3 stages described. Arrows indicate the number of animals used for the assessment up until the specified sacrifice date. Following MOG35-55/CFA immunization but before onset, C57BL/6J mice do not display any motor deficits (pre-onset stage), however several days later, at the onset of disease the tail becomes weak or flaccid and the mice start showing hind limb weakness (onset stage). After disease onset, the mice progressively lose hind limb function and may even develop forelimb weakness consequent to hind limb paralysis (disease stage), with a disease peak occurring relatively soon after onset. (FIG. 10B) Immunostaining for MBP (red) and nucleus (blue) spinal cord lumbar segment and thoracic segment after 15-day EAE. The arrowheads indicate the demyelinated plaques. Lumbar segment has more severe demyelination than thoracic segment. Scale bar, 500 μm.

FIG. 11. BMDMΦ engulf myelin debris rapidly. Detection of engulfed CFSE-myelin debris (green) in BMDMΦ (F4/80, red) exposed to myelin debris for 0, 15 min, 1 hr and 3 hr, respectively. Scale bar, 5 μm.

FIGS. 12A and 12B. CR3 or Mac2 is involved in BMDMΦ engulfment of myelin debris (FIG. 12A), while not involved in ECs engulfment of myelin debris (FIG. 12B). Detection of myelin debris (green) engulfment by BMDMΦ and BMECs that were pre-treated with the indicated neutralizing antibodies for 3 hr, followed by myelin debris treatment for 3 hr and 72 hr, respectively. A combined use of CR3 and Mac2 neutralizing antibody blocked BMDMΦ uptake of myelin debris, but did not block BMEC uptake of myelin debris. Scale bar, 100 μm.

FIG. 13. Deficiency of MBP, a proposed ligand for LRP-1 receptor appears not affect myelin debris uptake by BMECs. Detection of engulfed CFSE-myelin debris (CFSE, green or MBP, red) in BMECs (CD31, purple) exposed to myelin debris (isolated from wild-type mice) and MBP-deficient myelin debris (isolated from MBPshi/shi knockout mice) for 72 hr. Scale bar, 20 μm.

FIG. 14. Serum-dependent engulfment of myelin debris by BMECs. Detection of engulfed CFSE-myelin debris (green) in BMECs after 48 hr exposure to myelin debris in the presence of 0%, 1% and 5% FBS. Scale bar, 200 μm.

FIGS. 15A-15D. Generation and characterization of Atg5 knockout BMEC cell line. (FIG. 15A) Diagram showing Atg5 gene locus and guide RNA targeting sequence (SEQ ID NO:23). (FIG. 15B) Verification of genomic editing of Atg5 by CRISPR-Cas9 system using T7E1 assay. T7E1 assay was run in acrylamide gel, which caused inaccurate indication of 100 bp DNA ladder. The PCR product band is 440 bp. Two digestion bands are 228 and 212 bp, which are indistinguishable from each other in the gel. (FIG. 15C) Functional verification of Atg5 knockout BMECs by immunostaining of poly-ubiquitin (green) and p62 (red). Scale bar, 20 μm. (FIG. 15D) Cell death analysis of Atg5 knockout BMECs and drugs-treated wild-type BMECs by propidium iodide (PI) staining. The drug dose and incubation period correspond to that of FIG. 4K. H₂O₂ treatment was included as positive control. Data are shown as means±s.e.m. (n=3). p<0.0001 (****) by unpaired Student's t-test.

FIGS. 16A, 16B, and 16B-1. Additional data on in vivo angiogenesis. (FIG. 16A) Microvessels (CD31, red) in normal spinal cord from uninjured mice and injured spinal cords from mice after 1 week or 6 weeks of SCI. The insets showed the enlarged images. Scale bar, 200 μm; 20 μm in inset images. (FIGS. 16B, 16B-1) Representative images (FIG. 16B) and quantification (FIG. 16B-1) of formation of Ki-67 positive (green) and CD31 (red) microvessels after the indicated injections into normal spinal cords in mice. Scale bar, 100 μm; 50 μm in zoomed images. Data are shown as means±s.e.m. from 3 injected mice. Myelin vs PBS, p=0.6779 (ns); naïve ECs vs PBS, p=0.0474 (*); myelin-ECs vs PBS, p=0.0004 (***); myelin-ECs vs naïve ECs, p=0.0005 (***); myelin-ECs (IgG-inactivated) vs myelin-ECs, p=0.0005 (***) by unpaired Student's t-test.

FIGS. 17A-17H. The cellular consequences of necrotic neuronal cell bodies or zymosan engulfment by BMECs and BMDMΦ. (FIG. 17A) Characteristic morphology of neuronal cells that were differentiated from N2A cells, a mouse neuroblastoma cell line. Scale bar, 50 μm. (FIG. 17B) BMECs (red) engulfment of necrotic neuronal cell body (green) for 72 hr. BMEC nuclei (Hoechst staining) were shown in pseudo white. Arrowhead indicated the squeezed and distorted BMECs nucleus by the engulfed dead cell. Scale bar, 10 μm. (FIG. 17C) Quantification of the percentage of BMDMΦ and BMECs that contained necrotic neuronal cell bodies after 6 hr and 72 hr engulfment, respectively. Data are shown as means±s.e.m. from 3 biologically independent replicates. p=0.0006 (***) by unpaired Student's t-test. (FIGS. 17D, 17E, 17F) Proliferation (FIG. 17D), inflammation (FIG. 17E) and fibrosis-related (FIG. 17F) change of BMECs with or without engulfment of necrotic neuronal cell bodies by counting BMEC nucleus number, by q-PCR analysis of MCP-1 gene expression and by analyzing the percentage of a-SMA+CD31+ cells, respectively. Data are shown as means±s.e.m. from 3 biologically independent cultures. FIG. 17D, p=0.0418 (*); FIG. 17E, p=0.9763 (ns); FIG. 17F, p=0.5369 (ns) by unpaired Student's t-test. FIGS. 17G and 17H show engulfment of pathogen by endothelial cells and macrophages. Zymosan, which could be efficiently engulfed by BMDMΦ (FIG. 17G), could not be engulfed by BMECs (FIG. 17H).

FIGS. 18A-18C. Analysis of astrocytes effects on BMECs using a co-culture system. (FIG. 18A) Primary mouse astrocytes were isolated and characterized by GFAP staining (green). (FIG. 18B) Diagram showing astrocytes-BMECs co-culture in a transwell. (FIG. 18C) BMEC proliferation analysis after co-cultured with the indicated astrocytes. Myelin-treated BMEC was included as positive control. Data are shown as means±s.e.m. from 3 biologically independent cultures. Control astrocytes vs medium, p=0.0712 (ns); LPS-treated astrocytes vs medium, p=0.1566 (ns); myelin debris-treated ECs vs medium, p=0.0466 (*) by unpaired Student's t-test.

FIGS. 19A-19F. Additional data on inflammation. (FIG. 19A) Quantification of number of GFP+ BMDCs that were associated with microvessels shown in FIGS. 6A and 6A-1. Data are shown as means±s.e.m. (n=3). p=0.0043(**), p=0.0003 (***), p<0.0001 (****) by unpaired Student's t-test. (FIG. 19B) Quantification of number of Iba1+ cells associated with one microvessel with normal size or increased size in FIG. 6B. Data are shown as means±s.e.m. (n=3). p=0.0114 (*) by paired Student's t-test. (FIG. 19C) Representative images showing adhered BMDMΦ (Mac-2, red) on BMECs with the indicated treatments. Scale bar, 100 μm. (FIG. 19D) Representative images of Iba-1 (green) and GFAP staining (red) after the indicated injections into normal spinal cords in mice. Scale bar, 100 μm; 10 μm in inset images. (FIG. 19E) Quantification of Iba-1 fluorescent intensity shown in FIG. 19D. Data are shown as means±s.e.m. (n=3). Myelin debris vs PBS, p=0.0527 (ns); naïve ECs vs PBS, p=0.1942 (ns); myelin-ECs vs PBS, p=0.0038 (**); myelin-ECs vs naïve ECs, p=0.0286 (*); myelin-ECs vs myelin debris, p=0.0067 (**); myelin-ECs (IgG-inactivated) vs myelin-ECs, p=0.0279 (*) by unpaired Student's t-test. (FIG. 19F) Quantification of GFAP fluorescent intensity shown in FIG. 19D. Data are shown as means±s.e.m. (n=3). Myelin debris vs PBS, p=0.1523 (ns); naïve ECs vs PBS, p=0.3266 (ns); myelin-ECs vs PBS, p=0.0183 (*); myelin-ECs vs naïve ECs, p=0.0349 (*); myelin-ECs vs myelin debris, p=0.044 (*); myelin-ECs (IgG-inactivated) vs myelin-ECs, p=0.0336 (*) by unpaired Student's t-test.

FIGS. 20A and 20B. Schematic diagram depicting the novel function of microvascular ECs in engulfment and autophagic processing of myelin debris, which in turn boosts secondary injury by promoting inflammation, microvessel dilation and fibrotic scar formation. (FIG. 20A) In the injured spinal cord, one of the immediate responses to SCI is the fragmentation of myelin sheaths into myelin debris, which can be quickly cleared by professional phagocytes such as BMDMΦ and microglia. We demonstrated that microvessels and the lining microvascular ECs are able to engulf myelin debris which promote chronic inflammation and pathological healing. The microvessels in SCI injury core are dilated (not depicted), in close vicinity to BMDMΦ, deposit high levels of pro-fibrotic components like collagen and fibronectin. These changes in the injury core, all of which could be stemmed as a result of microvascular engulfment of myelin debris, are closely associated with the progression of SCI. (FIG. 20B) Mechanisms for myelin debris entry, processing and actions in microvascular ECs. IgG opsonization of myelin debris is engulfed by ECs through Fc receptor. Engulfed myelin debris is delivered through autophagosomes to lysosomes for myelin degradation into neutral lipids. Functionally, myelin debris uptake promotes endothelial cells proliferation/angiogenesis, endothelial inflammation and endothelial deposition of pro-fibrotic components, which could possibly contribute to the dilation of microvessels, chronic inflammation and fibrotic scar formation seen in injury core.

FIGS. 21A-21F. Uncropped immunoblots for FIGS. 4F (FIG. 21A), 4G (FIG. 21B), 4I (FIG. 21C), 6D (FIG. 21D), 7I (FIG. 21E), and 8F (FIG. 21F).

### BRIEF DESCRIPTION OF THE SEQUENCES

SEQ ID NOs: 1-22 are forward and reverse primers used for quantitative real-time polymerase chain reaction (RT-PCR).

SEQ ID NO:23 is the sequence of the Atg5 gene locus (FIG. 15A), containing a guide RNA targeting sequence (SEQ ID NO:24).

SEQ ID NO:24 is a single guide RNA targeting sequence.

### DETAILED DESCRIPTION OF THE INVENTION

The inventor hypothesized that in spinal cord injury (SCI) microvessels and the lining microvascular ECs serve as amateur phagocytic sources for myelin debris uptake, which would be consistent with the early presence and great number of newly formed microvessels in the injury core.

In the study described herein, the inventor established a previously unidentified role for microvessels and lining microvascular ECs in engulfing and clearing myelin debris after SCI and experimental autoimmune encephalomyelitis (EAE), a commonly used animal model of multiple sclerosis (MS). The inventor also discovered a novel pathway for myelin debris degradation through the autophagy-lysosome system. Importantly, the inventor demonstrated for the first time that microvascular EC uptake exerts critical functions beyond myelin debris clearance. Engulfment and autophagic processing of myelin debris by microvascular ECs have sequential consequences in promoting chronic inflammation and pathological healing (angiogenesis and fibrotic scar formation) during the progression of demyelinating disorders. Therefore, this study reveals how myelin debris engulfment and processing by microvascular ECs contribute to pathological progression in demyelinating disorders.

The inventor determined that microvessels and lining ECs act as amateur phagocytes to engulf myelin debris generated by disorders associated with demyelination. The inventor determined that IgG opsonization of myelin debris is required for efficient uptake by microvascular ECs. The engulfed myelin debris is then delivered through the autophagy-lysosome pathway for intracellular degradation. Functionally, engulfment and autophagy-dependent processing of myelin debris by microvascular ECs contribute to three critical processes that are closely associated with demyelinating disorders: robust angiogenesis that results in excessive and abnormal microvessels, chronic inflammation, and endothelial-mediated fibrosis that most likely takes place through endoMT. Therefore, the inventor proposes that a benefit to patients can be obtained by intervening and interfering with the effects of myelin-ECs by targeting these particular processes (e.g., myelin debris uptake, autophagy and endoMT).

In the context of spinal cord injury, it was observed that vascular endothelial cells (VECs), through non-canonical phagocytic mechanisms, are capable of endocytosing myelin debris produced by damaged nerve tissue. In doing so, these VECs appear to promote fibrosis by becoming fibroblast-like cells. Fibrosis resulting from nervous tissue is generally viewed as deleterious. The non-professional phagocytic activity of VECs appears to be conducted through autophagy-linked molecular pathways, including the protein, ATG5. ATG5 is a well-characterized, critical molecular component in normal cell autophagy.

The data described herein support the use of directed interference of autophagy pathways, e.g., by ATG5 inhibition, to treat, prevent, or delay onset of a demyelinating condition, and to reduce VEC-induced fibrosis, such as fibrotic scarring that occurs following neural injury such as spinal cord injury. The data further suggest that fibrotic VEC phagocytosis is also dependent on immunoglubulin G (IgG); therefore, immunodepletion of IgG can be used to reduce VEC-induced fibrosis in a manner similar to targeted ATG5 inhibition. The invention concerns methods for using one or both approaches to reduce fibrosis, or to treat a fibrotic condition in a subject.

The invention concerns a method for treating a demyelinating condition in a human or animal subject, wherein the method comprises administering an agent to the subject that inhibits vascular endothelial cell phagocytosis. In some embodiments, the demyelinating condition is associated with a wound or injury, such as a neural injury (an injury to nervous tissue). For example, the neural injury may be a neuropraxia, axonotmesis, or neurotmesis. The neural injury may be an injury nervous tissue of the peripheral nervous system (PNS), central nervous system (CNS), or both. For example, the injury may be a spinal cord injury (SCI).

The invention also concerns agents that inhibit vascular endothelial cell phagocytosis for use in treatment of demyelination and demyelinating conditions, and use of an agent that inhibits vascular endothelial cell phagocytosis in the manufacture of a medicament for the treatment of demyelination or a demyelinating condition.

The demyelinating condition may associated with a wound, i.e., a disruption of normal continuity of physiological structures. The wound may be any type, acute or chronic, of any cause, such as physical, chemical, or radiation. For example, the wound may be caused by trauma or surgery, or a burn. Common types of wounds include acute wounds, skin tears, shear damage, and pressure damage.

In some embodiments, the wound is a wound or other injury of the nervous tissue of the peripheral nervous system (PNS), central nervous system (CNS), or both. For example, the wound may be a spinal cord injury (SCI) or traumatic brain injury (TBI). The SCI may be in any location of the spinal cord (cervical, thoracic, lumbar, sacral), and may be any type severity of SCI (complete or incomplete), such as anterior cord syndrome, central cord syndrome, or Brown-Sequard syndrome. In some embodiments, the wound is a CNS wound caused by one or more of the following: trauma, surgery, ischemia-reperfusion injury, chemotherapy-induced injury, radiotherapy-induced injury, infection, and the body's immune system.

Among other physiological effects, the method of the invention may be used to reduce or inhibit one or more of: formation or deposition of tissue fibrosis; reducing the size, cellularity (e.g., fibroblast or immune cell numbers), composition; or cellular content, of a fibrotic lesion; or reducing fibrosis associated with an inflammatory response.

Thus, by extension, the method for treating a demyelinating condition may reduce fibrosis, or treatment of a fibrotic condition in a human or animal subject. The fibrosis or fibrotic condition may be one that is vascular endothelial cell-induced, i.e., mediated by vascular cell phagocytosis (endocytosis of myelin debris by vascular endothelial cells through non-canonical phagocytic mechanisms).

The fibrotic condition may be a primary fibrosis. The fibrotic condition may be idiopathic. In some embodiments, the fibrotic condition is associated with (e.g., is secondary to) a disease (e.g., an infectious disease, an inflammatory disease, an autoimmune disease, a malignant or cancerous disease, and/or a connective disease); a toxin; an insult (e.g., an environmental hazard (e.g., asbestos, coal dust, polycyclic aromatic hydrocarbons), cigarette smoking, a wound); a medical treatment (e.g., surgical incision, chemotherapy or radiation), or a combination of any of the foregoing. In some embodiments, the fibrotic condition is associated with an autoimmune disease selected from scleroderma or lupus, e.g., systemic lupus erythematosus.

It may be desirable to use the method of the invention in a surgical setting, such as peritoneal, pericardial, obstetric, gynecological, neurosurgical, arthroscopic, orthopedic, plastic, reconstructive, muscle, or tendon surgery. Specific examples of surgery where it would be advantageous to administer the agent that inhibits vascular endothelial cell phagocytosis before, during, and/or after surgery include abdominal surgery, joint surgery, tendon surgery, surgery to remove pelvic sidewall adhesions, peritoneal surgery, thoracic surgery, vascular surgery, cardiac surgery, heart bypass surgery, heart valve replacement surgery, or open heart surgery, laminectomy, fallopian tube surgery, plastic surgery, arthritis & osteoarthritis, and surgery to treat temporo-mandibular joint dysfunction. The agent may be administered before, during, or after any procedure in which there is a risk of inducing a demyelinating condition, such as a surgery in which neural injury may occur.

In some embodiments, the subject has a demyelinating condition, e.g., a condition that results in damage to the myelin sheath surrounding the spinal cord, brain, optic nerve, or any combination thereof, interfering with the normal conduction of signals in the affected nerves.

As used herein, a “demyelinating condition” is a condition that destroys, or disrupts the integrity of, or damages, a myelin sheath. As used herein, the term “myelin sheath” refers to an insulating layer surrounding vertebrate peripheral neurons increases the speed of conduction and is formed by Schwann cells in the peripheral nervous system or by oligodendrocytes in the central nervous system. In some embodiments, the demyelinating condition is caused by an injury to neural tissue. In some embodiments, the demyelinating condition is hereditary. Some examples of demyelinating conditions are described in Love S, “Demyelinating diseases”, J Clin Pathol, 2006, 59:1151-1159).

Examples of demyelinating conditions include, but are not limited to, spinal cord injury, traumatic brain injury, multiple sclerosis (MS), Alzheimer's disease, autoimmune encephalomyelitis, acute disseminated encephalomyelitis (ADEM), Balo's disease (concentric sclerosis), Charcot-Marie-Tooth disease (CMT), Guillaian Barre Syndrome (GBS), HTLV-1-associated myelopathy (HAM), neuromyelitis optica (Devic's disease), Schilder's disease, transverse myelitis, congenital metabolic disorder with demyelination, neuropathy with abnormal myelination, drug-induced demyelination, radiation-induced demyelination, hereditary demyelination condition, prion-induced demyelination, encephalitis-induced demyelination, and chronic inflammatory demyelinating neuropathy.

Examples of chronic inflammatory demyelinating neuropathies include, but are not limited to, chronic Immune Demyelinating Polyneuropathy (CIDP); multifocal CIDP; multifocal motor neuropathy (MMN); anti-MAG Syndrome (Neuropathy with IgM binding to Myelin-Associated Glycoprotein); GALOP Syndrome (Gait disorder Autoantibody Late-age Onset Polyneuropathy); anti-sulfatide antibody syndrome; anti-GM2 gangliosides antibody syndrome; POEMS syndrome (Polyneuropathy Organomegaly Endocrinopathy or Edema M-protein Skin changes); perineuritis; and IgM anti-GD1b ganglioside antibody syndrome.

The demyelinating condition may be any form or severity. For example, in the case of MS, the condition may be clinically isolated syndrome (CIS), relapsing-remitting multiple sclerosis (RRMS), secondary progressive multiple sclerosis (SPMS), or primary progressive multiple sclerosis (PPMS).

The method of the invention involves administering an agent or treatment to the subject that inhibits vascular endothelial cell phagocytosis in the subject. In some embodiments, the agent or treatment inhibits the autophagy-lysosome pathway in vascular endothelial cells of the subject. In some embodiments, the agent that inhibits the autophagy-lysosome pathway is an agent that inhibits ATG5 in vascular endothelial cells.

Examples of agents that inhibit the autophagy-lysosome pathway include, but are not limited to, a MAP kinase inhibitor (e.g., SP600125, U0126, SB202190, and SB203580), PI3K inhibitor (e.g., 3-methyladenine, LY294002, and Wortmannin), protein biosynthesis inhibitor (e.g., cycloheximide), Vacuolar-type H (+)-ATPase (V-ATPase) inhibitor (e.g., bafilomycin), lysosomal lumen alkalyzer (e.g., chloroquine, hydroxychloroquine, NH4Cl, neutral red, Lys01,and Lys05), acid protease inhibitor (e.g., leupeptin, E64d, and pepstatin A), and endosome inhibitor (e.g., Bafilomycin A1, and chloroquine).

Additional agents that inhibit the autophagy-lysosome pathway include, but are not limited to, 3-methyladenine (3-MA), CPD18 (a.k.a. 3-methyl-6-(3-methylpiperidin-1-yl)-3H-purine), bafilomycin A1, chloroquine, hydroxychloroquine, LY294002 (a.k.a. 2-(4-Morpholinyl)-8-phenyl-4H-1-benzopyran-4-one), SB202190, SB203580, SC79, Wortmannin (a.k.a. SL-2052), SP600125 (a.k.a. 1,9-Pyrazoloanthrone), U0126 (a.k.a. (2Z,3Z)-2,3-bis[amino-(2-aminophenyl)sulfanylmethylidene]butanedinitrile), MHY1485 (a.k.a. 4,6-dimorpholino-N-(4-nitrophenyl)-1,3,5-triazin-2-amine), autophinib, azithromycin, (±)-Bay K 8644, concanamycin A (a.k.a. folimycin), DBeQ (a.k.a. N2,N4-Bis(phenylmethyl)-2,4-quinazolinediamine), E 64d (a.k. a. (2 S,3 S)-3-[ [[(1 S)-3-Methyl-1-[[(3-methylbutyl)amino]carbonyl]butyl]amino]carbonyl]-2-oxiranecarboxylic acid ethyl ester), edaravone (a.k.a. MCI 186), GW 4064 (a.k.a. 3-[2-[2-Chloro-4-[[3-(2,6-dichlorophenyl)-5-(1-methylethyl)-4-isoxazolyl]methoxy]phenyl]ethenyl]benzoic acid), Mdivi 1 (a.k.a. 3-(2,4-Dichloro-5-methoxyphenyl)-2,3-dihydro-2-thioxo-4(1H)-quinazolinone), ML 240 (2-(2-Amino-1H-benzimidazole-1-yl)-8-methoxy-N-(phenylmethyl)-4-quinazolinamine), MRT 67307 (a.k.a. N-[3-[ [5-Cyclopropyl-2-[[3-(4-morpholinylmethyl)phenyl]amino]-4-pyrimidinyl]amino]propyl]cyclobutanecarboxamide), MRT 68601 (N-[3-[[5-Cyclopropyl-2-[[4-(4-morpholinyl)phenyl]amino]-4-pyrimidinyl]amino]propyl]cyclobutanecarboxamide), MRT 68921 (a.k.a. N-[3-[[5-Cyclopropyl-2-[(1,2,3,4-tetrahydro-2-methyl-6-isoquinolinyl)amino]-4-pyrimidinyl]amino]propyl]cyclobutanecarboxamide), NMS 873 (3-[3-(Cyclopentylthio)-5-[[[2-methyl-4′-(methylsulfonyl)[1,1′-biphenyl]-4-yl]oxy]methyl]-4H-1,2,4-triazol-4-yl]pyridine), nocodazole (a.k.a. [5-(2-Thienylcarbonyl)-1H-benzimidazol-2-yl]carbonic acid, methyl ester), pepstatin A, apautin 1 (a.k.a. 6-Fluoro-N-[(4-Fluorophenyl)methyl]-4-quinazolinamine), taxol (a.k.a. paclitaxel), vinblastine (a.k.a. vincaleukoblastine), xanthohumol (a.k.a. (2E)-1-[2,4-Dihydroxy-6-methoxy-3-(3-methyl-2-buten-1-yl)phenyl]-3-(4-hydroxyphenyl)-2-propen-1-one), Tetrahydroacridine 33 (a.k.a. 6-Chloro-N-(1-ethylpiperidin-4-yl)-1,2,3,4-tetrahydroacridin-9-amine), Thapsigargin (a.k.a. 3 S,3aR,4S,6S,6aR,7S,8S,9b S)-6-(Acetyloxy)-4-(butyryloxy)-3,3a-dihydroxy-3,6,9-trimethyl-8-{[(2Z)-2-methylbut-2-enoyl]oxy}-2-oxo-2,3,3a,4,5,6,6a,7,8,9b-decahydroazuleno[4,5-b]furan-7-yl octanoate), ARN5187 (a.k.a. 4(((1-(2-Fluorophenyl)cyclopentyl)-amino)methyl)-2-((4-methylpiperazin-1-yl)methyl)phenol), Spautin-1 (6-fluoro-N-[4-fluorobenzyl]quinazolin-4-amine), N-acetyl cysteine (a.k.a. NAC), L-asparagine (a.k.a. (S)-2-Aminosuccinic acid 4-amide), Catalase from human erythrocytes (a.k.a. H₂O₂:H₂O₂ oxidoreductase), E-64d (a.k.a. (2S,3S)-trans-Epoxysuccinyl-L-leucylamido-3-methylbutane ethyl ester, GMX1778 (a.k.a. N-[6-(4-Chlorophenoxy)hexyl]-N′-cyano-N″-4-pyridinyl-guanidine), Leupeptin (a.k.a. Acetyl-Leu-Leu-Arg-al), and SBI-0206965 (a.k.a. 2-((5-Bromo-2-((3,4,5-trimethoxyphenyl)amino)pyrimidin-4-yl)oxy)-N-methylbenzamide).

In other embodiments, the agent that inhibits the autophagy-lysosome pathway is selected from among an antisense, RNA-interference molecule (e.g., shRNA), and microRNA that targets a component of the autophagy-lysosome pathway (e.g., ATG5) in vascular endothelial cells by blocking or reducing the component's expression, as a genetic intervention.

In some embodiments, the agent that inhibits vascular endothelial cell phagocytosis in the subject is an agent that depletes or inactivates immunoglobulin G (IgG) locally at a desired anatomical site or systemically. For example, an immunomodulating enzyme that hydrolyzes IgG in the subject, such as the endoglycosidase EndoS or the protease IdeS, may be administered to the subject. IdeS cleaves IgG in the lower hinge region, while EndoS hydrolyzes the conserved N-linked glycan in the Fc region (Collin M and Björck L, “Toward Clinical use of the IgG Specific Enzymes IdeS and EndoS against Antibody-Mediated Diseases,” Methods Mol. Biol., 2017, 1535:339-351; Jarnum S et al., “Enzymatic Inactivation of Endogenous IgG by IdeS Enhances Therapeutic Antibody Efficacy”, Mol. Cancer. Therap., 2017 Sep. 16(9):1887-1897; and Winstedt L et al., “Complete Removal of Extracellular IgG Antibodies in a Randomized Dose-Escalation Phase I Study with the Bacterial Enzyme IdeS—A Novel Therapeutic Opportunity”, PLoS One. 2015; 10(7):e0132011, published online Jul. 15, 2015, which are each incorporated herein by reference in their entireties). The agent may a B-cell-attenuating agent that destroys B-cells or otherwise reduces B-cells' production of IgG, such as bortezomib or rituximab.

The agent may be an agent that inhibits IgG opsonization of myelin debris, thereby inhibiting myelin uptake by vascular endothelial cells. For example, the agent may bind to the Fc receptor (FcRn receptor) on vascular endothelial cells in the subject, or interfere with interactions between the IgG and the Fc receptor, to inhibit myelin uptake by ECs. Fc receptors have been described in the literature (see, for example, Kuijpers T, “Fc-dependent mechanisms of action: roles of FcγR and FcRn receptors”, Clinical and Experimental Immunology, 178:89-91; and Pzik M et al., “FcRn: The architect behind the immune and non-immune functions of IgG and albumin”, J Immunol, 2015, May 15, 194(10):4595-4603, which are incorporated herein by reference in their entireties).

In some embodiments, the agent is monoclonal or polyclonal antibody, or antigen-binding fragment thereof, that binds to the Fc receptor (e.g., FcRn receptor) on vascular endothelial cells.

Agents that block FcRn have been reported in the literature and may be used in the invention:
1. ABDEGs (antibodies that enhance IgG degradation) are IgG molecules, where the Fc-part has been engineered to bind with high affinity to FcRn at both physiological and endosomal pH. These molecules can decrease the overall serum level of IgG in mice and have also been shown to ameliorate disease in an antibody dependent murine model of multiple sclerosis (Challa, D. K. et al. Autoantibody depletion ameliorates disease in murine experimental autoimmune encephalomyelitis. MAbs 5, 655-659, 2013.)
2. FcRn mAb, 1G3. Treatment with 1G3 significantly reduced the severity of the disease symptoms as well as the levels of total IgG and anti-AChR IgG relative to untreated animals. These data suggest that FcRn blockade may be an effective way to treat Ab-mediated autoimmune diseases. (Liu, L. et al. Amelioration of experimental autoimmune myasthenia gravis in rats by neonatal FcR blockade. J. Immunol. 178, 5390-5398, 2007.
3. A 26-amino acid peptide (SYN1436) binding to FcRn was developed, and was shown to decrease the overall serum-level of IgG upon injection into non-human primates. (Mezo, A. R. et al. Reduction of IgG in nonhuman primates by a peptide antagonist of the neonatal Fc receptor FcRn. Proc. Natl. Acad. Sci. USA 105, 2337-42, 2008.
4. An FcRn-binding affibody molecule (ZFcRn, affinity protein domains, 58 amino acids long) (Seijsing, J. et al. An engineered affibody molecule with pH-dependent binding to FcRn mediates extended circulatory half-life of a fusion protein. Proc. Natl. Acad. Sci. USA 111, 17110-5, 2014; Seij sing at al. In vivo depletion of serum IgG by an affibody molecule binding the neonatal Fc receptor. Scientific Reports. volume 8, 5141, 2018
5. M281: A Therapeutic Anti-FcRn Blocking Antibody for Rapid Clearance of IgG and IgG Autoantibodies in Immune Cytopenias and Other Auto/Allo-Immune Disease. Leona E Ling et al. Blood 2015 126:3472;
6. Rozanolixizumab (UCB7665; CA170_01519.g57 IgG4P) is an anti-human FcRn monoclonal antibody.

Other agents that act on Fc receptors are described in U.S. Patent Application Publication Nos. 2018/0305454 (Sexton Daniel J et al.), 2018/0291101 (Blumberg L J et al.), 2018/0127498 (Bhatta Petal.), 2018/0179258 (Ulrichts Petal.), 2018/0016334 (Kehry M et al.), 2017/0210801 (Kim S W et al.), and 2017/0209529 (Blumberg R S et al.), which are each incorporated by reference herein in their entireties.

One or a combination of two or more agents that inhibit vascular endothelial phagocytosis may be utilized. The agents in a combination may be the same class of agent (e.g., small molecule, nucleic acid, protein, antibody or antibody fragment) or different classes of agent, and may operate by the same mechanism of action in inhibiting vascular endothelial cell phagocytosis or by different mechanisms of action.

The clearance of damaged myelin sheaths is important to ensure functional recovery from central nervous system disorders associated with demyelination, such as spinal cord injury (SCI) and multiple sclerosis (MS). However, the cellular and molecular mechanisms for myelin clearance remain unclear and how myelin debris contributes to progression of demyelination disorders is poorly understood. The inventor shows herein that a previously unidentified role for microvessels and their lining endothelial cells (ECs) in engulfing myelin debris in two animal models of demyelination diseases: SCI and experimental autoimmune encephalomyelitis (EAE), a pre-clinical animal model of MS neural injury. The inventor demonstrates that IgG opsonization of myelin debris is required for its effective engulfment by ECs and that the autophagy-lysosome pathway is crucial for the degradation of engulfed myelin debris. The inventor further shows that following myelin uptake and autophagy-dependent processing, ECs exert critical functions beyond myelin clearance to promote progression of demyelination disorders by regulating inflammation, angiogenesis, and fibrosis. Specifically, ECs acquire the ability to facilitate the recruitment of bone marrow-derived macrophages to promote chronic inflammation and stimulate excessive proliferation for pathologic angiogenesis in both SCI and EAE models. Unexpectedly, myelin debris engulfment induces the endothelial-to-mesenchymal transition, a process that confers upon EC the ability to stimulate the endothelial-derived production of fibrotic components including collagen and fibronectin, suggesting a previously unknown function of ECs in fibrotic scar formation. Taken together, this demonstrates that microvascular ECs provide a novel route for the engulfment and processing of myelin debris through the autophagy-lysosome pathway, which in turn induces chronic pathology by promoting inflammation, angiogenesis, and fibrotic scar formation.

The efficient removal of degenerating myelin is important for functional recovery and inflammation resolution in the injured spinal cord. Therefore, understanding the cellular and molecular mechanisms for the clearance of myelin debris is an important therapeutic target. While professional phagocytes such as BMDMΦ are the major players in the clearance of myelin debris generated after SCI, the in vitro and in vivo experiments described herein demonstrated that microvascular ECs are the additional source for the clearance of myelin debris through the autophagy pathway. Importantly, the inventor revealed the biological significance of myelin debris uptake by microvascular ECs during SCI progression through a sequential regulation of inflammation, angiogenesis, and fibrotic scar formation.

Most of the knowledge of myelin debris phagocytosis comes from studies on macrophages and microglia. Receptors such as CR3, Mac-2 and LRP-1 are involved in myelin debris phagocytosis by macrophages/microglia [21]. It has been shown that ECs do not employ these receptors for myelin debris uptake. The “naked” myelin debris is not recognized by ECs and only IgG-opsonized myelin debris can be engulfed effectively, suggesting IgG receptors (FcγRs) may be involved in myelin debris engulfment by ECs. The family of FcγRs is highly expressed in macrophages to regulate a multitude of immune responses by interaction with IgG, immune complexes and opsonized particles or cells [34]. It is likely that ECs only express a small amount of FcγRs that engage in the engulfment of IgG-opsonized myelin debris, and this may thus account for the limited phagocytic capacity of ECs compared to strong phagocytosis of myelin debris by BMDMΦ. Compromised BBB leads to leakage of IgG in the injured area [35], which may be the source of IgG for oposinization.

Given the fact that brain ECs and other antigen presenting cells are able to engulf myelin debris and present myelin antigens to lymphocytes [36], it is possible that the specific antibodies may further opsonize myelin debris and facilitate its engulfment. Endogenous antibodies have been shown to promote the rapid clearance of myelin debris in mouse [37], but it is unknown which cell type(s) benefit from the opsonization by antibody for myelin debris clearance. Because IgG opsonization is required for myelin debris uptake by ECs, but not BMDMΦ (FIG. 20B), the inventor proposes that ECs, rather than BMDMΦ, are the major cell type that relies on antibody opsonization of myelin debris for in vivo myelin debris clearance.

Autophagy is a fundamental degradative pathway for degradation of intracellular proteins and organelles. One feature of autophagy is the formation of autophagosomes, which engulf cargoes and upon fusion with lysosomes form autolysosomes leading to the degradation of the enclosed materials [28]. Autophagy has recently emerged as an alternative mechanism for myelin debris clearance in Schwann cells [38,39]. The inventor shows that autolysosomes are involved in myelin debris degradation in microvascular ECs. It is to be determined whether autophagic processing of myelin debris is required for ECs' activities including ECs proliferation, inflammation and transition to fibroblast-like cells, which could be determined using an autophagy-deficient mouse model, for example, endothelium-specific atg5 or atg7 knockout mouse model [40].

The major form of vascular change in the injury area is angiogenesis during chronic stages of SCI. Surprisingly, it was found that the newly formed microvessels are structurally abnormal. They are dilated and appear more disorganized and tortuous. The mechanisms and biological outcomes for these vascular abnormalities are poorly understood after SCI. The inventor demonstrated that myelin debris could be one of the lesion-related factors that causes excessive EC proliferation, which may contribute to microvessel dilation at injury sites. Interestingly, the dilated microvessels in the injured spinal cords recapitulated the microvessels in mice lacking pericytes in an early stage of SCI [41], a cellular constituent in the neurovascular unit that has been recently reported to constrict microvessels [42]. Therefore, an alternative explanation for the microvessels' dilation could be that these newly formed microvessels have defects in pericytes maturation or/and coverage, which thus fail to constrict microvessels and lead to microvessels dilation. One of the most important features of neuroinflammation is the leukocyte recruitment from the blood circulation into the CNS, which requires the activation of ECs through an increased expression of adhesion molecules and secretion of cytokines/chemokines in ECs [43].

The study herein demonstrates that myelin debris engulfment activates microvascular ECs by increasing expression of adhesion molecules such as VCAM-1 and a variety of cytokines/chemokines, which could facilitate BMDMΦ recruitment to injury site. The RNA sequencing data suggests myelin debris might also promote BMDMΦ influx to injury site by increasing microvascular permeability to leukocytes, as indicated by the downregulation of genes related to cell junctions in myelin-ECs.

The fibrotic scars in the central region of injury sites, characterized by the excessive accumulation of pro-fibrotic proteins such as collagen and fibronectin, have been known to inhibit axon regeneration [44]. Fibroblasts, which are prominent in the injured epicenter, are thought of as the major contributor of the fibrotic scar formation by stimulating the production of collagen [30] and fibronectin [31]. However, little is known about the cellular origin of fibroblasts in the contusive injured spinal cords, whose dura is generally left intact and do not permit the invasion of meningeal fibroblasts into lesions [30,31,45]. Soderblom et al reported a resident source of perivascular fibroblasts from large blood vessels that migrate to the injury site to promote the fibrotic scar formation in mice after contusive SCI [46]. Besides resident fibroblasts, the activated fibroblasts or myofibroblasts may arise from different sources including resident fibroblasts, perivascular pericytes, bone marrow-derived precursors and others [47]. It has been recently reported that ECs have greater plasticity than previously appreciated, and could acquire fibroblast-like properties by undergoing EndoMT [33].

The study herein demonstrated that myelin debris induces EndoMT and confers microvascular ECs with fibroblasts-like properties including the production of endothelial-derived fibrotic proteins, suggesting microvascular ECs are additional source of fibroblasts or fibroblasts-like cells at the SCI lesion core. It takes a few days for myelin debris to significantly increase expression of fibronectin, collagen and a-SMA in microvascular ECs (FIGS. 7A, 7B, 7C, 7D, 7E, 7F, 7G, 7G-1, 7H, and 7I), coinciding with the delayed accumulation of perivascular fibroblasts at the injury core [46]. The inventor showed that EndoMT occurs during SCI, perhaps also in other demyelinating diseases, which could be confirmed using endothelial lineage-tracking system. The inventor further investigated how myelin debris induces EndoMT and showed myelin debris upregulates TGF-β1 expression. TGF signaling has been known as a master regulator of EndoMT [33] and participates in the formation of fibrotic scars in the injury site [48,49]. The TGF-β signaling is activated in several cell types within SCI lesion, including macrophages, astrocytes as well as ECs in blood vessels [44,50]. Thus, the inventor proposes that TGF signaling-mediated EndoMT in ECs may underlie the effects of TGF signaling on fibrotic scar formation in SCI lesions. It would be useful to determine the requirement of TGF signaling for myelin debris-induced EndoMT and other molecular mechanisms that govern myelin-induced EndoMT in the future studies.

The inventor has determined that microvessels and the lining microvascular ECs are a novel source for the clearance of myelin debris generated following SCI. Mechanistically, the inventor determined the requirement of IgG opsonization of myelin debris for efficient clearance by microvascular ECs. The engulfed myelin debris is then delivered through autophagy-lysosome pathway for intracellular degradation. Functionally, myelin debris engulfment by microvascular ECs contributes to three critical processes that are closely associated with the SCI progression, including robust angiogenesis that results in excessive and abnormal microvessels in SCI lesions, chronic inflammation, and endothelial-mediated fibrosis probably through EndoMT (FIGS. 8A, 8B, 8C, 8C-1, 8D, 8E, 8F, 8G, and 8H). It may be possible to reverse the effects of myelin-ECs at various stages of SCI by targeting these particular processes (myelin debris uptake, EndoMT, fibronectin/collagen deposition).

The autophagy-lysosome pathway (ALP) is described in Martini-Stoica H. et al., “The Autophagy-Lysosomal Pathway in Neurodegeneration: A TFEB Perspective”, Cell Death Dis., 2018 September; 9(9):858; Epub Aug. 28, 2018, which is incorporated herein by reference. Autophagy can potentially be suppressed at any stage of autophagic flux. Although autophagy and autophagy-related processes are dynamic, they can be broken down into several steps: (1) induction, (2) autophagosome formation, (3) autophagolysosome formation, and (4) delivery and degradation of the autophagic body. The primary step in inducing autophagy involves membrane nucleation, controlled by ULK complex and Beclin1. Inhibitors of positive regulators of of the ULK complex and Beclin1 have been demonstrated to block autophagy.

These include inhibitors to the MAP kinases, JNK1, ERK and p38. The induction of Atg protein and LC3 proteins is required for vesicle expansion and formation. Inhibitors of the class III PI3 kinases can block autophagy. In a later step of the autophagic process, inhibitors that inhibit lysosome acidification essentially block the formation of autophagosome and autophagic degradation. Further examples of various types of ALP inhibitors are described in Pasquier B et al., “Autophagy Inhibitors”, Cell Mol Life Sci., 2016 March, Epub Dec. 11, 2015, 73(5):985-1001; and Ha J et al., “Novel pharmacological modulators of autophagy: an updated patent review (2012-2015”, Expert Opin Ther Pat., 2016 November, Epub Aug. 8, 2016, 26(11):1273-1289, which are incorporated herein by reference in their entireties.

The methods of the invention may be used to reduce demyelination or an existing demyelinating condition, or treatment of an existing demyelination or a demyelinating condition, or may be used prophylactically to prevent demyelination or a demyelinating condition, or a recurrence thereof, in a subject. As used herein, in this context, the term “prevent” or “prevention” is inclusive of delaying the onset of demyelination or a demyelination condition and/or one or more symptoms of a demyelinating condition, and precluding the occurrence or reoccurrence of a demyelinating condition and/or one or more symptoms of a demyelinating condition. Thus, in some embodiments, the subject has demyelination or a demyelinating condition at the time the agent is administered, and the treatment or agent is administered as therapy.

The methods of the invention may also be used to reduce existing fibrosis, or treatment of an existing fibrotic condition, or may be used prophylactically to prevent a fibrosis or to prevent a fibrotic condition, or a recurrence thereof, in a subject. As used herein, in this context, the term “prevent” or “prevention” is inclusive of delaying the onset of fibrosis and/or one or more symptoms of a fibrotic condition, and precluding the occurrence or reoccurrence of fibrosis and/or one or more symptoms of a fibrotic condition. Thus, in some embodiments, the subject has the fibrosis or fibrotic condition at the time the treatment or agent is administered, and the treatment or agent is administered as therapy. Preferably, the agent is administered prior to fibrosis or the existence of a fibrotic condition.

Optionally, the methods of the invention further comprise, prior to administering the agent that inhibits vascular endothelial cell phagocytosis to the subject, identifying the subject as having the demyelinating condition, or testing for the presence of a demyelinating condition. Demyelination and a demyelinating condition in a subject can be identified using diagnostic methods known in the art, such as imaging (e.g., magnetic resonance imaging), changes in cerebral spinal fluid, and biopsies (see, for example, Kuhlmann T et al., “Diagnosis of inflammatory demyelination in biopsy specimens: a practical approach”, Acta Neuropathol, 2008 March, 115(3):275-287, which is incorporated herein by reference in its entirety).

Agents that inhibit vascular endothelial cell phagocytosis (also referred to herein as the active ingredients and compounds of the invention) are administered by any route appropriate to the location of the demyelination or potential demyelination to be addressed. Suitable routes of administration are described, for example in Remington: The Science and Practice of Pharmacy, University of the Sciences in Philadelphia (2005). For example, the agents may be administered intravascularly (e.g., intravenously), topically, orally, intramuscularly, intradermally, by inhalation or subcutaneously.

Suitable routes include oral, rectal, nasal, topical (including buccal and sublingual), vaginal and parenteral (including subcutaneous, intramuscular, intravenous, intradermal, intrathecal and epidural), inhalation, and the like. It will be appreciated that the preferred route may vary with for example the condition of the subject. In some embodiments, the agent is administered orally, nasally, rectally, parenterally, subcutaneously, intramuscularly, intravascularly (e.g., intravenously), intrathecally, intracerebroventricularly, or locally at a desired anatomical site, such as a site of existing demyelination or potential site of potential demyelination.

In some embodiments, the agent is administered directly into the subject's cerebrospinal fluid (CSF), e.g., by infusion, pump, or direct injection. For example, the agent may be administered intrathecally, by introduction into the spinal canal or into the subarachnoid space so that it reaches the CSF.

The fibrotic condition may be systemic. In some embodiments, the fibrotic condition is systemic sclerosis (e.g., limited systemic sclerosis, diffuse systemic sclerosis, or systemic sclerosis sine scleroderma), nephrogenic systemic fibrosis, cystic fibrosis, chronic graft vs. host.

In some embodiments, the fibrotic condition is scleroderma. In some embodiments, the scleroderma is localized, e.g., morphea or linear scleroderma. In some embodiments, the condition is a systemic sclerosis, e.g., limited systemic sclerosis, diffuse systemic sclerosis, or systemic sclerosis sine scleroderma.

In some embodiments, the fibrotic condition is a fibrotic condition of the lung, a fibrotic condition of the liver, a fibrotic condition of the heart or vasculature, a fibrotic condition of the kidney, a fibrotic condition of the skin, a fibrotic condition of the gastrointestinal tract, a fibrotic condition of the bone marrow or a hematopoietic tissue, a fibrotic condition of the nervous system, a fibrotic condition of the eye, or a combination of two or more of the foregoing.

The fibrotic condition may involve one or more of: the deposition of excess collagen, fibronectin and or other extracellular matrix components. In some embodiments, the fibrotic condition is lung fibrosis, glaucoma, scleroderma, liver fibrosis, cardiac fibrosis, renal fibrosis or renal failure.

The method of the invention may be carried out before, during, and/or after a surgical procedure. In some embodiments, the surgical procedure involves eye surgery, is performed on internal organs and tissues, is performed on the epidermal layer or is a procedure is to repair tendon injury. In some embodiments, the surgical procedure is performed on the epidermal layer and may lead to keloid formation or is associated with cosmetic or plastic surgery.

In some embodiments, the treatment or agent is administered to prevent or delay onset of post-operative surgical adhesions between abutting surfaces. In such cases, the method may comprise the steps of providing a sterile biomaterial and positioning the biomaterial between the abutting surfaces in the course of surgery. In some embodiments, the biomaterial is selected from the group consisting of serous and fibro-serous membranes, such as pericardium (e.g., bovine pericardium), peritoneum, fascia lata, dura mater, dermis, and small intestinal submucosa.

In some embodiments, the biomaterial is provided in the form of flat or textured sheets or strips.

In some embodiments, the surgery is selected from consisting of peritoneal, pericardial, obstetric, gynecological, neurosurgical, arthroscopic, orthopedic, plastic, reconstructive, muscle, or tendon surgery.

Optionally, the method further includes the step of suturing or stapling the biomaterial into place between the surfaces, or otherwise allowing the biomaterial to adhere into place between the surfaces.

The abutting surfaces may be two surface portions of the same tissue, surfaces from each of two or more discrete tissues, or the surfaces of a tissue and implanted material, for example.

The method of the invention can improve the healing of wounds or alleviation of demyelinating conditions and fibrotic disorders, resulting in reduced or improved scarring, comprising the use of agents that inhibit vascular endothelial cell phagocytosis. By “wounds or fibrotic disorder” is meant any condition which may result in the formation of scar or fibrotic tissue.

Treatment is effected by administering an effective amount of an agent to an subject in need of treatment, using a suitable route of administration, such as, but not limited to intravenous administration, oral administration, local administration, and inhalation. The agents, prodrugs or salts to be administered will typically be in the form of a pharmaceutical composition, optionally including pharmaceutically acceptable excipients and/or carriers.

The method of the invention may be used either immediately before or immediately after a surgical procedure to promote reduced or improved scarring.

For a lung fibrosis condition, potential routes of administration include orally and locally by inhalation. Inhaled agents can be formulated for use with dry powdered inhalers, metered dose inhalers or as a solution for nebulization. Oral agents can be formulated as a capsule, tablet or solution.

To treat subjects suffering from cardiac scarring, potential routes of administration include oral, intravenous, subcutaneous and as a coated stent to be implanted by a surgeon.

For subject undergoing eye surgery, or who have suffered wounds to the eye, or who have glaucoma, potential routes of administration include oral, intravenous, by injection directly into the affected region of the eye, as eye drops, by soaking into a sponge and being applied to the wound at the time of surgery, and contained within in an implant which may be surgically introduced.

For subjects undergoing spinal and back surgery, potential routes of administrations include oral, intravenous, and direct application to the wound during surgery, e.g., as a powder, solution or soaked into a sponge.

For subjects suffering from scleroderma and kidney fibrosis, some potential routes of administration include oral, intravenous, and coated implants.

For subjects undergoing plastic surgery, or who are prone to keloid formation, or who have skin burns, potential routes of administration include local application in the form of an ointment or a cream, or injected directly to the site of the wound, or orally or intravenously.

For subjects suffering from injury to tendons, including tendons in the hands, shoulders, elbows, hips, knees and feet, Dupuytren's disease, frozen shoulder (adhesive capsulitis), some potential routes of administration include local injection, as an ointment or cream, or administered directly to the site of the wound, oral or intravenous.

In some embodiments, the administration of the agent for any demyelinating condition is by injection into or adjacent a site of injury, by infusion, for example via an epidural intrathecal catheter, or by direct application to a site of injury or adjacent thereto.

In some embodiments, the method of the invention is used to control adhesions following a following a surgical procedure, comprising the steps of: providing an agent that inhibits vascular endothelial cell phagocytosis; and administration involves introducing the agent onto or into an area of the body following the procedure to inhibit adhesions, or scar formation. In some embodiments, the agent is used to prevent the formation of scar tissue following spinal surgery. In some embodiments, the agent is placed over the dura lining the spinal nerves and spinal cord. In some embodiments, the agent is placed around the great vessels after an anterior approach to the spine.

In some embodiments, the agent is used before, during, and/or after abdominal surgery to inhibit adhesions following abdominal surgery.

In some embodiments, the method is used in conjunction with spinal fusion and bone ingrowth for artificial disc replacement.

In some embodiments, the agent is incorporated into a hydrogel to effectuate slow release in the subject.

In some embodiments, the agent is released from a cardiac stent or other implant.

In some embodiments, one or more additional substances are administered such as agents that inactivate IgG in the subject (e.g., immunomodulating enzyme that hydrolyzes IgG in the subject, such as EndoS or IdeS).

An effective amount of an agent can be administered by one or multiple doses at one day or several days.

Agents that inhibit vascular endothelial cell phagocytosis can be administered to the body via a device such as a graft or other implant. In some embodiments, an agent that inhibits vascular endothelial cell phagocytosis is admixed with a matrix. Such a matrix can be a polymeric matrix, and can serve to bond the agent to a device. Polymeric matrices suitable for such use, include, for example, lactone-based polyesters or copolyesters such as polylactide, polycaprolactonglycolide, polyorthoesters, polyanhydrides, polyaminoacids, polysaccharides, polyphosphazenes, poly(ether-ester) copolymers (e.g. PEO-PLLA); polydimethylsiloxane, poly(ethylene-vinylacetate), acrylate-based polymers or copolymers (e.g. polyhydroxyethyl methylmethacrylate, polyvinyl pyrrolidinone), fluorinated polymers such as polytetrafluoroethylene and cellulose esters. Suitable matrices can be non-degrading or can degrade with time, releasing the agent(s), and optionally other compounds. An agent can be applied to the surface of a device by various methods such as dip/spin coating, spray coating, dip-coating, and/or brush-coating. An agent can be applied in a solvent and the solvent can be allowed to evaporate, thus forming a layer of agent onto the device. Alternatively, an agent can be located in the body of the device, for example in microchannels or micropores.

The method of the invention may be used in conjunction with other methods known in the art for treating demyelinating conditions, promoting the healing of wounds, and reducing scarring or treating fibrotic disorders. Numerous compounds have shown activity in the bleomycin animal model of pulmonary fibrosis, for example, and many of these can work additively or synergistically with agents and treatments used in the present invention to lead to a better therapeutic outcome; these are described in Moeller A., et al. The International Journal of Biochemistry and Cell Biology, 2008, 40:362-382. Specific examples are: N-acetyl cysteine, aminoguanidine, anti-VEGF antibody, Batimastat, Bosentan, dexamethasone, difluoromethylornithine, Etanercept, Gefitinib, Imatinib, methylprednisolone, Pentoxifylline, Pirfenidone, prednisolone, Rosiglitazone, TGF-beta antibody, TNF-alpha antibody, and Vinblastine.

Another aspect of the invention concerns a packaged dosage formulation for treating a demyelinating condition, comprising an agent that inhibits vascular endothelial cell phagocytosis in a pharmaceutically acceptable dosage in one or more packages, packets, or containers.

Another aspect of the invention concerns a kit for treating a demyelinating condition, comprising, in one or more containers, at least one agent that inhibits vascular endothelial cell phagocytosis. In some embodiments, the kit comprises a combination of two or more agents that inhibit vascular endothelial cell phagocytosis. In some embodiments, the kit further comprises an additional agent effective for inhibiting vascular endothelial cell phagocytosis. In some embodiments, the kit further comprises an additional agent effective for the treatment of one or more symptoms of a demyelinating condition.

Chemical reactions, reactants, and reagents that may be utilized to enhance solubility and make prodrugs of compounds are described in March's Advanced Organic Chemistry, 7^{th} edition, 2013, Michael B. Smith, which is incorporated herein by reference in its entirety.

Agents that inhibit vascular endothelial cell phagocytosis, and compositions comprising them, useful in the methods of the subject invention can be formulated according to known methods for preparing pharmaceutically useful compositions. Formulations are described in detail in a number of sources which are well known and readily available to those skilled in the art. For example, Remington's Pharmaceutical Science by E. W. Martin describes formulations which can be used in connection with the subject invention. In general, the compositions of the subject invention will be formulated such that an effective amount of at least agent that inhibits vascular endothelial cell phagocytosis is combined with a suitable carrier or diluent in order to facilitate effective administration of the composition.

The compositions used in the present methods can also be in a variety of forms. These include, for example, solid, semi-solid, and liquid dosage forms, such as tablets, pills, powders, liquid solutions or suspension, suppositories, injectable and infusible solutions, and sprays. The preferred form depends on the intended mode of administration and application. The compositions also preferably include conventional pharmaceutically acceptable carriers and diluents which are known to those skilled in the art. Examples of carriers or diluents for use with the active ingredients include, but are not limited to, water, saline, oils including mineral oil, ethanol, dimethyl sulfoxide, gelatin, cyclodextrans, magnesium stearate, dextrose, cellulose, sugars, calcium carbonate, glycerol, alumina, starch, and equivalent carriers and diluents, or mixtures of any of these. Formulations of the agents of the invention can also comprise suspension agents, protectants, lubricants, buffers, preservatives, and stabilizers. To provide for the administration of such dosages for the desired therapeutic treatment, pharmaceutical compositions of the invention will advantageously comprise between about 0.1% and 45%, and especially, 1 and 15% by weight of the total of one or more of the agents based on the weight of the total composition including carrier or diluent.

The agents that inhibit vascular endothelial cell phagocytosis can also be administered utilizing liposome technology, controlled release matrices, implantable pumps, and biodegradable containers. These delivery methods can, advantageously, provide a uniform dosage over an extended period of time.

The invention further provides kits, including at least one agent that inhibits vascular endothelial cell phagocytosis and pharmaceutical formulations, packaged into suitable packaging material, optionally in combination with instructions for using the kit components, e.g., instructions for performing a method of the invention. In one embodiment, a kit includes an amount of an agent that inhibits vascular endothelial cell phagocytosis, and instructions for administering an agent that inhibits vascular endothelial cell phagocytosis to a subject in need of treatment on a label or packaging insert. In further embodiments, a kit includes an article of manufacture, for delivering such an agent into a subject locally, regionally or systemically, for example.

As used herein, the term “packaging material” refers to a physical structure housing the components of the kit. The packaging material can maintain the components in a sterile state, and can be made of material commonly used for such purposes (e.g., paper, corrugated fiber, glass, plastic, foil, ampules, etc.). The label or packaging insert can include appropriate printed and/or digital instructions, for example, for practicing a method of the invention, e.g., reducing demyelination or treating a demyelinating condition, an assay for identifying a subject having a demyelinating condition, etc. Thus, in additional embodiments, a kit includes a label or packaging insert including instructions for practicing a method of the invention in solution, in vitro, in vivo, or ex vivo.

Instructions can therefore include instructions for practicing any of the methods of the invention described herein. For example, pharmaceutical compositions can be included in a container, pack, or dispenser together with instructions for administration to a subject to reduce demyelination or to treat a demyelinating condition. Instructions may additionally include indications of a satisfactory clinical endpoint or any adverse symptoms that may occur, storage information, expiration date, or any information required by regulatory agencies such as the Food and Drug Administration or European Medicines Agency for use in a human subject.

The instructions may be digital or on “printed matter,” e.g., on paper or cardboard within the kit, on a label affixed to the kit or packaging material, or attached to a vial or tube containing a component of the kit. Instructions may comprise voice or video tape and additionally be included on a computer readable medium, such as a disk (diskette or hard disk), optical CD such as CD- or DVD-ROM/RAM, magnetic tape, electrical storage media such as RAM and ROM and hybrids of these such as magnetic/optical storage media.

Kits can additionally include a buffering agent, a preservative, or an agent for stabilizing at least one compound of the invention. The kit can also include components for assaying for the presence of demyelination or a demyelinating condition, a control sample or a standard. Each component of the kit can be enclosed within an individual container or in a mixture and all of the various containers can be within single or multiple packages.

Kits can include packaging material that is compartmentalized to receive one or more containers such as vials, tubes, and the like, each of the container(s) including one of the separate elements to be used in a method described herein. Packaging materials for use in packaging pharmaceutical products include, by way of example only U.S. Pat. Nos. 5,323,907, 5,052,558 and 5,033,252. Examples of pharmaceutical packaging materials include, but are not limited to, blister packs, bottles, tubes, pumps, bags, vials, light-tight sealed containers, syringes, bottles, and any packaging material suitable for a selected formulation and intended mode of administration and treatment.

A kit may include one or more additional containers, each with one or more of various materials desirable from a commercial and user standpoint for use of the agents for reducing demyelination or for treating or preventing a demyelinating condition. Non-limiting examples of such materials include, but not limited to, buffers, diluents, carrier, package, container, vial and/or tube labels listing contents and/or instructions for use, and package inserts with instructions for use.

A label can be on or associated with a container containing an agent to be used in the method of the invention. A label can be on a container when letters, numbers or other characters forming the label are attached, molded or etched into the container itself; a label can be associated with a container when it is present within a receptacle or carrier that also holds the container, e.g., as a package insert. A label can be used to indicate that the contents are to be used for a specific therapeutic application. The label can also indicate directions for use of the contents, such as in the methods described herein.

In some embodiments of the kit, the agent(s) can be presented in a pack or dispenser device which can contain one or more unit dosage forms containing an agent disclosed herein. The pack can for example contain metal or plastic foil, such as a blister pack. The pack or dispenser device can be accompanied by instructions for administration. The pack or dispenser can also be accompanied with a notice associated with the container in form prescribed by a governmental agency regulating the manufacture, use, or sale of pharmaceuticals, which notice is reflective of approval by the agency of the form of the agent for human or veterinary administration. Such notice, for example, can be the labeling approved by the U.S. Food and Drug Administration for prescription drugs, or the approved product insert. Compositions containing an agent provided herein formulated in a compatible pharmaceutical carrier can also be prepared, placed in an appropriate container, and labeled for treatment of an indicated demyelination or demyelinating condition.

### Definitions

The terms “compounds of the invention” or “compounds of the present invention” (unless specifically identified otherwise), and grammatical variations thereof, refer to the compounds and classes of compounds disclosed herein, such as agents that inhibit vascular endothelial cell phagocytosis, including those specifically identified as well as a prodrug, metabolite, or derivative thereof, or a pharmaceutically acceptable salt of any of the disclosed agents, as well as all stereoisomers (including diastereoisomers and enantiomers), rotamers, tautomers and isotopically labeled compounds (including deuterium substitutions), as well as inherently formed moieties (e.g., polymorphs, solvates and/or hydrates). For purposes of this invention, solvates and hydrates are generally considered compositions.

The term “a,” “an,” “the” and similar terms used in the context of the present invention (especially in the context of the claims) are to be construed to cover both the singular and plural unless otherwise indicated herein or clearly contradicted by the context. Thus, for example, reference “a cell” or “an agent” should be construed to cover both a singular cell or singular agent and a plurality of cells and a plurality of agents unless indicated otherwise or clearly contradicted by the context.

The term “agent” refers to all materials that may be used as or in a pharmaceutical composition, or that may be a compound such as small synthetic or naturally derived organic compounds, nucleic acids, polypeptides, antibodies, fragments, isoforms, variants, or other materials that may be used independently for such purposes.

The term “small molecule” refers to a composition that has a molecular weight of less than about 3 kilodaltons (kDa), less than about 1 kDa, or less than about 1 kDa. Small molecules may be nucleic acids, peptides, polypeptides, peptidomimetics, carbohydrates, lipids, or other organic (carbon-containing) or inorganic molecules. A “small organic molecule” is an organic compound (or organic compound complexed with an inorganic compound (e.g., metal), that has a molecular weight of less than about 3 kDa, less than about 1.5 kDa, or less than about 1 kDa.

The term “isolated,” when used as a modifier of a composition of matter, such as a compound, means that the compositions are made by the hand of man or are separated from their naturally occurring in vivo environment. Generally, compositions so separated are substantially free of one or more materials with which they normally associate with in nature, for example, one or more protein, nucleic acid, lipid, carbohydrate, cell membrane. A “substantially pure” molecule can be combined with one or more other molecules. Thus, the term “substantially pure” does not exclude combinations of compositions. Substantial purity can be at least about 60% or more of the molecule by mass. Purity can also be about 70% or 80% or more, and can be greater, for example, 90% or more. Purity can be determined by any appropriate method, including, for example, UV spectroscopy, chromatography (e.g., HPLC, gas phase), gel electrophoresis (e.g., silver or coomassie staining) and sequence analysis (for nucleic acid and peptide). The compounds of the invention may be in isolated or substantially pure form.

The present invention includes derivatives of identified compounds, also referred to herein as pharmaceutically active derivatives. “Pharmaceutically active derivative” refers to any compound that upon administration to the subject or cell, is capable of providing directly or indirectly, the activity disclosed herein. The term “indirectly” also encompasses prodrugs which may be converted to the active form of the drug via endogenous enzymes or metabolism. The prodrug is a derivative of the agents according to the invention and presenting vascular endothelial cell phagocytosis inhibitory activity. The prodrug is converted into an agent according to the present invention by a reaction with an enzyme, gastric acid or the like under a physiological condition in the living body, e.g., by oxidation, reduction, hydrolysis or the like, each of which is carried out enzymatically. These compounds can be produced from compounds of the invention according to well-known methods. The term “indirectly” also encompasses metabolites of compounds according to the invention. Chemical reactions, reactants, and reagents useful for making derivatives can be found, for example, in March's Advanced Organic Chemistry, 7^{th} edition, 2013, Michael B. Smith, which is incorporated herein by reference in its entirety.

The term “metabolite” refers to all molecules derived from any of the compounds according to the invention in a cell or organism, preferably mammal. Pharmaceutically active metabolites of the compounds of the invention may be administered to a subject or contacted with a cell in vitro or in vivo.

The term “prodrug” refers to a chemical compound that can be converted by the body (i.e., biotransformed) to another chemical compound that has pharmacological activity. The prodrug may itself have pharmacological activity before conversion, or be inactive before conversion and activated upon conversion. Active prodrugs or inactive prodrugs of compounds of the invention may be administered to a subject or contacted with a cell in vitro or in vivo. Instead of administering a drug directly, a prodrug may be used instead to improve how a drug is absorbed, distributed, metabolized, and excreted (ADME). For example, a prodrug may be used to improve bioavailability when a drug itself is poorly absorbed from the gastrointestinal tract, or to improve how selectively the drug interacts with cells or processes that are not its intended target, which can reduce adverse or unintended effects of a drug.

Pharmaceutical formulations include “pharmaceutically acceptable” and “physiologically acceptable” carriers, diluents or excipients. As used herein the terms “pharmaceutically acceptable” and “physiologically acceptable” carriers, dilutents, or excipients include solvents (aqueous or non-aqueous), solutions, emulsions, dispersion media, coatings, isotonic and absorption promoting or delaying agents, compatible with pharmaceutical administration. Such formulations can be contained in a liquid; emulsion, suspension, syrup or elixir, or solid form; tablet (coated or uncoated), capsule (hard or soft), powder, granule, crystal, or microbead. Supplementary compounds (e.g., preservatives, antibacterial, antiviral and antifungal agents) can also be incorporated into the compositions. The phrase “pharmaceutically acceptable” indicates that the substance or composition must be compatible chemically and/or toxicologically, with the other ingredients comprising a formulation, and/or the mammal and/or cells being treated therewith. Examples of pharmaceutically acceptable carriers include but are not limited to saline, buffered saline, isotonic salinle, Ringer's solultion, dextrose, sterile water, deionized water, glycerol, ethanol, 5% dextrose in water, propylene glycol, and combinations of two or more of the foregoing.

The phrase “effective amount”, in the context of a subject, means an amount of at least one agent of the invention that (i) treats or prevents the particular disease, condition, or disorder (e.g., demyelinating condition or fibrotic condition) in a subject, (ii) attenuates, ameliorates, or eliminates one or more symptoms of the particular disease, condition, or disorder (e.g., demyelinating condition or fibrotic condition) in a subject, or (iii) prevents or delays the onset of one or more symptoms of the particular disease, condition, or disorder described herein (e.g., demyelinating condition or fibrotic condition) in a subject. It is not necessary that the symptoms be completely alleviated, prevented, or delayed, though they may be.

The phrase “effective amount”, in the context of a cell in vitro or in vivo, means an amount of at least one agent that (i) treats or prevents the particular disease, condition, or disorder (e.g., demyelinating condition or fibrotic condition) in a cell, (ii) attenuates, ameliorates, or eliminates one or more effects of the particular disease, condition, or disorder (e.g., demyelinating condition or fibrotic condition) in a cell, or (iii) prevents or delays the onset of one or more effects of the particular disease, condition, or disorder described herein (e.g., demyelinating condition or fibrotic condition) in a subject.

As used herein, a subject is “in need of” a treatment if such human or non-human animal subject would benefit biologically, medically or in quality of life from such treatment (preferably, a human). In some embodiments, the subject has a demyelinating condition and is in need of therapy. In other embodiments, the subject does not have a demyelinating condition and is in need of prophylaxis. In some embodiments, the subject in need of prophylaxis is at risk of developing demyelination or a demyelinating condition. In some embodiments, the subject is at increased risk of developing demyelination or a demyelinating condition relative to others in the population. In some embodiments, the subject is suspected to have demyelination or a demyelinating condition.

As used herein, the term “inhibit”, “inhibition” or “inhibiting” refers to the reduction or suppression of a given condition, symptom, or disorder, or disease (e.g., demyelination or demyelinating condition, or a fibrotic condition), or a significant decrease in the baseline activity of a biological activity or process (inhibits or suppresses vascular endothelial cell phagocytosis).

As used herein, the terms “subject”, “patient”, and “individual” refer to a human or non-human animal. Typically, the animal is a mammal. A subject also refers to for example, primates (e.g., humans), cows, sheep, goats, horses, dogs, cats, rabbits, rats, mice, fish, birds and the like. The animal may be an animal model of a disease, such as experimental autoimmune encephalomyelitis (EAE). In certain embodiments, the subject is a primate. In yet other embodiments, the subject is a human. The subject may be any age or gender.

As used herein, the term “treat”, “treating” or “treatment” of any disease or disorder refers in one embodiment, to ameliorating the disease or disorder (i.e., slowing or arresting or reducing the development of the disease or at least one of the clinical symptoms thereof). In another embodiment “treat”, “treating” or “treatment” refers to alleviating or ameliorating at least one physical parameter including those which may not be discernible by the subject. In yet another embodiment, “treat”, “treating” or “treatment” refers to modulating the disease or disorder, either physically, (e.g., stabilization of a discernible symptom), physiologically, (e.g., stabilization of a physical parameter), or both. In yet another embodiment, “treat”, “treating” or “treatment” refers to prophylaxis (preventing or delaying the onset or development or progression of the disease or disorder).

As used herein, the term “administration” is intended to include, but is not limited to, the following delivery methods: topical, oral, parenteral, subcutaneous, transdermal, transbuccal, intravascular (e.g., intravenous or intra-arterial), intramuscular, subcutaneous, intranasal, and intra-ocular administration. Administration can be local at a particular anatomical site, such as a site of demyelination, suspected demyelination, or anticipated demyelination, or systemic. In some embodiments, the agent is administered directly into the subject's cerebrospinal fluid (CSF), e.g., by infusion, pump, or direct injection. For example, the agent may be administered intrathecally, by introduction into the spinal canal or into the subarachnoid space so that it reaches the CSF. In some embodiments, the agent is administered locally to tissue at a site of demyelination, suspected demyelination, or anticipated demyelination by injection or topical application.

The agents to be used in the present invention can be formulated into pharmaceutically-acceptable salt forms. Pharmaceutically-acceptable salts of the compounds of the invention can be prepared using conventional techniques. “Pharmaceutically acceptable salt” includes both acid and base addition salts. A pharmaceutically acceptable salt of any one of the compounds described herein is intended to encompass any and all pharmaceutically suitable salt forms. Preferred pharmaceutically acceptable salts described herein are pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and. aromatic sulfonic acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S. M. et al., “Pharmaceutical Salts,” Journal of Pharmaceutical Science, 66:1-19 (1997), which is hereby incorporated by reference in its entirety). Acid addition salts of basic compounds may be prepared by contacting the free base forms with a sufficient amount of the desired acid to produce the salt according to methods and techniques with which a skilled person is familiar.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. Pharmaceutically acceptable base addition salts may be formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins and the like. See Berge et al., supra.

### Materials and Methods

Reagents. Chemical reagents were purchased from Sigma-Aldrich (St. Louis, Mo.) and cell culture media was purchased from Invitrogen (Carlsbad, Calif.), unless otherwise indicated. Carboxyfluorescein succinimidyl ester (CFSE; # C1157) was from Life Technologies (Carlsbad, Calif.). 3-Methuladenine (3-MA; BML-AP502) was from Enzo Life Sciences (Farmingdale, N.Y.). Rapamycin (#553210) was purchased from EMD Millipore (Burlington, Mass.). Recombinant mouse TGF-β1 (#5231) was from Cell Signaling Technology (Danvers, Mass.). Mouse MCP-1 ELISA kit (#432701) was from Bio Legend (San Diego, Calif.). Matrigel matrix growth factor reduced (#354230) was from BD Biosciences (San Jose, Calif.). Lysotracker red DND-99 (L7528; 1:5,000 for staining) was purchased from Invitrogen.

Antibodies. Anti-CD31 (#550274; 1:100 for IF) was from BD Biosciences (Franklin Lakes, N.J.). Anti-MBP (ab40390; 1:200 for IF; 1:1,000 for ELISA), anti-Ki-67 (ab15580; 1:200 for IF), anti-CD11b (ab133357; 1:100 for IF), anti-Von Willebrand Factor (vWF; ab11713; 1:100 for IF), anti-a-SMA (ab124964; 1:400 for IF), anti-fibronectin (ab23750; 1:200 for IF; 1:1,000 for WB), anti-collagen I (ab34710; 1:200 for IF), anti-GFAP (# ab53554, 1:400 and GAPDH (ab181602; 1:3,000 for WB) were purchased from Abcam (Cambridge, Mass.). The antibodies against LC3 (#4108; 1:100 for IF; WB 1:1,000), Atg5 (D5F5U, #12994, 1:1000 for WB), Rab5 (#3547; 1:50 for IF), Rab7 (#9367; 1:100 for IF) and GABARAP (#13733; 1:200 for IF) were purchased from Cell Signaling Technology (Danvers, Mass.). Another anti-MBP (MAB386; 1:1,000 for ELISA) was purchased from Millipore (Billerica, Mass.), anti-VCAM-1 (sc-8304; 1:250 for WB) was purchased from Santa Cruz Biotechnology (Dallas, Tex.) and anti-Lamp1 (1D4B; 1:25 for IF) was from Developmental Studies Hybridoma Bank. Anti-tubulin (DM1A; 1:5,000 for WB) was from Sigma-Aldrich (St. Louis, Mo.). Anti-p62 (PM045; 1:1,000 for WB) was from MBL (Woburn, Mass.). Anti-ubiquitin (FK2, # BML-PW8810-0500; 1:200 for IF) was from ENZO Life Sciences. Anti-Iba-1 (#019-19741, 1:200 for IF) was from FUJIFILM Wako Pure Chemical Corporation (Osaka, Japan). F4/80 and Mac-2 antibodies were produced by hybridoma cell lines (HB-198 for F4/80; TIB-166 for Mac-2) from American Tissue Culture Collection (ATCC, Manassas, Va.). VEGF neutralizing antibody (# AF-493-NA, 20 μg/ml for neutralization) and pan-TGF-β neutralizing antibody (# MAB1835R-100, 20 μg/ml for neutralization) were from R&D Systems (Minneapolis, Minn.). Alexa Fluor 488, 555, 647-conjuated secondary antibodies (1:600 for IF) and HRP-conjugated secondary antibody (1:3,000 for ELISA) were purchased from Invitrogen. IRDye-800CW or IRDye-680LT-conjugated secondary antibodies (1:20,000 for WB) were from LI-COR Bioscience.

Mice strains. C57BL/6J, C57BL/6-Tg (ACTB-EGFP)10sba and C3Fe.SWV-MBPshia mice were purchased from Jackson Laboratory (Bar Harbor, Me.) and maintained in pathogen-free animal facility in xxx. All animal protocols were approved by the Animal Care and Facilities Committee of xxx.

Spinal cord injury in mice. Thoracic spinal cord contusion injuries were performed on 8-10 week old C57BL/6J female mice. To expose the spinal cord, a laminectomy was performed on the T10 vertebrae. The contusion injury was induced using the NYU impactor with a 5 gram rod dropped 6.25 mm from the cord surface 1. Mice in the sham group were subjected to only a laminectomy without a contusion.

Active EAE induction in mice. EAE induction was performed as described previously with minor modifications 2, 3. All the animal experiments were approved by the Committee on the Use of Live Animals in Teaching and Research at xxx. 7-8 week old female C57BL/6J mice were used for EAE induction. The animals were housed in the Laboratory Animal Unit on a 12 hr day/night cycle, with food and water ad libitum, and were allowed to acclimatize for 1 week before disease induction.

Female mice were subcutaneously immunized with 200 μg MOG35-55 peptide (Genscript, Piscataway, N.J.) in complete Freund's adjuvant (3 mg/ml). Freshly prepared pertussis toxin (PHZ1174,ThermoFisher250 ng) in sterile PBS was injected intraperitoneally on day 0 and 48 h later.

EAE symptoms were scored daily as follows: 0, no clinical signs; 0.5, partially limp tail; 1, paralyzed tail; 1.5, hindlimb paresis or loss in coordinated movement; 2, loss in coordinated movement and hindlimb paresis; 3, one hindlimb paralyzed; 4, both hindlimbs paralyzed; 5, hindlimbs paralyzed, weakness in forelimbs; 6, moribund.

Generation of GFP+ bone marrow chimeras. GFP+ bone marrow chimeric mice were generated according to previous publication 4. Briefly, female C57BL/6 mice of 8-10 weeks of age were exposed to irradiation with 10 Gy X-ray and then intravenously injected with 5×106 bone marrow cells freshly collected from transgenic mice (C57BL/6-Tg(ACTB-EGFP)1Osb/J) constitutively expressing GFP. Efficient reconstitution was confirmed by postmortem examination of circulating blood for GFP+ cells. On average, 80% the transplant engraftment efficiency was achieved.

Cell cultures. Primary mouse brain microvascular endothelial cells (BMECs, # C57-6023; Cell Biologics, Chicago, Ill.) were cultured in complete mouse endothelial cell medium (Cell Biologics, Chicago, Ill.). The primary BMECs were confirmed by staining two endothelial-specific markers, CD31 and vWF. Primary BMECs cultures were used from passage 3 to 5 and they are all positive for both CD31 and vWF (data not shown). Mouse brain microvascular endothelial cell line (bEnd.3, CRL-2299) and a mouse neuroblastoma cell line, Neuro-2a (N2A, CCL-131) were purchased from ATCC. Endothelial cell line bEnd.3 and Neuro-2a cell line were cultured in DMEM containing 5% or 10% FBS and 1% penicillin/streptomycin.

Bone marrow-derived macrophages (BMDMΦ) from female C57BL/6J mice were prepared as previously described 4. Briefly, bone marrow cells from mice 6-8 weeks of age were collected from femoral shafts by flushing the marrow cavity of femurs of with DMEM supplemented with 5% NCS. Cells were cultured for 7 days in DMEM supplemented with 15% conditioned medium from L929 cells (a source of M-CSF) and 5% NCS. All of the cell types were cultured in an incubator at 37° C., with 5% CO₂.

Generation of Atg5 knockout EC cell line by CRISPR/Cas9 technique. Single guide RNA targeting sequence, atgaaggcacacccctgaaa (SEQ ID NO:24), was selected to target the third exon of mouse Atg5 gene. The expression of guide RNA & scaffold RNA is driven under U6 promoter. The U6 promotor and guide & scaffold cassette was incorporated into a Cas9 expressing backbone vector tagged with EGFP. The sequence of the U6 promotor, guide RNA & scaffold RNA was confirmed by the sequencing, then transfected into mouse endothelial cells line bEnd.3 using FuGENE® 6 (# E2693, Promega) followed by the FACS sorting process. The method for CRISPR-Cas9 has not been published and a full characterization of this method will be published elsewhere. Forty eight hours later, cells were trypsinized into single cells, and sorted according to the GFP+ signal into 96-well plate. After colony expansion, the protein expression of Atg5 was analyzed by western blotting. The genomic DNA was extracted from those clones which completely lost the Atg5 protein expression, then the targeted region was PCR amplified with the primer set flanked the targeted region. PCR amplicons was purified with the PCR purification kit (#28104, QIAGEN), T7E1 (# E3321, NEB) assay was carried out to confirm the mismatch occurred at very specific site.

Preparation and fluorescent labeling of myelin debris. Myelin debris was isolated as described previously 5. The endotoxin concentration of myelin debris was below the detection limit by Pierce™ LAL Chromogenic Endotoxin Quantitation Kit (Thermo Fisher). The concentration of myelin debris was 1 mg/ml for cell culture throughout this study.

Fluorescent labeling of myelin debris was performed using a non-cytotoxic dye, CFSE. The dye enters the myelin debris by passive diffusion, where it covalently couples to free amine groups. Myelin debris (1 mg/ml) was labeled with 50 μM CFSE and incubated protected from light at 37° C. for 15 min. The labeled myelin debris was washed with PBS containing 100 mM glycine at 14,000 rpm for 15 min, then washed with PBS twice at 14,000 rpm for 15 min each. The myelin debris pellets were collected and refilled to 100 μl with PBS. CFSE-labeled myelin debris co-localizes with anti-MBP signals (FIG. 13), indicating the reliability of either CFSE labeling or MBP staining for myelin debris uptake analysis. Moreover, CFSE-myelin debris isolated from Shiverer (Mbpshi/shi) mice lacking functional MBP was fluorescent green but are negative for MBP signals (FIG. 13), verifying the specificity of the MBP antibody used. As such, CFSE labeling was used as the primary method for myelin debris engulfment analysis.

Myelin debris uptake assay. 2×104 BMECs were seeded on coverslips in 24-well plates with medium containing 1% FBS. CFSE-labeled myelin debris was added to the BMECs cultures for indicated time periods at a final concentration of 1 mg/ml. Non-ingested myelin debris was washed away from cell surface with EDTA for 30 seconds, citric acid for 1 min. Myelin debris uptake was analyzed by standard assays including confocal fluorescent imaging, flow cytometry and ELISA detection of intracellular MBP as described below.

To study the roles of CR3 or Mac-2 in EC uptake of myelin debris, BMECs were pre-incubated with anti-CR3 or Mac-2 mAb (hybridoma supernatant) or control IgG for 3 hr. A combinated incubation of anti-CR3 and Mac-2 mAb was included to cover any compensatory effect. 1 mg/ml CFSE-labeled myelin debris was added and cultured for 72 hr. After removal of non-ingested myelin debris, BMECs on coverslips were analyzed for myelin uptake by fluorescent imaging. Meanwhile, BMDMΦ were included as positive control cells to test the utility of the used neutralizing antibodies.

Serum-dependent uptake of myelin debris was performed as described above, except the use of FBS concentrations, where 0%, 1% or 5% FBS-containing medium were used to culture BMECs for 48 hr. We determined that cell viability was indistinguishable among the different serum concentrations during the 48 hr culture period (data not shown).

To test the requirement of IgG or complement opsonization for myelin debris uptake by BMECs, FBS was heated for 20 min at 56° C. to inactivate complement, and at 70° C. to inactivate IgG. The complement-inactivated or IgG-inactivated FBS was then used in BMECs cultures for the analysis of myelin debris uptake as described above.

Media containing no serum or IgG-inactivated serum were supplemented with 100 μg/ml purified IgG to test whether IgG supplementation could rescue myelin debris uptake. CFSE-labeled myelin debris was then added in BMECs cultures for 72 hr, followed by MBP ELISA assay to detect engulfed myelin debris. To determine whether myelin debris pre-coated with IgG could bypass the requirement of IgG in medium for myelin debris uptake, IgG-opsonized myelin debris was added to BMECs culture medium with no serum or IgG-inactivated serum. IgG-opsonized myelin debris was first prepared by incubating myelin debris with 100 μg/ml purified IgG at room temperature for 30 min and then overnight at 4° C. Afterwards, it was washed 3 times of PBS to remove uncoated IgG.

EC uptake of necrotic neuronal cell bodies and zymosan. Neuronal cells were differentiated from Neuro-2a (N2A) cells, a mouse neuroblastoma cell line as previously described 6. Briefly, N2A cells were normally cultured in DMEM medium with 10% FBS. To induce differentiation of N2A cells into neuronal cells, N2A cells were cultured in DMEM with 2% FBS and 20 μM retinoic acid for 3 days, and medium was changed every 24 hr. Cells having one or more neurites of a length more than twice the diameter of the cell body were considered as differentiated neuronal cells. The N2A-differentiated neuronal cell bodies were generated by heating at 60° C. for 1 hr. The necrotic neuronal cell bodies were fluorescently labelled with CFSE and incubated with BMECs at 8:1 ratio for 72 hr, followed by immunostaining and confocal imaging. BMDMΦ uptake of necrotic cell bodies was performed as positive control at the same ratio for 6 hr. For zymosan uptake, commercially available zymosan was labeled with CFSE and incubated with BMECs or BMDMΦ for 72 hr and 3 hr, respectively.

Flow cytometry analysis of myelin debris uptake. BMECs were treated with or without CFSE-labeled myelin debris for 72 hr, and washed to remove non-ingested myelin debris. BMECs were collected and resuspended in PBS, followed by immediate detection with a BD FACS Canto flow cytometer (Becton Dickinson).

Enzyme-linked immunosorbent assay (ELISA) detection of engulfed myelin debris. To detect the engulfed myelin debris in ECs, we performed MBP-specific sandwich ELISA as previously described 7, 8, with rabbit MBP antibody being the capturing antibody and rat MBP antibody being the detecting antibody. Myelin-laden ECs were lysed in RIPA buffer with 1% protease inhibitor cocktail (ThermoFisher). Protein concentration was measured using BCA kit and 10 μg of total protein was loaded for ELISA detection of MBP. Rabbit MBP antibody in coating buffer (0.5 M sodium carbonate buffer, pH 9.6) was bound to NUNC MaxiSorp™ ELISA plates (ThermoFisher) overnight at 4° C., followed by blocking with 2% BSA for 2 hr at 37° C. and washed five times with PBS containing 0.05% Tween-20. Lysates were added to react with MBP detecting antibody overnight at 4° C. Rat MBP antibody was used to detect immobilized MBP, followed by incubation with HRP-conjugated antibody and detection at 450 nm after reaction with TMB colorimetric substrates (#421101; BioLegend). ELISA detection of cytokines in cell culture supernatant was performed following the instructions of manufacturer.

Myelin debris engulfment by microvessels-like structures. Primary BMECs were seeded on the polymerized Matrigel and cultured at 37° C. for 24 hr to form the tubular structures and then incubated with CFSE-myelin debris. After removal of non-ingested myelin debris, cells on Matrigel-coated coverslips were fixed with 2% paraformaldehyde (PFA), followed by regular immunostaining.

Images were acquired with Nikon A1 laser scanning confocal microscope (Nikon, Japan) and the slice view of the tubular structures from both x-y axis and x-z axis was collected from the Nikon Elements analysis software.

Histology and immunofluorescent staining. To collect the spinal cords from SCI mice, EAE mice, or normal mice with spinal cord injections, mice were anaesthetized and then rapidly perfused transcardially with 0.9% saline, followed by 4% PFA. Spinal cords were rapidly collected and fixed in 4% PFA overnight, and then cryoprotected in 30% sucrose overnight at 4° C. before being sectioned for immunohistochemistry. For immunofluorescent staining, the sectioned slices were blocked with PBS containing 0.3% Triton X-100 and 1% BSA for 1 hr at room temperature. After incubation with primary antibodies overnight at 4° C., the samples were washed, followed by incubation with secondary antibodies for 1.5 hr at room temperature. Cell samples were fixed with 4% PFA in PBS for 20 min, permeabilized with 0.2% Triton X-100 for 8 min and blocked with 5% BSA for 30 min, then incubated with primary antibodies overnight at 4° C. followed by secondary antibodies at room temperature for 1.5 hr.

Histology quantification. Quantifications of microvessel size were performed by unbiased researchers. To quantify the size of microvessels in three consecutive regions (injured, marginal and uninjured) of SCI, we first classified the three regions with GFAP staining as a major reference according to a previous publication with modification 9. Using Nikon NIS-Elements software, the total area of the spinal cord and the area of the GFAP+ regions were outlined and measured at 200 μm intervals over a 2 mm distance, centered on the lesion core. The injured regions were defined as the regions spanning with a radius of around 300 which were negative for GFAP but densely positive for nucleus staining (Hoechst staining). The marginal regions, within the GFAP glia scar, were considered as 300-600 μm away from the epicenter, and the uninjured regions were considered as regions>600 μm away from the epicenter. We usually analyzed the normal regions that were more than 1000 μm away from epicenter. For microvessel size quantification in mouse EAE spinal cord samples, we measured microvessel diameter in both non-demyelinated and demyelinated regions in T10 segment, where mild demyelination occurred. At least 20 microvessels with clear CD31 signals on each region were included for diameter analysis using image J Pro Plus 6.0 (Media Cybernetics, Rockville, Md.).

To quantify the microvessel uptake of MBP+ myelin debris in mouse SCI samples, we focused on uninjured and injured regions as classified above. The uninjured region represents the region without myelin debris and the injured region represents the region that accumulates myelin debris. X-Y, X-Z and Y-Z views were included to carefully assess the presence of myelin puncta within microvessels. Microvessels containing at least one fluorescently clear MBP+ puncta were considered as myelin-containing microvessels.

To quantify the microvessel uptake of MBP+ myelin debris in mouse EAE samples, we focused on T10 regions at early time points (pre-onset stage) when mild demyelination, but not severe demyelination, occurred. This choice allowed us to observe the microvessel uptake of myelin debris before myelin removal by other professional phagocytes such as microglial cells. Similar quantification of myelin-containing microvessels were performed as above.

To quantify the Ki67 positive microvessels in SCI samples, the microvessels positive for both Ki67 and CD31 staining were counted in the injured regions of 1-weeks post SCI or normal spinal cords.

For quantification of GFP+ BMDC infiltration across microvessels at different time points of SCI, we stained spinal cord tissues with CD31 in GFP+ bone marrow chimeric mice after SCI and counted the number of GFP+ BMDCs that are closely associated with microvessels in one whole field with an area of 0.044 mm2.

For quantification of Iba-1+ cells in EAE microvessels, we stained Iba-1 and CD31 in T10 segment of 7 days post EAE spinal cords and counted the number of Iba-1+ cells showing close association with per normal-sized or enlarged microvessels. 15-days post EAE samples had similar results with 7 days post EAE samples in Iba-1+ cells association with enlarged microvessels.

Oil Red O (ORO) staining. ORO staining was performed to detect intracellular neutral lipid accumulation in injured spinal cords and cultured BMECs. Spinal frozen sections or fixed cells were dehydrated in 100% propylene glycol for 5 min, then stained with 0.5% ORO solution at 60° C. for 8 min. The samples were then processed with 85% propylene glycol for 5 min followed by distilled water rinsing for 3 times. Stained samples were imaged with a confocal laser scanning microscope.

Lysotracker red staining and analysis. BMECs were treated with or without CF SE-myelin debris for 72 hr. After washing away the non-ingested myelin debris, cells were stained by Lysotracker red (1:5,000) and Hoechst (1:1,000) for 15 min at 37° C. After three rapid washes in PBS, cells were imaged immediately with a Nikon A1 laser scanning confocal microscope using a 60×/1.49NA oil immersion objective. The number and size of Lysotracker red positive puncta were analyzed using Image J Pro Plus 6.0. The size of Lysotracker red positive puncta was measured as the diameter of the puncta. More than 200 puncta in at least 20 cells from 3 independent experiments were measured.

Starvation and drug treatments for autophagy assays. BMECs cultured with 5% FBS containing medium were used as negative control for autophagy. The positive control for autophagy induction, BMECs, were starved with Hanks Balanced Salt Solution for 6 hr to induce autophagy. 10 μM chloroquine, an inhibitor of lysosomes, or 1 μM of rapamycin, an inducer of autophagy, was added to BMECs cultures for 24 hr after myelin debris treatment. 2 mM of 3-MA, a PI3K inhibitor of autophagy was cultured with ECs for 48 hr, starting from the last 24 hr treatment of myelin debris to the subsequent 24 hr of medium wash. Cells grown on coverslips were fixed and stained with Oil Red 0 for detection of myelin-derived lipids as above described.

Autophagy measurements and co-localization analysis. BMECs were immunostained with antibodies against endogenous LC3 or GABARAP. The number of LC3+ or GABARAP+ puncta per image was counted automatically with Image J after intensity thresholding of images.

The number of LC3+ puncta was counted from 3 independent experiments that include at least 50 cells every triplicate.

Co-localization between two channels of confocal images with maximum intensity projection was performed with Image Pro Plus. Pearson's correlation coefficient (R) was analyzed. Co-localization of Lysotracker red puncta and engulfed CFSE-myelin debris was analyzed on at least 10 cells from 3 independent experiments. The co-localization between myelin debris and autophagosomes or endosomes was performed in the same way. At least 10 cells from 3 independent experiments were included for the co-localization between myelin debris and autophagosomes by LC3 or GABARAP antibody staining, or co-localization between myelin and endosomes by Rab5 or Rab7 staining.

Propidium iodide (PI) staining assay for cell death analysis. Wild-type BMECs or Atg5 knockout BMECs were cultured with DMEM medium containing 5% FBS in 24-well plate. To test the toxicity of different autophagy-related drugs, BMECs were treated with or without myelin debris and were treated with or without 1 mM 3-MA (48 hr), 10 μM chloroquine (24 hr), or 1 μM rapamycin (24 hr). After reaching 70% confluence, cells were washed with 1×PBS and fixed with 4% PFA. The fixed cells were stained with propidium iodide and Hoechst, followed by imaging and quantification of PI positive nucleus using Nikon Element software.

Image acquisition. Samples from spinal cords and cell culture were imaged with a Nikon Ti-E microscope (Nikon Instruments, Melville, N.Y.) using 10×objective for large images acquisition with 25% overlapping. Regions of interest were imaged with Nikon A1 laser scanning confocal microscope using a 20×objective or a 60×/1.49NA oil immersion objective. All confocal images were acquired with a spacing of 0.25 μm or 0.5 μm between z-sections in Nikon NIS-Elements software and are maximum intensity projections of z stacks. In some images, volume view of xyz axis with or without 3D rendering, and slice view of x-y, x-z or y-z axis were applied. Gamma correction was applied in some images.

Quantitative RT-PCR. Total RNA from cells was extracted using TRIzol. cDNA was reverse transcribed from 1 μg of RNA using qScript Flex cDNA Synthesis Kit (#95047; Quanta Biosciences, Beverly, Mass.) according to the manufacturer's instructions. A total of 20 μL reaction system was prepared for quantitative RT-PCR using perfecta SYBR Green supermix (#95054; Quanta Biosciences). All reactions were run in triplicates using a real-time PCR system (CFX96; BioRad), and the specificity of every reaction was determined by a melting curve analysis. The expression level of target genes was normalized to GAPDH and calculated using the Δ Δ^{Ct} method. Primers used for the reactions are as follows:

GAPDH-forward:
(SEQ ID NO: 1)
5'-ATCAACGACCCCTTCATTGACC-3'
reverse:
(SEQ ID NO: 2)
5'-CCAGTAGACTCCACGACATACTCAGC-3'
Il-4-forward:
(SEQ ID NO: 3)
5'-TCAACCCCCAGCTAGTTGTC-3'
reverse:
(SEQ ID NO: 4)
5'-TGTTCTTCGTTGCTGTGAGG-3'
Il-6-forward:
(SEQ ID NO: 5)
5'-TGGGAAATCGTGGAAATGAG-3'
reverse:
(SEQ ID NO: 6)
5'-CTCTGAAGGACTCTGGCTTTG-3'
Mcpl-forward:
(SEQ ID NO: 7)
5'-ATGCAGGTCCCTGTCATGCTT-3'
reverse:
(SEQ ID NO: 8)
5'-CATTGGGATCATCTTGCTGGT-3'
vegf-forward:
(SEQ ID NO: 9)
5'-CAGGCTGCTGTAACGATGAA-3'
reverse:
(SEQ ID NO: 10)
5'-GCATTCACATCTGCTGTGCT-3'
iNOS-forward:
(SEQ ID NO: 11)
5'-TTGGAGCGAGTTGTGGATTGT-3'
reverse:
(SEQ ID NO: 12)
5'-GTAGGTGAGGGCTTGGCTGA-3'
Collagen 1 α1-forward:
(SEQ ID NO: 13)
5'-TGACTGGAAGAGCGGAGAGT-3'
reverse:
(SEQ ID NO: 14)
5'-AGACGGCTGAGTAGGGAACA-3'
Collagen 1 α2-forward:
(SEQ ID NO: 15)
5'-CACCCCAGCGAAGAACTCATA-3'
reverse:
(SEQ ID NO: 16)
5'-GCCACCATTGATAGTCTCTCC-3'
Collagen 5 α2-forward:
(SEQ ID NO: 17)
5'-TTGGAAACCTTCTCCATGTCAGA-3'
reverse:
(SEQ ID NO: 18)
5'-TCCCCAGTGGGTGTTATAGGA-3'
α-SMA-forward:
(SEQ ID NO: 19)
5'-GTCCCAGACATCAGGGAGTAA-3'
reverse:
(SEQ ID NO: 20)
5'-TCGGATACTTCAGCGTCAGGA-3'
Tgf-β1-forward:
(SEQ ID NO: 21)
5'-GGATACCAACTATTGCTTCAGCTCC-3'
reverse:
(SEQ ID NO: 22)
5'-AGGCTCCAAATATAGGGGCAGGGTC-3'

RNA-sequencing and data analysis. Mouse brain microvascular endothelial cells (bEnd.3) were plated at equal density in cell culture dishes and allowed to rest overnight prior to the addition of 1 mg/mL myelin debris prepared as previously described. Total RNA was isolated from each of 2 biological replicates for control and myelin debris treated cells for 72 hours using the TRIzol® Plus RNA Purification Kit (Thermo Fisher). Selection of mRNA from total RNA was accomplished using the NEBNext Poly(A) mRNA Magnetic Isolation Module (NEB # E7490). From the total mRNA obtained, cDNA was generated using the high fidelity ProtoScript II Reverse Transcriptase (NEB) with a random primer mix to generate fragments. The double-stranded cDNA was purified using 1.8× Agencourt AMPure XP Beads prior to end preparation for adaptor ligation. The ligation reaction was performed using AMPure XP Beads and enriched via PCR and followed by a final purification using the Agencourt AMPure XP Beads. Quality of the resulting library was examined using Agilent High Sensitivity DNA Bioanalyzer Chips (Agilent Technologies 5067-4626) and quantified by KAPA Library Quantification Kits for Illumina sequencing platforms (KAPA Biosystems KK4824). Single-end sequencing was performed on the Illumina HiSeq 2000 DNA Sequencer.

For mRNA-seq data analysis, the resulting sequences were trimmed of their Illumina indexing adaptors using Trimmomatic¹⁰. All reads between 50 and 100 bases were included in further analyses. Any reads with greater than 2 Ns were considered to be low quality, and thus discarded. Unique reads were aligned to the Mus musculus genome using TopHat2. A total of 4 mismatches between the reference genome and sample were allowed during alignment to account for strain differences between the endothelial cell line and the C57BL/6 reference genome. The TopHat2 mapped reads were further processed (filtered, sorted and indexed) with Samtools such that reads mapped to a single gene were used for further analysis^{11, 12}. The uniquely mapped reads were then used to generate counts for each annotated gene using HTSeq (from Bioconductor version 3.0.2)¹³. Finally, differential expression analysis of count tables for control versus myelin debris treated comparisons at each time point was performed in DESeq2 (1.8.1, Benjamini-Hochberg FDR correction)¹⁴⁻¹⁶.

Western blot. BMECs were lysed in RIPA lysis and extraction buffer (G-Biosciences, MO) containing 1% protease inhibitor cocktail. Protein concentration of the lysates was measured using BCA kit. 50 μg of total protein was resolved by either 4%-20% gradient SDS-PAGE gel or 7.5%-15% gel. SDS-PAGE gels were then transferred to PVDF membrane (Bio-rad) at 350 mA for 2 hr using a wet transfer system (Bio-rad). After blocking with 5% non-fat milk in TBST for 1 hr, the membrane was probed with primary antibodies diluted in TBST overnight at 4° C. After washing in TBST three times for 5 min each, the washed membrane was incubated with secondary antibodies conjugated with IRDye-800CW or IRDye-680LT for 1 hr at room temperature. The fluorescent signals of the probed bands were imaged with an Odyssey Infrared Imaging system (LI-COR Bioscience, Lincoln, Nebr.), followed by image processing in Adobe Photoshop CS4. Densitometric quantification of the bands was analyzed with Image J. All values were normalized to tubulin or GAPDH as indicated in figure legends.

Cells proliferation assay. Ki-67 labeling assay and cell number counting assay were used to assess the proliferation capacity of BMECs following myelin debris engulfment. Either 5×10³ cells or 2×10⁴ cells were seeded and cultured for 24 hr in 24-well plates for Ki-67 labeling or cell number counting, respectively, followed by treatment with or without 1 mg/ml myelin debris for 24-120 hr. For Ki-67 labeling, the fixed cells on coverslips were stained with anti-Ki-67 and Hoechst as marker of proliferation and nucleus. The Ki-67 and Hoechst signals were imaged using fluorescent microscope and counted with Nikon Elements software. The percentage of Ki-67⁺ and the total number of cells with Hoechst were calculated to show the proliferation capacity of endothelial cells. For cell counting assay, after the same treatment with Ki-67 assay, the cells in 24-well plates were trypsinized and counted using a hemocytometer.

To examine the role of autophagy in myelin-induced EC proliferation, myelin debris was added to wild-type or Atg5 knockout BMECs for 72 hr, followed by cell number counting.

To examine VEGF function in myelin-induced EC proliferation, VEGF neutralizing antibody (20 μg/ml) or control IgG was added to BMECs 3 hr prior to myelin debris upload for 72 hr. Cell number was counted.

To examine ECs proliferation after engulfment of necrotic neuronal cell bodies, BMECs were seeded in 24-well plate and necrotic cell bodies were added to BMECs at 8:1 ratio. After co-culture for 72 hr, BMECs were stained with Hoechst to label nucleus. Necrotic cell bodies were negative for Hoechst staining. The number ECs was quantified by counting the nucleus number and calculated per mm².

In vivo Matrigel plug angiogenesis assay. Matrigel plug angiogenesis assay was performed with a method modified from our previous publication¹⁷. Briefly, 8×10⁵ primary myelin-BMECs or naïve BMECs were mixed with 100 μl of pre-cooled Matrigel solution. The mixtures were subcutaneously injected in mice. Myelin-ECs, naïve-ECs and PBS (as blank control) were injected in the same mouse at different sites. After implantation for 7 days, the Matrigel plugs were excised, immediately photographed with a MVX10 Macro Zoom microscope (Olympus), subsequently followed by regular tissue histology and immunofluorescent staining for CD31 to label microvessels, whose density was analyzed and calculated as the percentage of CD31 positive area to the whole field.

BMDMΦ adhesion on endothelial cells. BDECs were seeded in 24-well plates and treated with or without 1 mg/ml CFSE-labeled myelin debris for 72 hr to induce myelin-ECs. After removal of the myelin debris remnant in the culture, 3×10⁵ BMDMΦ were added to naïve-ECs and myelin-ECs monolayer. After 1 hr adhesion, non-adhered BMDMΦ were gently washed away with PBS for 5 times. The adhered BMDMΦ on EC monolayer were stained with Mac-2 antibody and imaged by a combination of phase contrast and fluorescent microscopy for visualization of endothelial monolayer and Mac-2⁺ BMDMΦ, respectively. The number of Mac-2⁺ BMDMΦ that adhered on endothelial monolayer was counted and normalized to the number of ECs as the percentage of BMDMΦ adhesion onto endothelial monolayer.

To investigate whether any remaining myelin debris in BMECs could affect BMDMΦ adhesion, we treated BMECs with myelin debris in the presence of 70° C. heated serum (IgG-inactivated) for 72 hr. These BMECs, which were exposed to myelin debris but unable to engulf myelin debris, were used for the above macrophages adhesion assay.

To examine whether Atg5 knockout BMECs have any effects on macrophage adhesion, we performed the same assay using Atg5 knockout BMECs as above described.

BMDMΦ chemotaxis towards endothelial cells supernatant. A modified transwell assay was used to examine BMDMΦ chemotaxis towards EC's supernatant. After BMECs engulf myelin debris for three days, they were quickly washed with PBS three times. This step gets rid of most remaining non-engulfed myelin debris in ECs. After the washes, myelin-laden BMECs were cultured for 24 hr in fresh culture media, followed by collection of cell culture supernatant from the myelin-laden ECs. The cell culture supernatant was clarified by centrifugation to remove any remaining myelin debris. Then, the BMEC supernatant was placed in the bottom chamber of the transwell, and BMDMΦ were seeded on the upper chamber. After chemotaxis for 6 hr, migrated BMDMΦ on the lower side of the membrane were stained with crystal violet (Alfa Aesar).

Astrocytes-endothelial cells co-culture assay. Primary astrocytes were isolated from C57BL/6 mice between 2 and 3 days old¹⁸. The cerebral cortices were dissected and dissociated prior to plating in tissue culture flasks. The mixed glial cultures were grown in DMEM/F12 supplemented with 10% FBS, and 1% penicillin/streptomycin until reaching confluence. The flasks were then shaken at 200 rpm for 5 hours at 37° C. to get rid of microglia. After 4 rounds of shaking, the cells remaining in the flask were cultured as an enriched astrocyte primary culture which was confirmed by GFAP staining. For astrocytes-ECs co-culture, we used a transwell system where astrocytes were seeded in the lower chamber and BMECs were seeded in the upper chamber. 2×10⁴ non-reactive astrocytes or LPS-induced reactive astrocytes (LPS, 1,000 ng/ml for 18 hr¹⁹ were cultured in the transwell lower chamber with astrocytes medium (50% DMEM, 50% Ham's F-12 and 10% FBS) when they reached 90% confluency. Meanwhile, 2×10³ BMECs were cultured in an insert with 5% FBS in DMEM medium for 24 hr. Then, the inserts with the BMEC growths were placed into the lower chambers containing the astrocytes. These two cell types were co-cultured with astrocytes medium (50% DMEM, 50% Ham's F-12 and 1% FBS) for 72 hr and the BMECs in the insert were stained with Hoechst to label nuclei. The number of BMEC nuclei was then quantified using Nikon Element software. Negative control was included in this assay where only medium, no astrocytes were cultured with BMECs. Myelin-treated BMECs in the upper chamber were used as positive control.

In vitro endothelial-to-mesenchymal transition (endoMT) assays. 5×10³ BMECs were seeded in 24-well plate for endoMT induction. As positive control, recombinant TGF-β1 protein (long/ml) was added to BMECs for 72 hr to induce in vitro endoMT. Cells were considered spindle-shaped when the diameter at their longest axis to be 1.5-fold greater than the average diameter of untreated cobblestone BMECs. Myelin debris (1 mg/ml) was added to BMECs for 1, 3, 5, 7, and 10 days and culture medium was changed every 3 days, followed by morphological observation of spindle-shape cells. α-SMA staining was used to confirm the mesenchymal transition. Percentage of spindle-shaped cells and percentage of a-SMA⁺/CD31⁺ were quantified. To examine the role of TGF-β in myelin-induced endoMT, pan TGF-β neutralizing antibody (20 μg/ml) was added to BMECs together with myelin debris for 10 days and re-supplemented every 3 days.

Spinal cord BMEC injection. BMECs were treated with myelin debris for 72 hr, followed by wash to remove non-ingested myelin debris. Myelin-laden BMECs were fluorescently labeled after incubation for 1 hr with 50 μM CFSE in DMEM without serum. The CFSE-labeled BMECs were washed once for 5 min with 1×PBS containing 100 mM glycine and twice with 1×PBS. The CF SE-labeled BMECs were trypinsized and resuspended in cold 1×PBS. The CFSE signals in naïve-ECs and myelin-ECs were confirmed after labeling. The fluorescent intensity of CFSE in myelin-ECs were much more rapidly diluted than naïve-ECs after subculture (data not shown), probably due to robust proliferation of myelin-ECs, as shown in FIGS. 5A, 5B, 5C, 5C-1, 5D, 5D-1, 5E, 5F, 5F-1, 5G, 5H, 5I, 5J, and 5K. BMECs at 1×10⁵ cells in 0.5 μl were injected in T10 spinal cords of normal mice using a 33 gauge needle attached to Hamilton syringe. PBS injection was included as blank control. Myelin debris alone injection was included as a control to illustrate whether or not myelin debris, without ECs uptake, could elicit any responses when compared to myelin-EC injection. ECs cultured with myelin debris in the presence of 70° C. heated serum (IgG-inactivated), which demonstrated myelin-EC contacting without internalization, were included to illustrate whether or not blocking EC myelin uptake via IgG inactivation could abrogate any in vivo spinal cord responses induced by myelin-ECs. The spinal cords were minimally exposed to avoid strong mechanical injury that may cause any unfavorable interference with cell injection. After 7 days of injection, mice were anaesthetized and perfused, followed by collection of spinal cords for regular histology and immunostaining. To quantify proangiogenic response after injection, we stained Ki-67 and CD31 and counted the number of Ki-67⁺/CD31⁺ cells. Fluorescent intensities of Iba-1⁺ and GFAP⁺ cells were quantified for pro-inflammatory response.

Statistical analysis. The statistical significance of the difference between control and experimental groups was determined by unpaired Student's t-test, unless otherwise indicated using Prism 7 (Graphpad, San Diego, Calif.). Differences were considered statistically significant when p<0.05. * denotes p<0.05, ** for p<0.01 and *** for p<0.001 as shown in figures. Data were shown as mean±s.e.m.

### References For Materials and Methods (Only)

1. Young, W. Spinal cord contusion models. Prog Brain Res 137, 231-255 (2002).
2. Wu, H. et al. Caveolin-1 Is Critical for Lymphocyte Trafficking into Central Nervous System during Experimental Autoimmune Encephalomyelitis. The Journal of neuroscience: the official journal of the Society for Neuroscience 36, 5193-5199 (2016).
3. Mi, S. et al. LINGO-1 antagonist promotes spinal cord remyelination and axonal integrity in MOG-induced experimental autoimmune encephalomyelitis. Nature medicine 13, 1228-1233 (2007).
4. Wang, X. et al. Macrophages in spinal cord injury: phenotypic and functional change from exposure to myelin debris. Glia 63, 635-651 (2015).
5. Sun, X. et al. Myelin activates FAK/Akt/NF-kappaB pathways and provokes CR3-dependent inflammatory response in murine system. PLoS One 5, e9380 (2010).
6. Zeng, M. & Zhou, J. N. Roles of autophagy and mTOR signaling in neuronal differentiation of mouse neuroblastoma cells. Cellular signalling 20, 659-665 (2008).
7. Gitik, M., Liraz-Zaltsman, S., Oldenborg, P. A., Reichert, F. & Rotshenker, S. Myelin down-regulates myelin phagocytosis by microglia and macrophages through interactions between CD47 on myelin and SIRPalpha (signal regulatory protein-alpha) on phagocytes. J Neuroinflammation 8, 24 (2011).
8. Guo, L. et al. Rescuing macrophage normal function in spinal cord injury with embryonic stem cell conditioned media. Mol Brain 9, 48 (2016).
9. Hackett, A. R. et al. STAT3 and SOCS3 regulate NG2 cell proliferation and differentiation after contusive spinal cord injury. Neurobiology of disease 89, 10-22 (2016).
10. Bolger, A. M., Lohse, M. & Usadel, B. Trimmomatic: a flexible trimmer for Illumina sequence data. Bioinformatics 30, 2114-2120 (2014).
11. Kim, D. et al. TopHat2: accurate alignment of transcriptomes in the presence of insertions, deletions and gene fusions. Genome biology 14, R36 (2013).
12. Li, H. et al. The Sequence Alignment/Map format and SAMtools. Bioinformatics 25, 2078-2079 (2009).
13. Anders, S., Pyl, P. T. & Huber, W. HTSeq—a Python framework to work with high-throughput sequencing data. Bioinformatics 31, 166-169 (2015).
14. Love, M. I., Huber, W. & Anders, S. Moderated estimation of fold change and dispersion for RNA-seq data with DESeq2. Genome biology 15, 550 (2014).
15. Wu, H., Wang, C. & Wu, Z. A new shrinkage estimator for dispersion improves differential expression detection in RNA-seq data. Biostatistics 14 (2013).
16. Benjamini, Y. & Hochberg, Y. Controlling the false discovery rate: a practical and powerful approach to multiple testing. J R Stat Soc Ser B Methodol 57 (1995).
17. Wang, X. et al. MIF produced by bone marrow-derived macrophages contributes to teratoma progression after embryonic stem cell transplantation. Cancer Res 72, 2867-2878 (2012).
18. Wu, J., Wrathall, J. R. & Schachner, M. Phosphatidylinositol 3-kinase/protein kinase Cdelta activation induces close homolog of adhesion molecule L1 (CHL1) expression in cultured astrocytes. Glia 58, 315-328 (2010).
19. Chung, I. Y. & Benveniste, E. N. Tumor necrosis factor-alpha production by astrocytes. Induction by lipopolysaccharide, IFN-gamma, and IL-1 beta. Journal of immunology 144, 2999-3007 (1990).

All patents, patent applications, provisional applications, and publications referred to or cited herein are incorporated by reference in their entirety, including all figures and tables, to the extent they are not inconsistent with the explicit teachings of this specification.

Following are examples that illustrate procedures for practicing the invention. These examples should not be construed as limiting. All percentages are by weight and all solvent mixture proportions are by volume unless otherwise noted.

### Example 1—Microvessels in the Demyelinating Spinal Cords Contain Myelin Debris

Microvessels in the lesion epicenter are lost during the first two days after SCI, whereas ECs proliferate and give rise to newly formed microvessels from 3 days after injury, restoring microvessel density to a normal level by 1 week after SCI^{15,16}. We first examined whether these newly formed microvessels could engulf myelin debris. Myelin is intact in normal spinal cords and the uninjured spinal microvessels contain very little detectable myelin basic protein (MBP) (FIGS. 1A, 1A-1). By contrast, myelin debris, fragmented from myelin sheath following SCI, started to closely associate with newly formed microvessels in the lesion core as early as 3 to 5 days post SCI (FIG. 9), and became more apparent at 1 week after SCI (FIG. 1B). The x-z and y-z view of myelin debris distribution relative to microvessels revealed that myelin debris was indeed engulfed by microvessels (FIG. 1B-1, FIG. 9). Myelin debris-containing microvessels were frequently observed in the injured region and were much less frequently seen in the uninjured region of spinal cords following SCI (FIG. 1D, FIG. 9). Furthermore, neutral lipids, the myelin degradation products that are stained with Oil Red O (ORO), can be detected in microvessels at the injured core at 14 days after SCI (FIGS. 1E, 1E-1).

To investigate whether our observation extends to other demyelinating disorders, we established myelin oligodendrocyte glycoprotein (MOG)-induced EAE model in mice (FIG. 10A), a pre-clinical animal model of MS. The sagittal sections of EAE spinal cords demonstrated that the typical demyelinating lesions were concentrated in lumbar and thoracic cords (FIG. 10B). We observed the myelin debris-containing microvessels in T10-T12 demyelinating segments of spinal cords after 1 week of EAE induction (FIGS. 1C, 1C-1, 1D). These in vivo data from SCI and EAE models indicate that microvessels engulf myelin debris after demyelination.

### Example 2—In Vitro BMECs-Induced Microvessel-Like Structures Engulf Myelin Debris

Primary mouse brain microvascular endothelial cells (BMECs) grown on Matrigel as their substrate could form microvessel-like structures that mimics diverse aspects of microvessels²⁰. After 72 hr incubation with microvessel-like tubules, CFSE (Carboxyfluorescein succinimidyl ester)-labeled myelin debris was seen as scattered puncta around or within the tubules (FIG. 1F). A closer inspection of the distribution of myelin debris from the confocal x-z view of a capillary lumen revealed the apparent dynamics of myelin debris entry (FIG. 1F-1). Some myelin fragments appeared to be close to but were not in direct contact with the capillary surface, as indicated by a lack of co-localization with endothelial marker CD31 (FIG. 1F-1). Other myelin fragments were in the process of entering, whereas still others had completely transited the luminal membrane, showing partial or full co-localization with CD31 (FIG. 1F-1). These data form a picture of a plausible sequence of events and confirm that myelin debris could be internalized by microvessels.

### Example 3—In Vitro Engulfment of Myelin Debris by BMECs

We next investigated the kinetics and mechanisms of microvascular engulfment of myelin debris by using primary BMECs and a BMEC cell line bEnd.3. Both primary BMECs and bEnd.3 cells engulfed myelin debris in a time-dependent manner with predominant perinuclear distribution (FIGS. 2A, 2D). To quantitatively determine the efficiency and kinetics of myelin debris engulfment by BMECs, we used fluorescence-activated cell sorting (FACS) that counts the number of myelin-laden ECs (myelin-ECs), as well as a MBP ELISA assay to detect the intracellular MBP level. FACS results confirmed that primary BMECs were able to engulf myelin debris (FIG. 2B). MBP ELISA assay detected a significant amount of MBP in BMECs treated with myelin debris for 72 hr (FIG. 2C). The kinetics of myelin engulfment by bEnd.3 cells exhibited inefficient engulfment from 24-48 hr, efficient engulfment from 48-72 hr, and saturated engulfment from 72-96 hr (FIGS. 2E, 2F). As ECs are not professional phagocytes, it is not surprising that ECs engulfed myelin debris much slowly than BMDMΦ, the major cell type responsible for myelin debris clearance after SCI¹⁰, which showed rapid myelin engulfment as early as 1-3 hr (FIG. 11).

### Example 4—IgG Opsonization is Required for Effective Myelin Debris Engulfment by BMECs

Complement-3 receptor (CR3) and Mac-2 (Glactin-3) have been proposed as receptors for myelin debris phagocytosis by macrophages²¹ (FIG. 12A). CR3 is highly expressed on the macrophage surface, but was not detected on BMECs (data not shown). Blockage of CR3 or Mac-2 alone by neutralizing antibodies, or a combined blockage of CR3 and Mac2 did not affect myelin debris engulfment by BMECs (FIG. 2G, FIG. 12B). Low-density lipoprotein receptor-related protein 1 (LRP1), a proposed receptor for MBP²², was shown to mediate myelin debris phagocytosis by microglia, astrocytes and oligodendrocytes^{14,22}. However, we did not detect an obvious impairment of myelin debris engulfment by BMECs when using MBP-deficient myelin debris isolated from MBP-deficient mice (FIG. 13). These data suggest that CR3, Mac-2 or LRP1 has little or no role in mediating myelin debris engulfment in microvascular ECs.

Macrophage phagocytic capacity can be mediated by serum-derived opsonins including antibodies and complement proteins²³. To evaluate the role of opsonins on myelin debris engulfment, we cultured BMECs with myelin debris in different concentrations of serum. BMEC engulfment of myelin debris was stronger in the presence of 5% serum than that in 1% serum (FIG. 14), and was significantly reduced or even abolished after withdrawal of serum from interaction medium for phagocytosis (FIG. 14), suggesting the presence of factor(s) in serum that are required for BMEC engulfment of myelin debris. Inactivation of complements in serum by 56° C. heat-shock failed to prevent myelin uptake by BMECs, while serum IgG inactivation by 70° C. heat-shock^{24,25} significantly abrogated myelin debris engulfment as detected by immunostaining and MBP ELISA assay (FIGS. 2H, 2I), indicating serum IgG is required for myelin engulfment by BMECs. Supplement of IgG alone in serum-free medium or in IgG-inactivated serum rescued BMEC engulfment of myelin debris (FIG. 2I). Importantly, pre-coating/opsonization of myelin debris with IgG was sufficient for myelin engulfment by BMECs in the serum-free culture or IgG-inactivated serum culture (FIGS. 2H, 2I). These data indicate that opsonization of myelin debris by IgG is both necessary and sufficient for myelin engulfment by microvascular ECs.

### Example 5—Transcriptional Profiles of ECs after Engulfing Myelin Debris

To understand the cellular and molecular alterations in ECs after myelin debris uptake, we performed RNA sequencing of ECs with (myelin-ECs) or without myelin debris engulfment (naïve-ECs). Over 2,500 genes were significantly upregulated and over 4,000 genes were downregulated in myelin-ECs compared to naïve-ECs (FIG. 3A). The differentially expressed genes in myelin-ECs were enriched in a variety of processes or signaling pathways, mainly including metabolism, extracellular matrix formation, vesicle transport, inflammation, cell junctions, and Notch signaling among others (Table 1, FIGS. 3B, 3C).

Noteworthy, among the top 50 upregulated genes in myelin-ECs, collagen genes including Col1α2, Col5 α2, Col16α1, Col6α2, all of which were remarkably upregulated (FIG. 3B). Upregulation of these genes was additionally confirmed by q-PCR (FIG. 3D). The groups of inflammatory genes including interleukin (IL)-related genes (Il1rl1, Il4, Il5, Il13ra1) and chemokine related genes [Ccl2 (also known as Mcp-1), Cxcl1] were also upregulated in myelin-ECs (FIG. 3B). q-PCR analysis confirmed the increased gene expression of Il-4, Il-6,Mcp-1 and iNOS in myelin-ECs (FIG. 3E). Myelin debris uptake also upregulated vesicles-encoding genes for lysosomes, autophagosomes and endosomes (FIG. 3B). The downregulated genes in myelin-ECs are involved in Notch signaling pathway and cell adhesion/junction (FIG. 3C), which is related to endothelial angiogenesis and permeability, respectively.

TABLE 1
Top 25 KEGG pathways that were up-regulated and down-regulated
in myelin-ECs.

| up-regulated | | down-regulated | |
|---|---|---|---|
| Pathway Name | Adj P | Pathway Name | AdjP |
| Metabolic pathways | 4.69e−08 | Pathways in cancer | 9.51e−23 |
| Protein digestion and | 9.22e−06 | Focal adhesion | 1.97e−21 |
| absorption | | | |
| Focal adhesion | 1.96e−05 | Regutation of actin | 2.08e−19 |
| | | cytoskeleton | |
| ECM-receptor | 0.0001 | Endocytosis | 2.12e−17 |
| interaction | | | |
| Cytokine-cytokine | 0.0014 | MAPK signaling | 3.84e−13 |
| receptor interaction | | pathway | |
| Arachidonic acid | 0.0014 | Axon guidance | 1.66e−12 |
| metabolism | | | |
| Propanoate metabolism | 0.0014 | ECM-receptor interaction | 2.38e−12 |
| p53 signaling pathway | 0.0024 | Adherens junction | 2.38e−12 |
| Phagosome | 0.0054 | Notch signaling pathway | 1.72e−09 |
| TGF-beta signaling | 0.0058 | GnRH signaling pathway | 2.58e−09 |
| pathway | | | |
| Allograft rejection | 0.0062 | ErbB signaling pathway | 2.77e−09 |
| Tight junction | 0.0062 | Tight junction | 8.26e−09 |
| Hedgehog signaling | 0.0062 | Arrhythmogenic right | 1.98e−08 |
| pathway | | ventricular | |
| | | cardiomyopathy (ARVC) | |
| Glutathione | 0.0062 | Calcium signaling | 1.98e−08 |
| metabolism | | pathway | |
| MAPK signaling | 0.0084 | Phophatidylinositol | 3.81e−08 |
| pathway | | signaling system | |
| Regulation of actin | 0.0103 | Wnt signaling pathway | 4.56e−08 |
| cytoskeleton | | | |
| Autoimmune thyroid | 0.0108 | Bacterial invasion of | 9.35e−08 |
| disease | | epithelial cells | |
| Ribosome | 0.0128 | Small cell lung cancer | 1.31e−07 |
| Melanoma | 0.0128 | Amoebiasis | 1.31e−07 |
| Complement and | 0.0146 | Dilated cardiomyopathy | 1.62e−07 |
| coagulation cascades | | | |
| Salivary secretion | 0.0148 | Protein processing in | 1.62e−07 |
| | | endoplasmic reticulum | |
| Axon guidance | 0.0160 | Fe gamma R-mediated | 1.77e−07 |
| | | phagocytosis | |
| Alzheimer's disease | 0.0160 | Insulin signaling pathway | 1.85e−07 |
| Pathways in cancer | 0.0174 | Hypertrophic | 4.61e−07 |
| | | cardiomyopathy (HCM) | |
| Pyruvate metabolism | 0.0178 | ABC transporters | 4.61e−07 |

### Example 6—Engulfed Myelin Debris is Delivered to Lysosomal Degradation System Through Autophagy Pathway

The perinuclear localization of engulfed myelin debris within BMECs (FIGS. 2A, 2D) prompted us to examine its subcellular localization. We first examined whether myelin debris co-localized with lysosomes, the perinuclear organelles that break down cellular materials, because gene expression in lysosomal subunits was upregulated in myelin-ECs (FIG. 3B). Myelin debris was predominantly delivered to lysosomes as revealed by co-localization between the majority of myelin particles and puncta positive for the lysosomal marker Lysotracker red (FIGS. 4A, 4A-1).

Myelin debris uptake increased the size of the lysosomes, especially those containing myelin debris (FIGS. 4A, 4A-1, 4B). Lysosomes in naïve-ECs were 0.60±0.02 μm in diameter (FIGS. 4A, 4A-1, 4B). However, lysosomes that contained myelin debris (arrow) were 1.35±0.03 μm, while lysosomes containing no myelin debris (arrowhead) in myelin-ECs were maintained around 0.64±0.01 μm in diameter (FIGS. 4A, 4A-1, 4B). These data indicate that myelin debris is preferentially targeted to lysosomes, where myelin debris is likely to be degraded.

We then asked through which route(s) myelin debris is delivered to lysosomes. Cargo can be delivered through endocytosis or autophagy pathway to lysosomes for degradation²⁶. We first examined whether myelin debris colocalizes with endosomes in BMECs. There was almost no or little co-localization between myelin debris and the early (Rab5)/late (Rab7) endosomes (FIGS. 4C, 4D), suggesting that endosomal machinery is not the primary route for transport of myelin debris to lysosomes.

RNA sequencing data revealed upregulation of autophagy genes including Gabarapl2, Gabarap, Atg12, LC3b, Atg5 and Atg3 in myelin-ECs (FIG. 3B). We next set out to determine the possible role of the autophagy pathway in delivering myelin debris to lysosomes. Myelin-ECs induced a significant increase of autophagosome formation (LC3b-puncta), while BMECs fed with sufficient nutrient showed very few LC3b-puncta (negative control), and nutrient withdrawal by starvation induced robust formation of LC3b-puncta (positive control) (FIG. 4E). A closer look at the relative localization between myelin debris and LC3-puncta revealed that majority of myelin debris was in contact with autophagosomes (FIGS. 4C, 4D). Myelin debris also induced and showed significant co-localization with puncta positive for GABARAP (FIGS. 4C, 4D), another autophagy marker that is conjugated to autophagosomal membrane²⁷. The western blot analysis also showed that myelin debris uptake induced LC3-I conversion to LC3-II (FIG. 4F), an indicator of autophagosomes formation²⁸. Moreover, myelin debris uptake caused autophagy substrate p62 degradation (FIG. 4F), indicating autophagy induction by myelin debris.

We next examined whether genetically or pharmacologically inhibiting autophagy-lysosome pathway could block myelin degradation into neutral lipids. We generated EC cell lines deficient for autophagy by knocking out the Atg5 gene, a core autophagy gene, using CRISPR-Cas9 technique (FIGS. 15A, 15B, 4G). The Atg5 knockout BMECs were viable with no apparent cell death (FIG. 15D) or no cell growth defects (FIG. 5I), and thus could be maintained as stable cell line. The Atg5 knockout BMECs failed to generate LC3⁺ puncta (FIG. 4H) and induced LC3 conversion (FIG. 4I), as well as accumulated p62 and ubiquitin (FIGS. 4I, 15C), verifying the knockout is functional. Atg5 knockout BMECs failed to degrade the engulfed myelin debris into neutral lipids (FIGS. 4J, 4K). Consistently, either blocking autophagy using 3-MA, an inhibitor of autophagosome formation, or inhibition of lysosomal activity with chloroquine, an inhibitor of lysosomal acidification, significantly inhibited myelin degradation into neutral lipids in myelin-ECs without causing apparent cell toxicity (FIGS. 4J, 4K, 15D). These genetic or pharmacological data indicate that autophagy-lysosome pathway is required for engulfed myelin debris degradation. We then tested whether additional supply of autophagosomes to myelin-ECs could accelerate myelin degradation by rapamycin or nutrient starvation treatment, which are known to stimulate autophagosomes formation. Indeed, rapamycin or starvation accelerated myelin degradation in myelin-ECs (FIGS. 4J, 4K). All of these data indicate that in ECs myelin debris induces autophagosome formation, which is required for the delivery of myelin debris to lysosomes for degradation.

### Example 7—Microvessels are Enlarged in SCI and EAE Model

Although it is known that microvessel density is remarkably increased during the subacute phase of SCI^{15,16}, little attention has been paid to the change in the morphology and structure of the newly formed microvessels. Compared to the microvessels with a mean diameter of 8.17±0.41 μm in normal spinal cords, the microvessels in the injured core increased the mean diameter to 16.66±0.51 μm after 1 week post SCI (FIGS. 5A, 5B, 16A). These dilated microvessels were reproducibly seen in the injured core following 1, 4, 6, 8 and 10 weeks following SCI (FIG. 5B). The dilated microvessels were not only seen in the core lesions, but also seen in the marginal regions (FIGS. 5A, 5B). The dilated microvessels in the injury region were proliferative, as indicated by Ki-67, a marker for cell proliferation (FIGS. 5C, 5C-1). Interestingly, microvessels in demyelinated regions were also enlarged (FIGS. 5D, 5D-1) and proliferative in the spinal cords from EAE mice (FIG. 5E). These data indicate that enlarged microvessels are common in demyelinating diseases.

### Example 8—Engulfment and Autophagic Processing of Myelin Debris by ECs Promotes ECs Proliferation and Angiogenesis

The spatial difference in the microvascular size suggests the presence of lesion-related factors that could stimulate microvascular growth. We showed that myelin debris uptake significantly increased the Ki-67⁺ proliferative cells in both primary BMECs and the bEnd.3 cell line (FIGS. 5F, 5F-1). Cell populations were consistently increased after myelin debris uptake in a time-dependent manner (FIG. 5G). The highly proliferative capacity of myelin-ECs was further confirmed by an in vivo matrigel angiogenesis assay. Subcutaneous injection of matrigel plugs containing myelin-ECs stimulated extensive angiogenesis as observed microscopically and as measured by CD31 staining (FIG. 5H). Similarly, in in vivo spinal cord angiogenesis assay, myelin-ECs injected into normal spinal cord remained highly proliferative and appeared integrated within microvasculature (FIGS. 16B, 16B-1). It is noteworthy that either injection of myelin alone or injection of ECs (ECs were exposed to, but not able to engulf myelin debris in the presence of IgG-inactivated serum) failed to induce angiogenesis (FIGS. 16B, 16B-1). Moreover, in vitro myelin-induced EC proliferation was abrogated after Atg5 knockout (FIG. 5I), indicating that autophagic degradation of myelin debris is required for EC proliferation. These data indicate that myelin debris gains proangiogenic potential only after being engulfed and intracellularly processed in ECs. The proangiogenic potential of myelin-ECs could likely be attributed to the increased expression of vascular endothelial growth factor (VEGF), a potent proangiogenic factor (FIG. 5J) as VEGF neutralization blocked myelin-induced EC proliferation (FIG. 5K).

### Example 9—ECs Engulfment of Necrotic Neuronal Cell Bodies Inhibits Cell Proliferation

In addition to the myelin debris present in lesion core, dead cells, like necrotic neuronal cell corpses are also present following acute SCI. We wondered whether the dead cells could be another lesion-localized factor in microvascular EC proliferation and angiogenesis. We firstly found that BMECs could not only engulf small particles like myelin debris, but also take up the large necrotic neuronal cell bodies, which were generated by 60° C. heating of neuro-2a (N2A) cells-differentiated neuronal cells (FIGS. 17A, 17B). BMEC engulfment of necrotic neuronal bodies was less efficient than BMDMΦ (FIG. 17C). Interestingly, BMEC nucleus was squeezed and distorted by the engulfed large necrotic cell body (FIG. 17B). This cell-in-cell interaction significantly inhibited BMEC growth (FIG. 17D). The opposite effects of myelin debris and necrotic neuronal cell bodies on EC proliferation suggest the specificity of myelin uptake in regulating EC functions. We tested this specificity in two additional assays. First, we tested a pathogen, fungal zymosan, for EC uptake and proliferation. Surprisingly, zymosan, which could be efficiently engulfed by BMDMΦ (FIG. 17G), could not be engulfed by BMECs (FIG. 17H) and had no effect on EC proliferation (data not shown). Second, we tested reactive astrocytes, which surround SCI lesion and form glia scar as a potential indirect factor for EC proliferation. It is unknown whether the reactive astrocytes have any effects on microvessel growth in lesion core. We attempted to mimic the in vivo indirect interaction in a transwell co-culture assay where BMECs and astrocytes were cultured in distinct spaces (FIGS. 18A, 18B). We found that either resting or reactive astrocytes induced by LPS had no significant effects on BMEC proliferation (FIG. 18C). Altogether, these data indicate that myelin debris, which mainly distribute within the injury epicenter following SCI, is a lesion factor that promotes the microvascular EC proliferation and could thus contribute to the microvessel dilation after SCI.

### Example 10—Myelin Debris Uptake by Microvascular ECs Stimulates Inflammatory Responses

Since neovascularization and inflammation occur simultaneously and persist in the injury site, we sought to examine whether myelin debris-primed ECs promote leukocyte infiltration. Using mice reconstituted with GFP⁺ bone marrow cells, we can track the distribution of the GFP⁺ bone marrow-derived cells (BMDCs), which have been previously identified as BMDMΦ mainly in the injured spinal cords¹⁰. Consistent with previous findings, normal spinal cords had little or no GFP⁺ BMDMΦ infiltration. However, BMDMΦ infiltrated in the injured core and closely associated with newly formed microvessels following 3 days post SCI (FIGS. 6A, 19A). The number of microvessel-associated BMDMΦ increased from 3 days to 1 or 2 weeks post SCI (FIGS. 6A, 19A), correlating with formation of new microvessels, especially enlarged microvessels in the injury core. Similarly in 1-week EAE mice spinal cord, we observed close association between infiltrated Iba-1⁺ macrophages and enlarged microvessels in demyelinating regions (FIG. 6B). This correlation suggests macrophage recruitment is dependent on the newly formed microvessels, which may serve as a portal to facilitate BMDMΦ entry. Supporting this, a closer look at the infiltrated BMDMΦ in zoomed confocal images and 3-D reconstructed images showed that some BMDMΦ were bordering the outer surface of microvessels or were just entering the spinal cord (FIG. 6A-1), indicating that they were in the process of or had completed transmigration across microvascular endothelium towards spinal cord parenchyma.

We next determined whether myelin debris uptake activates ECs, which is a critical step for the adhesion and trans-endothelial migration of leukocytes into tissues. There were a greater number of BMDMΦ adhering to the myelin-ECs monolayer than that to the naïve-ECs monolayer (FIGS. 6C, 6C-1), probably due to the increased expression of adhesion molecules, like vascular cell adhesion molecule-1 (VCAM-1) in myelin-ECs (FIG. 6D). ECs did not induce BMDMΦ adhesion when ECs were exposed to myelin debris without IgG opsonization of myelin debris (FIGS. 19C, 6C-1), which allowed ECs to be exposed to myelin debris, but not have the ability to engulf it. We further showed that ECs with myelin uptake but no intracellular degradation (Atg5 knockout EC uptake myelin debris) did not increase BMDMΦ adhesion either (FIGS. 19C, 6C-1), indicating that only intracellularly processed myelin debris is able to activate ECs and induce BMDMΦ adhesion, while simple myelin exposure or myelin uptake alone is not sufficient. Besides macrophage adhesion, conditioned media from myelin-ECs remarkably stimulated BMDMΦ chemotaxis, while conditioned media from naïve-ECs showed little or no effect on BMDMΦ recruitment (FIG. 6E). This suggests the presence of endothelial-derived chemokines from myelin-ECs stimulating BMDMΦ migration. We found in both primary BMECs and BMEC cell line that myelin debris significantly increased endothelial secretion of MCP-1 (FIGS. 6F, 6F-1), the major chemokine for macrophage recruitment²⁹. In addition to MCP-1, myelin-ECs upregulated other pro-inflammatory medicators, including interleukin-related factors and other chemokines (FIGS. 3B, 3E), which may together contribute to BMDMΦ infiltration. Furthermore, myelin-ECs appeared to activate BMDMΦ, as evidenced by upregulated IL-6 expression in BMDM after being exposed to myelin-EC conditioned media (FIG. 6G). We then examined the inflammatory potential of myelin-ECs by injection of naïve-ECs or myelin-ECs into normal spinal cords. Myelin-ECs injection increased the number of Iba-1⁺ macrophages/microglia compared to naïve-ECs injection (FIGS. 6H, 19E). Compared to naïve-EC injection, myelin-ECs injected into the normal spinal cords also activated astrocytes, as revealed by increased GFAP intensity (FIGS. 6H, 19F) and hypertrophy of astrocytic processes (FIG. 6H). It is noteworthy that either injection of myelin alone or injection of ECs (ECs were exposed to but not able to engulf myelin debris in the presence of IgG-inactivated serum) failed to induce inflammation in vivo (FIGS. 19D, 19E, 19F), confirming that myelin debris uptake by ECs, but not simple exposure, promotes pro-inflammatory responses. Furthermore, we tested whether ECs engulfment of necrotic neuronal cell bodies, another possible lesion-related factor has any effects on inflammation, but found no significant change in MCP-1 gene expression (FIG. 17E), indicating that EC engulfment of myelin debris specifically promotes inflammation.

### Example 11—Enlarged Microvessels and ECs Contribute to Fibrotic Components Production

After SCI, a fibrotic scar forms and occupies the injury core, which is typically characterized by extensive deposition of collagen³⁰ and fibronectin³¹. However, little is known about the cellular origin of such fibrotic scar in the injury core. Given the highly upregulated collagen genes in myelin-ECs (FIGS. 3B, 3D), we therefore examined the spatiotemporal relationship between microvascular ECs and collagen/fibronectin in the injured spinal cords. A low level of collagen 1 (Col1) expression in the normal cord was detected with a close association with microvessels (FIG. 7A). However, at 6 weeks after SCI, a dense Col1 matrix was present at the injury core and its expression pattern intimately resembled that of enlarged microvessels (FIG. 7B). There was almost no expression of fibronectin throughout the normal spinal cords (FIG. 7D). Some, but not all microvessels are closely associated with fibronectin expression at 6 weeks after SCI (FIG. 7E), which was different from the robust and close distribution between most microvessels and collagen. Similarly, a dense Col1 or fibronectin matrix was seen co-localizing well with enlarged microvessels in the spinal cords from 15-day EAE mice (FIGS. 7C, 7F). Our in vivo data from two demyelinating models support that microvascular ECs could be a novel source of fibrotic scar formation through endothelial production of collagen and fibronectin.

We next investigated the mechanisms by which microvascular ECs in the injured core become pro-fibrotic. RNA sequencing identified the top upregulated genes implicated in fibrosis, including collagen genes Col1α2, Col5 α2, Col16α1, and Col6α2 in myelin-ECs (FIG. 3B), suggesting that myelin debris has the potential to stimulate microvascular ECs to produce fibrotic components in the injury core. BMECs treated with myelin debris for 1 day did not significantly increase Col1 expression (data not shown) or fibronectin expression (FIG. 7I), however, prolonged treatment with myelin debris significantly increased expression of Col1 (FIGS. 7G, 7G-1) and fibronectin (FIG. 7I) to a level similar to that of transforming growth factor β1 (TGF-β1) treatment, a strong inducer of collagen or fibronectin production³². Myelin debris also increased Col1 expression in microvessel-like structures when BMECs were seeded on matrigel (FIG. 7H), supporting the notion that myelin debris stimulates microvascular production of pro-fibrotic proteins in lesion core.

### Example 12—Engulfment and Autophagic Processing of Myelin Debris by ECs Induces Endothelial-to-Mesenchymal Transition (endoMT)

Interestingly, treatment with myelin debris for 10 days reduced CD31 expression in some BMECs (FIG. 7G), a phenotype resembling endothelial-to-mesenchymal transition (endoMT). EndoMT is characterized by downregulation of CD31 and acquisition of mesenchymal cell phenotype (e.g. expression of α-SMA [alpha-smooth muscle actin], secretion of collagen and fibronectin) and has been strongly implicated in tissue fibrosis in various diseases³³. BMECs at the basal condition exhibited the characteristic polygonal cobblestone-like morphology (FIGS. 8A, 8C), while after treatment with TGF-β1, a strong inducer of endoMT³³, BMECs elongated and exhibited the same spindle-like morphology as fibroblasts (FIGS. 8A, 8C), a morphological change indicating the induction of endoMT. Similar to TGF-β, BMECs exposure to myelin debris for 10 days showed spindle-like morphology (FIGS. 8A, 8C). Additionally, exposure to TGF-β1 or myelin debris markedly downregulated CD31 expression and strongly induced a-SMA expression in BMECs by q-PCR, immunostaining and western blot assays (FIGS. 8B, 8D, 8E, 8F). While EC engulfment of myelin debris induced α-SMA expression and thus endoMT, EC engulfment of necrotic neuronal cell bodies did not induce α-SMA expression (FIG. 17F), indicating the specificity of myelin uptake by ECs in endoMT induction. We further found that engulfment of myelin debris upregulated TGF-β1 expression by q-PCR analysis (FIG. 8G), and blockade of TGF signaling by a pan-TGF-β neutralizing antibody abrogated myelin-induced phenotypes associated with endoMT, including morphological change (FIGS. 8A, 8C) and a-SMA expression (FIGS. 8B, 8D). This indicated that myelin debris induced endoMT via TGF signaling. Autophagic processing of myelin debris was crucial for myelin-induced endoMT because Atg5 knockout BMECs failed to show morphological change (FIGS. 8A, 8C) and α-SMA expression (FIGS. 8B, 8D). Noteworthy, at 6 weeks after SCI and 15 days of EAE, microvessels showed co-localization between CD31 and a-SMA, indicative of in vivo endoMT following demyelination (FIG. 8H). Despite the lack of more direct in vivo endoMT evidence from endothelial lineage tracking system, our in vitro data showed that myelin debris generated after SCI stimulates endothelial-derived fibrotic components, probably via endoMT.

While professional phagocytes such as BMDMΦ and microglia are the major players in the clearance of myelin debris generated after demyelination, our in vitro and in vivo data demonstrated that microvascular ECs can act as amateur phagocytes to engulf myelin debris. We revealed that myelin debris uptake and autophagic processing by microvascular ECs have more important functions. EC uptake and processing of myelin debris cause a series of sequential events associated with disease progression, including inflammation, angiogenesis, and fibrotic scar formation.

Most of our knowledge of myelin debris phagocytosis comes from studies on macrophages and microglia. Receptors such as CR3, Mac-2, and LRP-1 are involved in myelin debris phagocytosis by macrophages/microglia²¹. Our study showed that ECs do not employ these receptors for myelin debris uptake. The “naked” myelin debris is not recognized by ECs and only IgG-opsonized myelin debris can be engulfed effectively, suggesting IgG receptors (FcγRs) are involved in myelin debris engulfment by ECs. The family of FcγRs is highly expressed in macrophages to regulate a multitude of immune responses by interaction with IgG, immune complexes, and opsonized particles or cells³⁴. It is likely that ECs only express a small amount of FcγRs that engage in the engulfment of IgG-opsonized myelin debris, and this may account for the limited phagocytic capacity of ECs compared to strong phagocytosis of myelin debris by BMDMΦ. Compromised BBB leads to leakage of IgG in the injured area³⁵, which may be the source of IgG for oposinization. Given the fact that brain ECs and other antigen presenting cells are able to engulf myelin debris and present myelin antigens to lymphocytes³⁶, it is speculated that the specific antibodies may further opsonize myelin debris and facilitate its engulfment. Endogenous antibodies have been shown to promote the rapid clearance of myelin debris in mouse³⁷, but it is unknown which cell type(s) benefit from the opsonization by antibody for myelin debris uptake. Because IgG opsonization is required for myelin debris uptake by ECs, but not BMDMΦ (data not shown), we propose that ECs, rather than BMDMΦ, are the major cell type that relies on antibody opsonization of myelin debris for in vivo myelin debris clearance.

Autophagy is a fundamental degradative pathway for degradation of intracellular proteins and organelles. One feature of autophagy is the formation of autophagosomes, which engulf cargoes and upon fusion with lysosomes form autolysosomes leading to the degradation of the enclosed materials²⁸. Autophagy has recently emerged as an alternative mechanism for myelin debris clearance in Schwann cells^{38,39}. Using autophagy-deficient ECs, we show that autophagy is required for myelin debris degradation in ECs. Furthermore, autophagic processing of myelin debris is crucial for proangiogenic, pro-inflammatory, and pro-fibrotic responses. However, if either ECs contact myelin debris but do not internalize it, or internalize myelin debris without autophagic processing, it does not elicit those responses, indicating that myelin debris causes consequences only after being engulfed and intracellularly processed. It would be valuable to investigate the in vivo role of EC autophagy in demyelinating disorders using endothelium-specific atg5 or atg7 knockout mouse model⁴⁰.

The major form of vascular change in the injury area is angiogenesis during chronic stages of SCI. The newly formed microvessels are structurally abnormal, appearing dilated and more disorganized (FIGS. 5A, 5B, 5C, 5C-1, 5D, 5D-1, 5E, 5F, 5F-1, 5G, 5H, 5I, 5J, 5K, and 16A). The dilated and abnormal microvessels are also seen in mouse EAE model. The mechanisms and biological outcomes for these vascular abnormalities are poorly understood after demyelination. We demonstrated that myelin debris is one critical lesion-related factor that causes excessive EC proliferation, which may contribute to microvessel dilation at injury sites. Interestingly, the dilated microvessels in the injured spinal cords recapitulated the microvessels in mice lacking pericytes in early stage of SCI⁴¹, a cellular constituent in the neurovascular unit that has been recently reported to constrict microvessels⁴². Therefore, an alternative explanation for the microvessel dilation could be that these newly formed microvessels have defects in pericytes maturation or/and coverage, which thus fail to constrict microvessels and lead to microvessel dilation.

One of the most important features of neuroinflammation is the leukocyte recruitment from the blood circulation into the CNS, which requires the activation of ECs through an increased expression of adhesion molecules and secretion of cytokines/chemokines in ECs⁴³. Our study demonstrates that myelin debris engulfment activates microvascular ECs by increasing expression of adhesion molecules such as VCAM-1 and a variety of cytokines/chemokines that could facilitate BMDMΦ recruitment to injury site. Our RNA sequencing data suggests myelin debris might also promote BMDMΦ influx to injury site by increasing microvascular permeability to leukocytes, as indicated by the downregulation of genes related to cell junctions in myelin-ECs.

The fibrotic scars in the central region of injury sites, characterized by the excessive accumulation of pro-fibrotic proteins such as collagen and fibronectin, have been known to inhibit axon regeneration⁴⁴. Fibroblasts, which are prominent in the injured epicenter, contribute to fibrotic scar formation by stimulating the production of collagen³⁰ and fibronectin³¹. However, little is known about the cellular origin of fibroblasts in contusive injured spinal cords, whose dura is generally left intact and do not permit the invasion of meningeal fibroblasts into lesions^{30,31,45}. Our study demonstrated that enlarged microvessels contribute to the significant deposition of fibrotic components in SCI and EAE models. Soderblom et al reported contribution of perivascular fibroblasts from larger-diameter microvessels (in our study, we referred to them as enlarged or dilated microvessels) to Col1α1 production and fibrotic scar formation using Col1α1-GFP transgene, which is consistent with our results³⁰. The exact cellular identity of perivascular fibroblasts is not very clear, given that different cell types can share the same cell maker and some cell types can have further sub-types. The activated fibroblasts, or myofibroblasts, may arise from other sources including resident fibroblasts, perivascular pericytes, bone marrow-derived precursors and others⁴⁶.

It has been recently reported that ECs have greater plasticity than was previously acknowledged, and can acquire fibroblast-like properties by undergoing endoMT³³. Our study demonstrated that microvascular ECs could become fibroblasts-like cells after myelin uptake via endoMT. This suggests microvascular ECs are an additional source of fibroblasts or fibroblasts-like cells for fibrotic scar formation at the SCI lesion core. It is likely that the Col1α1-GFP transgene labels only a subset of collagen-producing cells, which may account for the 27% of CD13⁺ Col1α1-GFP cells³⁰. CD13 is a marker that labels ECs and other cell types like pericytes. Interestingly, it takes a few days for myelin debris to significantly increase expression of fibronectin, collagen, and α-SMA in microvascular ECs, coinciding with the delayed accumulation of perivascular fibroblasts at the injury core⁴⁷. EndoMT occurs during SCI and EAE, as indicated by the expression of mesenchymal marker α-SMA in microvessels. Endothelial lineage-tracking system could be applied in the future to confirm the endothelial origin of these fibroblasts-like cells in microvessels. We further determined that myelin debris induces endoMT via TGF-β1-dependent mechanism. We showed myelin debris upregulates TGF-β1 expression and TGF-β signaling is required for myelin debris-induced endoMT. TGF signaling has been known as a master regulator of endoMT³³ and participates in the formation of fibrotic scars in the injury site^{48,49}. The TGF-β signaling is activated in several cell types within SCI lesion, including macrophages, astrocytes as well as ECs in blood vessels^{44,50}. Thus, we propose that TGF signaling-mediated endoMT in ECs may underlie the effects of TGF signaling on fibrotic scar formation in SCI lesions.

We have shown that microvessels and lining microvascular ECs act as amateur phagocytes to engulf myelin debris generated by CNS disorders associated with prominent demyelination. Mechanistically, we determined that IgG opsonization of myelin debris is required for efficient uptake by microvascular ECs. The engulfed myelin debris is then delivered through autophagy-lysosome pathway for intracellular degradation. Functionally, engulfment and autophagy-dependent processing of myelin debris by microvascular ECs contribute to three critical processes that are closely associated with CNS demyelinating disorders: robust angiogenesis that results in excessive and abnormal microvessels, chronic inflammation, and endothelial-mediated fibrosis that most likely takes place through endoMT (FIGS. 20A, 20B). Therefore, it may be possible to reverse the effects of myelin-ECs by targeting these particular processes (e.g., myelin debris uptake, autophagy and endoMT).

### Exemplified Embodiments

Exemplified embodiments of the invention include, but are not limited to:

### Embodiment 1

A method for treating a demyelinating condition in a human or animal subject, comprising administering an agent to the subject that inhibits vascular endothelial cell phagocytosis.

### Embodiment 2

The method of embodiment 1, wherein the demyelinating condition is associated with a neural injury.

### Embodiment 3

The method of embodiment 2, wherein the neural injury is an injury of the peripheral nervous system (PNS), central nervous system (CNS), or both.

### Embodiment 4

The method of embodiment 3, wherein the neural injury is an injury of the CNS.

### Embodiment 5

The method of embodiment 4, wherein neural injury is a spinal cord injury (SCI).

### Embodiment 6

The method of embodiment 1, wherein the subject has the demyelinating condition at the time of administering the agent to the subject, and the agent is administered as therapy.

### Embodiment 7

The method of embodiment 1, wherein the subject does not have the demyelinating condition at the time of administering the agent to the subject, and the agent is administered as prophylaxis to prevent, delay onset or recurrence, or to reduce the severity of a potential demyelinating condition.

### Embodiment 8

The method of any preceding embodiment, wherein the demyelinating condition is selected from among spinal cord injury, traumatic brain injury, multiple sclerosis (MS), Alzheimer's disease, autoimmune encephalomyelitis, acute disseminated encephalomyelitis (ADEM), Balo's disease (concentric sclerosis), Charcot-Marie-Tooth disease (CMT), Guillaian Barre Syndrome (GBS), HTLV-1-associated myelopathy (HAM), neuromyelitis optica (Devic's disease), Schilder's disease, transverse myelitis, congenital metabolic disorder with demyelination, neuropathy with abnormal myelination, drug-induced demyelination, radiation-induced demyelination, hereditary demyelination condition, prion-induced demyelination, encephalitis-induced demyelination, and chronic inflammatory demyelinating neuropathy.

### Embodiment 9

The method of embodiment 8, wherein the chronic inflammatory demyelinating neuropathy is selected from among chronic Immune Demyelinating Polyneuropathy (CIDP); multifocal CIDP; multifocal motor neuropathy (MMN); anti-MAG Syndrome (Neuropathy with IgM binding to Myelin-Associated Glycoprotein); GALOP Syndrome (Gait disorder Autoantibody Late-age Onset Polyneuropathy); anti-sulfatide antibody syndrome; anti-GM2 gangliosides antibody syndrome; POEMS syndrome (Polyneuropathy Organomegaly Endocrinopathy or Edema M-protein Skin changes); perineuritis; and IgM anti-GD1b ganglioside antibody syndrome.

### Embodiment 10

The method of any preceding embodiment, wherein the agent inhibits the autophagy-lysosome pathway in vascular endothelial cells.

### Embodiment 11

The method of any preceding embodiment, wherein the agent is a MAP kinase inhibitor (e.g., SP600125, U0126, SB202190, and SB203580), PI3K inhibitor (e.g., 3-methyladenine, LY294002, and Wortmannin), protein biosynthesis inhibitor (e.g., cycloheximide), Vacuolar-type H (+)-ATPase (V-ATPase) inhibitor (e.g., bafilomycin), lysosomal lumen alkalyzer (e.g., chloroquine, hydroxychloroquine, NH4C1, neutral red, Lys01,and Lys05), acid protease inhibitor (e.g., leupeptin, E64d, and pepstatin A), or endosome inhibitor (e.g., Bafilomycin A1, and chloroquine).

### Embodiment 12

The method of embodiment 10, wherein the agent that inhibits the autophagy-lysosome pathway is selected from among: 3-methyladenine (3-MA), CPD18 (a.k.a. 3-methyl-6-(3-methylpiperidin-1-yl)-3H-purine), bafilomycin A1, chloroquine, hydroxychloroquine, LY294002 (a.k.a. 2-(4-Morpholinyl)-8-phenyl-4H-1-benzopyran-4-one), SB202190, SB203580, SC79, Wortmannin (a.k.a. SL-2052), SP600125 (a.k.a. 1,9-Pyrazoloanthrone), U0126 (a.k.a. (2Z,3Z)-2,3-bis[amino-(2-aminophenyl)sulfanylmethylidene]butanedinitrile), MHY1485 (a.k.a. 4,6-dimorpholino-N-(4-nitrophenyl)-1,3,5-triazin-2-amine), autophinib, azithromycin, (±)-Bay K 8644, concanamycin A (a.k.a. folimycin), DBeQ (a.k.a. N2,N4-Bis(phenylmethyl)-2,4-quinazolinediamine), E 64d (a.k.a. (2 S,3 S)-3-[[[(1 S)-3-Methyl-1-[[(3-methylbutyl)amino]carbonyl]butyl]amino]carbonyl]-2-oxiranecarboxylic acid ethyl ester), edaravone (a.k.a. MCI 186), GW 4064 (a.k.a. 3-[2-[2-Chloro-4-[[3-(2,6-dichlorophenyl)-5-(1-methylethyl)-4-isoxazolyl]methoxy]phenyl]ethenyl]benzoic acid), Mdivi 1 (a.k.a. 3-(2,4-Dichloro-5-methoxyphenyl)-2,3-dihydro-2-thioxo-4(1H)-quinazolinone), ML 240 (2-(2-Amino-1H-benzimidazole-1-yl)-8-methoxy-N-(phenylmethyl)-4-quinazolinamine), MRT 67307 (a.k.a. N-[3-[[5-Cyclopropyl-2-[[3-(4-morpholinylmethyl)phenyl]amino]-4-pyrimidinyl]amino]propyl]cyclobutanecarboxamide), MRT 68601 (N-[3-[[5-Cyclopropyl-2-[[4-(4-morpholinyl)phenyl]amino]-4-pyrimidinyl]amino]propyl]cyclobutanecarboxamide), MRT 68921 (a.k.a. N-[3-[[5-Cyclopropyl-2-[(1,2,3,4-tetrahydro-2-methyl-6-isoquinolinyl)amino]-4-pyrimidinyl]amino]propyl]cyclobutanecarboxamide), NMS 873 (3-[3-(Cyclopentylthio)-5-[[[2-methyl-4′-(methylsulfonyl)[1,1′-biphenyl]-4-yl]oxy]methyl]-4H-1,2,4-triazol-4-yl]pyridine), nocodazole (a.k.a. [5-(2-Thienylcarbonyl)-1H-benzimidazol-2-yl]carbonic acid, methyl ester), pepstatin A, apautin 1 (a.k.a. 6-Fluoro-N-[(4-Fluorophenyl)methyl]-4-quinazolinamine), taxol (a.k.a. paclitaxel), vinblastine (a.k.a. vincaleukoblastine), xanthohumol (a.k.a. (2E)-1-[2,4-Dihydroxy-6-methoxy-3-(3-methyl-2-buten-1-yl)phenyl]-3-(4-hydroxyphenyl)-2-propen-1-one), Tetrahydroacridine 33 (a.k.a. 6-Chloro-N-(1-ethylpiperidin-4-yl)-1,2,3,4-tetrahydroacridin-9-amine), Thapsigargin (a.k.a. 3 S,3aR,4S,6S,6aR,7S,8S,9b S)-6-(Acetyloxy)-4-(butyryloxy)-3,3a-dihydroxy-3,6,9-trimethyl-8-{[(2Z)-2-methylbut-2-enoyl]oxy}-2-oxo-2,3,3a,4,5,6,6a,7,8,9b-decahydroazuleno[4,5-b]furan-7-yl octanoate), ARN5187 (a.k.a. 4-(((1-(2-Fluorophenyl)cyclopentyl)-amino)methyl)-2-((4-methylpiperazin-1-yl)methyl)phenol), Spautin-1 (6-fluoro-N-[4-fluorobenzyl]quinazolin-4-amine), N-acetyl cysteine (a.k.a. NAC), L-asparagine (a.k.a. (S)-2-Aminosuccinic acid 4-amide), Catalase from human erythrocytes (a.k.a. H₂O₂:H₂O₂ oxidoreductase), E-64d (a.k.a. (2S,3S)-trans-Epoxysuccinyl-L-leucylamido-3-methylbutane ethyl ester, GMX1778 (a.k.a. N-[6-(4-Chlorophenoxy)hexyl]-N′-cyano-N″-4-pyridinyl-guanidine), Leupeptin (a.k.a. Acetyl-Leu-Leu-Arg-al), and SBI-0206965 (a.k.a. 2-((5-Bromo-2-((3,4,5-trimethoxyphenyl)amino)pyrimidin-4-yl)oxy)-N-methylbenzamide).

### Embodiment 13

The method of embodiment 10, wherein the agent that inhibits the autophagy-lysosome pathway is selected from among an antisense, RNA-interference molecule (e.g., shRNA), and microRNA(e.g., MiR-101 and MiR-30a) that targets a component of the autophagy-lysosome pathway (e.g., ATG5) in vascular endothelial cells by blocking or reducing the component's expression, as a genetic intervention.

### Embodiment 14

The method of embodiment 10, wherein the agent that inhibits the autophagy-lysosome pathway is an agent that inhibits ATG5 in vascular endothelial cells.

### Embodiment 15

The method of any preceding embodiment, wherein the agent depletes immunoglobulin G (IgG) or inactivates IgG in the subject.

### Embodiment 16

The method of embodiment 15, wherein the agent that inactivates IgG is an enzyme that hydrolyzes IgG, such as EndoS or IdeS.

### Embodiment 17

The method of any one of embodiments 1 to 9, wherein the agent inhibits immunoglobulin G (IgG) opsonization of myelin debris.

### Embodiment 18

The method of embodiment 17, wherein the agent blocks the Fc receptor on vascular endothelial cells in the subject.

### Embodiment 19

The method of embodiment 18, wherein the Fc-gamma receptor is the FcRn receptor.

### Embodiment 20

The method of embodiment 18 or 19, wherein the agent is a monoclonal or polyclonal antibody, antigen-binding fragment thereof, peptide, or small molecule that binds to the Fc-gamma receptor.

### Embodiment 21

The method of embodiment 15, wherein the agent that depletes IgG is a B-cell-attenuating agent, such as bortezomib or rituximab.

### Embodiment 22

The method of any preceding embodiment, wherein the agent is administered locally at a anatomical site.

### Embodiment 23

The method of embodiment 22, wherein the desired anatomical site is a site where demyelination exists or is at risk of occurring.

### Embodiment 24

The method of any preceding embodiment, wherein the agent is administered directly into the cerebrospinal fluid (CSF) of the subject.

### Embodiment 25

A packaged dosage formulation for treating a demyelinating condition, comprising an agent that inhibits vascular endothelial cell phagocytosis in a pharmaceutically acceptable dosage.

### Embodiment 26

A kit for treating a demyelinating condition, comprising, in one or more containers, at least one agent that inhibits vascular endothelial cell phagocytosis.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### REFERENCES (OTHER THAN MATERIALS & METHODS SECTIONS)

1. Ahuja, C. S., et al. Traumatic spinal cord injury. Nature reviews. Disease primers 3, 17018 (2017).
2. Schwab, M. E. & Bartholdi, D. Degeneration and regeneration of axons in the lesioned spinal cord. Physiological reviews 76, 319-370 (1996).
3. Franklin, R. J. & Ffrench-Constant, C. Remyelination in the CNS: from biology to therapy. Nature reviews. Neuroscience 9, 839-855 (2008).
4. Chen, M. S., et al. Nogo-A is a myelin-associated neurite outgrowth inhibitor and an antigen for monoclonal antibody IN-1. Nature 403, 434-439 (2000).
5. Filbin, M. T. Myelin-associated inhibitors of axonal regeneration in the adult mammalian CNS. Nature reviews. Neuroscience 4, 703-713 (2003).
6. Kotter, M. R., Li, W. W., Zhao, C. & Franklin, R. J. Myelin impairs CNS remyelination by inhibiting oligodendrocyte precursor cell differentiation. The Journal of neuroscience: the official journal of the Society for Neuroscience 26, 328-332 (2006).
7. Syed, Y. A., et al. Antibody-mediated neutralization of myelin-associated EphrinB3 accelerates CNS remyelination. Acta Neuropathol 131, 281-298 (2016).
8. Jeon, S. B., Yoon, H. J., Park, S. H., Kim, I. H. & Park, E. J. Sulfatide, a major lipid component of myelin sheath, activates inflammatory responses as an endogenous stimulator in brain-resident immune cells. Journal of immunology 181, 8077-8087 (2008).
9. Sun, X., et al. Myelin activates FAK/Akt/NF-kappaB pathways and provokes CR3-dependent inflammatory response in murine system. PLoS One 5, e9380 (2010).
10. Wang, X., et al. Macrophages in spinal cord injury: phenotypic and functional change from exposure to myelin debris. Glia 63, 635-651 (2015).
11. Napoli, I. & Neumann, H. Protective effects of microglia in multiple sclerosis. Experimental neurology 225, 24-28 (2010).
12. Neumann, H., Kotter, M. R. & Franklin, R. J. Debris clearance by microglia: an essential link between degeneration and regeneration. Brain: a journal of neurology 132, 288-295 (2009).
13. Guo, L., et al. Rescuing macrophage normal function in spinal cord injury with embryonic stem cell conditioned media. Mol Brain 9, 48 (2016).
14. Ponath, G., et al. Myelin phagocytosis by astrocytes after myelin damage promotes lesion pathology. Brain: a journal of neurology 140, 399-413 (2017).
15. Ng, M. T., Stammers, A. T. & Kwon, B. K. Vascular disruption and the role of angiogenic proteins after spinal cord injury. Transl Stroke Res 2, 474-491 (2011).
16. Whetstone, W. D., Hsu, J. Y., Eisenberg, M., Werb, Z. & Noble-Haeusslein, L. J. Blood-spinal cord barrier after spinal cord injury: relation to revascularization and wound healing. Journal of neuroscience research 74, 227-239 (2003).
17. Rengarajan, M., Hayer, A. & Theriot, J. A. Endothelial Cells Use a Formin-Dependent Phagocytosis-Like Process to Internalize the Bacterium Listeria monocytogenes. PLoS pathogens 12, e1005603 (2016).
18. Dini, L., et al. Phagocytosis of apoptotic bodies by liver endothelial cells. J Cell Sci 108 (Pt 3), 967-973 (1995).
19. Steffan, A. M., Gendrault, J. L., McCuskey, R. S., McCuskey, P. A. & Kim, A. Phagocytosis, an unrecognized property of murine endothelial liver cells. Hepatology 6, 830-836 (1986).
20. Arnaoutova, I. & Kleinman, H. K. In vitro angiogenesis: endothelial cell tube formation on gelled basement membrane extract. Nat Protoc 5, 628-635 (2010).
21. Smith, M. E. Phagocytic properties of microglia in vitro: implications for a role in multiple sclerosis and EAE. Microscopy research and technique 54, 81-94 (2001).
22. Gaultier, A., et al. Low-density lipoprotein receptor-related protein 1 is an essential receptor for myelin phagocytosis. J Cell Sci 122, 1155-1162 (2009).
23. Hart, S. P., Smith, J. R. & Dransfield, I. Phagocytosis of opsonized apoptotic cells: roles for ‘old-fashioned’ receptors for antibody and complement. Clinical and experimental immunology 135, 181-185 (2004).
24. Jones, F. S. The Effect of Heat on Antibodies. The Journal of experimental medicine 46, 291-301 (1927).
25. Vermeer, A. W. & Norde, W. The thermal stability of immunoglobulin: unfolding and aggregation of a multi-domain protein. Biophysical journal 78, 394-404 (2000).
26. Tooze, S. A., Abada, A. & Elazar, Z. Endocytosis and autophagy: exploitation or cooperation? Cold Spring Harb Perspect Biol 6, a018358 (2014).
27. Shpilka, T., Weidberg, H., Pietrokovski, S. & Elazar, Z. Atg8: an autophagy-related ubiquitin-like protein family. Genome Biol 12, 226 (2011).
28. Mizushima, N., Yoshimori, T. & Levine, B. Methods in mammalian autophagy research. Cell 140, 313-326 (2010).
29. Deshmane, S. L., Kremlev, S., Amini, S. & Sawaya, B. E. Monocyte chemoattractant protein-1 (MCP-1): an overview. Journal of interferon & cytokine research: the official journal of the International Society for Interferon and Cytokine Research 29, 313-326 (2009).
30. Soderblom, C., et al. Perivascular fibroblasts form the fibrotic scar after contusive spinal cord injury. The Journal of neuroscience: the official journal of the Society for Neuroscience 33, 13882-13887 (2013).
31. Zhu, Y., Soderblom, C., Trojanowsky, M., Lee, D. H. & Lee, J. K. Fibronectin Matrix Assembly after Spinal Cord Injury. J Neurotrauma 32, 1158-1167 (2015).
32. Ignotz, R. A. & Massague, J. Transforming growth factor-beta stimulates the expression of fibronectin and collagen and their incorporation into the extracellular matrix. The Journal of biological chemistry 261, 4337-4345 (1986).
33. Piera-Velazquez, S., Mendoza, F. A. & Jimenez, S. A. Endothelial to Mesenchymal Transition (EndoMT) in the Pathogenesis of Human Fibrotic Diseases. Journal of clinical medicine 5 (2016).
34. Guilliams, M., Bruhns, P., Saeys, Y., Hammad, H. & Lambrecht, B. N. The function of Fcgamma receptors in dendritic cells and macrophages. Nat Rev Immunol 14, 94-108 (2014).
35. Winkler, E. A., Bell, R. D. & Zlokovic, B. V. Central nervous system pericytes in health and disease. Nature neuroscience 14, 1398-1405 (2011).
36. Lopes Pinheiro, M. A., et al. Internalization and presentation of myelin antigens by the brain endothelium guides antigen-specific T cell migration. eLife 5 (2016).
37. Vargas, M. E., Watanabe, J., Singh, S. J., Robinson, W. H. & Barres, B. A.
Endogenous antibodies promote rapid myelin clearance and effective axon regeneration after nerve injury. Proceedings of the National Academy of Sciences of the United States of America 107, 11993-11998 (2010).
38. Brosius Lutz, A., et al. Schwann cells use TAM receptor-mediated phagocytosis in addition to autophagy to clear myelin in a mouse model of nerve injury. Proceedings of the National Academy of Sciences of the United States of America 114, E8072-E8080 (2017).
39. Gomez-Sanchez, J. A., et al. Schwann cell autophagy, myelinophagy, initiates myelin clearance from injured nerves. The Journal of cell biology 210, 153-168 (2015).
40. Torisu, T., et al. Autophagy regulates endothelial cell processing, maturation and secretion of von Willebrand factor. Nature medicine 19, 1281-1287 (2013).
41. Hellstrom, M., et al. Lack of pericytes leads to endothelial hyperplasia and abnormal vascular morphogenesis. The Journal of cell biology 153, 543-553 (2001).
42. Li, Y., et al. Pericytes impair capillary blood flow and motor function after chronic spinal cord injury. Nature medicine 23, 733-741 (2017).
43. Wu, F., Liu, L. & Zhou, H. Endothelial cell activation in central nervous system inflammation. Journal of leukocyte biology 101, 1119-1132 (2017).
44. Kawano, H., et al. Role of the lesion scar in the response to damage and repair of the central nervous system. Cell and tissue research 349, 169-180 (2012).
45. Fernandez, E. & Pallini, R. Connective tissue scarring in experimental spinal cord lesions: significance of dural continuity and role of epidural tissues. Acta neurochirurgica 76, 145-148 (1985).
46. Hinz, B., et al. The myofibroblast: one function, multiple origins. The American journal of pathology 170, 1807-1816 (2007).
47. Zhu, Y., et al. Hematogenous macrophage depletion reduces the fibrotic scar and increases axonal growth after spinal cord injury. Neurobiology of disease 74, 114-125 (2015).
48. Logan, A., et al. Effects of transforming growth factor beta 1 on scar production in the injured central nervous system of the rat. The European journal of neuroscience 6, 355-363 (1994).
49. Yoshioka, N., et al. Small molecule inhibitor of type I transforming growth factor-beta receptor kinase ameliorates the inhibitory milieu in injured brain and promotes regeneration of nigrostriatal dopaminergic axons. Journal of neuroscience research 89, 381-393 (2011).
50. Fee, D. B., et al. Traumatic brain injury increases TGF beta RII expression on endothelial cells. Brain research 1012, 52-59 (2004).

What is claimed is:

1. A method for treating a demyelinating condition in a human or animal subject, comprising administering an agent to the subject that inhibits vascular endothelial cell phagocytosis.

2. The method of claim 1, wherein the demyelinating condition is associated with a neural injury.

3. The method of claim 2, wherein the neural injury is an injury of the peripheral nervous system (PNS), central nervous system (CNS), or both.

4. The method of claim 3, wherein neural injury is a spinal cord injury (SCI).

5. The method of claim 1, wherein the subject has the demyelinating condition at the time of administering the agent to the subject, and the agent is administered as therapy.

6. The method of claim 1, wherein the subject does not have the demyelinating condition at the time of administering the agent to the subject, and the agent is administered as prophylaxis to prevent, delay onset or recurrence, or to reduce the severity of a potential demyelinating condition.

7. The method of claim 1, wherein the demyelinating condition is selected from among spinal cord injury, traumatic brain injury, multiple sclerosis (MS), Alzheimer's disease, autoimmune encephalomyelitis, acute disseminated encephalomyelitis (ADEM), Balo's disease (concentric sclerosis), Charcot-Marie-Tooth disease (CMT), Guillaian Barre Syndrome (GB S), HTLV-1-associated myelopathy (HAM), neuromyelitis optica (Devic's disease), Schilder's disease, transverse myelitis, congenital metabolic disorder with demyelination, neuropathy with abnormal myelination, drug-induced demyelination, radiation-induced demyelination, hereditary demyelination condition, prion-induced demyelination, encephalitis-induced demyelination, and chronic inflammatory demyelinating neuropathy.

8. The method of claim 7, wherein the chronic inflammatory demyelinating neuropathy is selected from among chronic Immune Demyelinating Polyneuropathy (CIDP); multifocal CIDP; multifocal motor neuropathy (MMN); anti-MAG Syndrome (Neuropathy with IgM binding to Myelin-Associated Glycoprotein); GALOP Syndrome (Gait disorder Autoantibody Late-age Onset Polyneuropathy); anti-sulfatide antibody syndrome; anti-GM2 gangliosides antibody syndrome; POEMS syndrome (Polyneuropathy Organomegaly Endocrinopathy or Edema M-protein Skin changes); perineuritis; and IgM anti-GD1b ganglioside antibody syndrome.

9. The method of claim 1, wherein the agent is a MAP kinase inhibitor, PI3K inhibitor, protein biosynthesis inhibitor, Vacuolar-type H (+)-ATPase (V-ATPase) inhibitor, lysosomal lumen alkalyzer, acid protease inhibitor, or endosome inhibitor.

10. The method of claim 1, wherein the agent inhibits the autophagy-lysosome pathway in vascular endothelial cells and is selected from among: 3-methyladenine (3-MA), CPD18 (also known as 3-methyl-6-(3-methylpiperidin-1-yl)-3H-purine), bafilomycin A1, chloroquine, hydroxychloroquine, LY294002 (also known as 2-(4-Morpholinyl)-8-phenyl-4H-1-benzopyran-4-one), SB202190, SB203580, SC79, Wortmannin (also known as SL-2052), SP600125 (also known as 1,9-Pyrazoloanthrone), U0126 (also known as (2Z,3Z)-2,3-bis[amino-(2-aminophenyl)sulfanylmethylidene]butanedinitrile), MHY1485 (also known as 4,6-dimorpholino-N-(4-nitrophenyl)-1,3,5-triazin-2-amine), autophinib, azithromycin, (±)-Bay K 8644, concanamycin A (also known as folimycin), DBeQ (also known as N2,N4-Bis(phenylmethyl)-2,4-quinazolinediamine), E 64d (also known as (2S,3S)-3-[[[(1S)-3-Methyl-1-[[(3-methylbutyl)amino]carbonyl]butyl]amino]carbonyl]-2-oxiranecarboxylic acid ethyl ester), edaravone (also known as MCI 186), GW 4064 (also known as 3-[2-[2-Chloro-4-[[3-(2,6-dichlorophenyl)-5-(1-methylethyl)-4-isoxazolyl]methoxy]phenyl]ethenyl]benzoic acid), Mdivi 1 (also known as 3-(2,4-Dichloro-5-methoxyphenyl)-2,3-dihydro-2-thioxo-4(1H)-quinazolinone), ML 240 (2-(2-Amino-1H-benzimidazole-1-yl)-8-methoxy-N-(phenylmethyl)-4-quinazolinamine), MRT 67307 (also known as N-[3-[[5-Cyclopropyl-2-[[3-(4-morpholinylmethyl)phenyl]amino]-4-pyrimidinyl]amino]propyl]cyclobutanecarboxamide), MRT 68601 (N-[3-[[5-Cyclopropyl-2-[[4-(4-morpholinyl)phenyl]amino]-4-pyrimidinyl]amino]propyl]cyclobutanecarboxamide), MRT 68921 (also known as N-[3-[[5-Cyclopropyl-2-[(1,2,3,4-tetrahydro-2-methyl-6-isoquinolinyl)amino]-4-pyrimidinyl]amino]propyl]cyclobutanecarboxamide), NMS 873 (3-[3-(Cyclopentylthio)-5-[[[2-methyl-4′-(methylsulfonyl)[1,1′-biphenyl]-4-yl]oxy]methyl]-4H-1,2,4-triazol-4-yl]pyridine), nocodazole (also known as [5-(2-Thienylcarbonyl)-1H-benzimidazol-2-yl]carbonic acid, methyl ester), pepstatin A, apautin 1 (also known as 6-Fluoro-N-[(4-Fluorophenyl)methyl]-4-quinazolinamine), taxol (also known as paclitaxel), vinblastine (also known as vincaleukoblastine), xanthohumol (also known as (2E)-1-[2,4-Dihydroxy-6-methoxy-3-(3-methyl-2-buten-1-yl)phenyl]-3-(4-hydroxyphenyl)-2-propen-1-one), Tetrahydroacridine 33 (also known as 6-Chloro-N-(1-ethylpiperidin-4-yl)-1,2,3,4-tetrahydroacridin-9-amine), Thapsigargin (also known as 3S,3aR,4S,6S,6aR,7S,8S,9b S)-6-(Acetyloxy)-4-(butyryloxy)-3,3a-dihydroxy-3,6,9-trimethyl-8-{[(2Z)-2-methylbut-2-enoyl]oxy}-2-oxo-2,3,3a,4,5,6,6a,7,8,9b-decahydroazuleno[4,5-b]furan-7-yl octanoate), ARN5187 (also known as 4-(((1-(2-Fluorophenyl)cyclopentyl)-amino)methyl)-2-((4-methylpiperazin-1-yl)methyl)phenol), Spautin-1 (6-fluoro-N-[4-fluorobenzyl]quinazolin-4-amine), N-acetyl cysteine (also known as NAC), L-asparagine (also known as (S)-2-Aminosuccinic acid 4-amide), Catalase from human erythrocytes (also known as H₂O₂:H₂O₂ oxidoreductase), E-64d (also known as (2S,3S)-trans-Epoxysuccinyl-L-leucylamido-3-methylbutane ethyl ester, GMX1778 (also known as N-[6-(4-Chlorophenoxy)hexyl]-N′-cyano-N″-4-pyridinyl-guanidine), Leupeptin (also known as Acetyl-Leu-Leu-Arg-al), and SBI-0206965 (also known as 2-((5-Bromo-2-((3,4,5-trimethoxyphenyl)amino)pyrimidin-4-yl)oxy)-N-methylbenzamide).

11. The method of claim 1, wherein the agent inhibits the autophagy-lysosome pathway in vascular endothelial cells and is selected from among an antisense, RNA-interference molecule, and microRNA that targets a component of the autophagy-lysosome pathway in vascular endothelial cells by blocking or reducing the component's expression, as a genetic intervention.

12. The method of claim 1, wherein the agent inhibits the autophagy-lysosome pathway in vascular endothelial cells and inhibits ATG5 in vascular endothelial cells.

13. The method of claim 1, wherein the agent depletes immunoglobulin G (IgG) or inactivates IgG in the subject.

14. The method of claim 13, wherein the agent that inactivates IgG is an enzyme that hydrolyzes IgG, such as EndoS or IdeS.

15. The method of claim 1, wherein the agent inhibits immunoglobulin G (IgG) opsonization of myelin debris.

16. The method of claim 15, wherein the agent blocks the Fc receptor on vascular endothelial cells in the subject.

17. The method of claim 13, wherein the agent depletes IgG and is a B-cell-attenuating agent.

18. The method of claim 1, wherein the agent is administered locally at a anatomical site, or where demyelination exists or is at risk of occurring, or is administered directly into the cerebrospinal fluid (CSF) of the subject.

19. A packaged dosage formulation for treating a demyelinating condition, comprising an agent that inhibits vascular endothelial cell phagocytosis in a pharmaceutically acceptable dosage.

20. A kit for treating a demyelinating condition, comprising, in one or more containers, at least one agent that inhibits vascular endothelial cell phagocytosis.
